(19) United States
(12) Patent Application Publication
(10) Pub. No.: US 2019/0046506 A1
(43) Pub. Date: Feb. 14, 2019

(54) USE OF 5HT2A INVERSE AGONISTS FOR TREATING PSYCHOSIS

(71) Applicant: Axovant Sciences GmbH, Basel (CH)

(72) Inventors: Lawrence Tim FRIEDHOFF, Rivervale, NJ (US); Brante SAMPEY, New York, NY (US)

(21) Appl. No.: 16/101,299

(22) Filed: Aug. 10, 2018

Related U.S. Application Data
(60) Provisional application No. 62/543,827, filed on Aug. 10, 2017.

(51) Int. Cl. A61K 31/415; A61K 9/00; A61P 25/18; A61P 25/16; A61P 25/28

(52) U.S. Cl. CPC A61K 31/415; A61K 9/0053; A61P 25/28; A61P 25/16; A61P 25/18

(57) ABSTRACT

Disclosed herein are methods of using a 5-HT_{2A} serotonin receptor inverse agonist for the prophylaxis and/or treatment of psychosis or psychotic symptoms in an individual comprising administering to said individual in need thereof a therapeutically effective amount of said 5-HT_{2A} serotonin receptor inverse agonist.

### CROSS REFERENCE TO RELATED APPLICATIONS

This application claims the priority benefit under 35 U.S.C. 119(e) of U.S. Provisional Application No. 62/543,827 filed Aug. 10, 2017, the disclosure of which is incorporated by reference in its entirety.

### SUMMARY

The present invention is directed to the use of a 5-HT_{2A} serotonin receptor inverse agonist for the prophylaxis and/or treatment of psychosis or psychotic symptoms in an individual comprising administering to said individual in need thereof a therapeutically effective amount of said 5-HT_{2A} serotonin receptor inverse agonist. In some embodiments, administering to said individual in need thereof a therapeutically effective amount of said 5-HT_{2A} serotonin receptor inverse agonist does not result in a worsening of psychosis or psychotic symptoms such as, but not limited to, hallucinations and delusions. In some embodiments, administering to said individual in need thereof a therapeutically effective amount of said 5-HT_{2A} serotonin receptor inverse agonist results in an improvement of psychosis or psychotic symptoms such as, but not limited to, hallucinations and delusions. In some embodiments, administering to said individual in need thereof a therapeutically effective amount of said 5-HT_{2A} serotonin receptor inverse agonist results in an improvement of psychosis or psychotic symptoms.

### BRIEF DESCRIPTION OF THE DRAWINGS

FIG. 1 is a graph showing the potency for antagonizing 5-HT-induced βarrestin2 recruitment.

FIG. 2 depicts analysis of the data obtained from the Versamax open field activity monitors in a mouse study based on total distance travelled.

FIG. 3 depicts analysis of the data obtained from the Versamax open field activity monitors in a mouse study based on Horizontal beam breaks.

FIG. 4 depicts analysis of the data obtained from the Versamax open field activity monitors in a mouse study based on Stereotypy counts (beam breaks in X, Y, Z).

FIG. 5 depicts data related to DOI-induced head twitch.

FIG. 6 depicts analysis of the data obtained from intracellular calcium release.

### DETAILED DESCRIPTION

Any methods and materials similar or equivalent to those described herein can be used in the practice or testing of embodiments of the present invention. The methods, devices, and materials described herein are exemplary in nature.

Many people suffer from psychosis or psychotic symptoms from a variety of causes. There is a significant unmet need for therapies to treat psychosis and psychotic symptoms, regardless of cause, but particularly when associated with diseases such as Parkinson's disease, dementia with Lewy bodies and Parkinson's disease dementia, and other conditions. These patients can often suffer from psychotic symptoms and/or neuropsychiatric symptoms including hallucinations and delusions. Most existing antipsychotics aim to treat these symptoms by blocking the dopamine D₂ receptor to limit dopamine activity. However, this activity against dopamine can exacerbate motor problems common to these patients. As a result, most existing antipsychotics are avoided altogether or used with extreme caution in patients with Parkinson's disease, dementia with Lewy bodies, and Parkinson's disease dementia. This application pertains to treating psychosis or psychotic symptoms, regardless of cause.

Inverse agonists/antagonists at the serotonin 2A receptor (5HT2AR) have proven to be highly effective, mainstay therapies for the treatment of psychosis for decades. Many atypical antipsychotics, including clozapine, have been explored for their efficacy in treating a wide range of neuropsychiatric deficits. However, atypical antipsychotics target multiple receptors, and in the case of clozapine, demonstrate activity at dopamine and 5HT2C receptors. Described herein are methods of using 5HT2AR inverse agonists described to treat psychosis or psychotic symptoms.

Psychosis is an abnormal condition of the mind that involves a “loss of contact with reality”. People experiencing psychosis may exhibit personality changes and thought disorder. Depending on its severity, this may be accompanied by unusual or bizarre behavior, as well as difficulty with social interaction and impairment in carrying out daily life activities. There are several subtypes of psychosis including, but not limited to cycloid psychosis, menstrual psychosis, post partum psychosis, monthematic delusions, myxedematous psychosis, stimulant psychosis, tardive psychosis, shared psychosis, and others. Psychosis may be the result of or symptomatic of an underlying psychiatric disorder, stress, use of psychoactive drugs, alcohol consumption, medication, trauma, a medical condition, or combination of these and/or other causes. Psychosis can even be seen in normal states, often, but not always, accompanying stress, such as hallucinating when falling asleep or waking (hypnagogic and hypnopompic hallucinations), during bereavement, as a result of sleep deprivation, or sensory deprivation or impairment, from caffeine intoxication, or from other events.

Examples of psychiatric disorders that can cause psychosis or psychotic symptoms include, but are not limited to, schizophrenia, affective (mood) disorders, including depression, severe depression, or bipolar disorder, schizoaffective disorder, brief psychotic disorder, chronic hallucinatory psychosis, schizotypal personality disorder, paranoid personality disorder, schizoid personality disorder, borderline personality disorder, major depressive disorder, bipolar disorder, especially in manic or mixed episodes, post-traumatic stress disorder, induced delusional disorder, obsessive-compulsive disorder, dissociative disorders, and others.

Psychotic symptoms include but are not limited to hallucinations, delusions, catatonia, thought disorder, impairment of social cognition, and combinations thereof. Hallucinations are often thought of as visual, but may occur with any of the senses. Delusions are false beliefs held without adequate evidence, and may be paranoid or otherwise in nature. Catatonia describes a profoundly agitated state often characterized by either complete immobility and interaction with the world or excessive motor behavior and mental focus so as to prevent experiencing reality.

Nelotanserin, a 5HT2A inverse agonist with 80-fold selectivity over 5HT2C and inactive in a panel of 66 receptors and ion channels, is being investigated for treating psychosis or psychotic symptoms, particularly in patients diagnosed with Lewy body dementia who experience frequent visual hallucinations, and is particularly well suited for use in the treatment methods described herein.

There is a significant unmet need for therapies to treat psychosis and psychotic symptoms, regardless of cause, but particularly when associated with diseases such as Parkinson's disease, dementia with Lewy bodies and Parkinson's disease dementia, and other conditions. These patients can often suffer from psychotic symptoms and/or neuropsychiatric symptoms including hallucinations and delusions. Most existing antipsychotics aim to treat these symptoms by blocking the dopamine D₂ receptor to limit dopamine activity. However, this activity against dopamine can exacerbate motor problems. As a result, most existing antipsychotics are avoided altogether or used with extreme caution in patients with Parkinson's disease, dementia with Lewy bodies, and Parkinson's disease dementia. Surprisingly and unexpectedly, Applicant has found that the 5-HT_{2A} serotonin receptor inverse agonist nelotanserin has been effective in treating psychotic symptoms in mouse models.

Nelotanserin was characterized as an antagonist of 5-HT2A stimulation in the DiscoveRx PathHunter βarrestin2 enzyme fragment complementation assay using h5HT2A-Dx-U2OS cells (n=6-7). Cells were incubated with nelotanserin or clozapine for 90 minutes at 37° C. followed by addition of DiscoverX detection reagent for 60 minutes, after which luminescence was measured.

In vivo antagonist effects were assessed in C57Bl/6J male mice by 1: inhibition of DOI (1-(2,5-dimethoxy 4-iodophenyl)-2-amino propane hydrochloride)-induced head twitch response and 2: inhibition of PCP-induced hyperlocomotor activation. In the DOI-induced head twitch assay, mice were administered nelotanserin (1-10 mg/kg, i.p.) or vehicle 30 minutes prior to administration of the 5HT2A receptor agonist, DOI (1 mg/kg, i.p.) and observed for head twitch response over 1 hour. PCP-induced hyperlocomotion was measured in open field activity monitor chambers. Mice were administered nelotanserin (10 mg/kg, i.p.), clozapine (1 mg/kg, i.p.), or vehicle (i.p.), and their activity was monitored for 30 minutes before administration of either PCP (10 mg/kg, i.p.) or saline (i.p.) followed by an additional 30 minutes of activity monitoring. All studies were performed in a blinded and randomized manner and potencies were calculated. Clozapine was used as the basis for comparison.

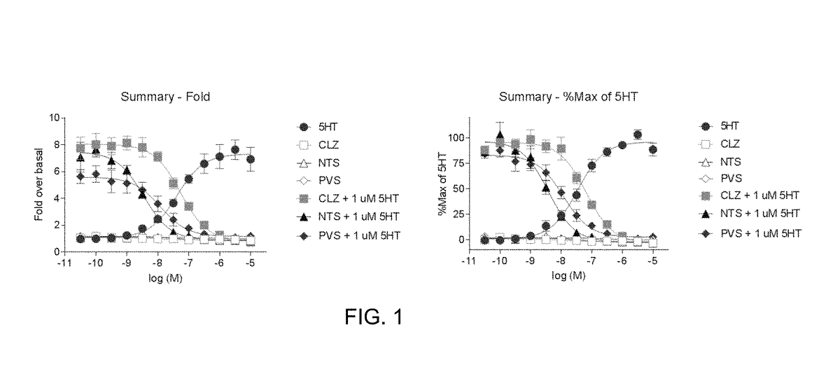

Nelotanserin inhibited serotonin-induced 5HT2AR-β-arrestin 2 interactions, acting as a potent antagonist of the recruitment of β-arrestin 2 to the 5HT2A receptor (IC50 (SEM): 3.74 nM (±0.65)), and was found to be more potent than clozapine (IC50 (SEM): 67.8 nM (±12.1)). In mice, nelotanserin blocked DOI-induced head twitches in a dose-dependent manner (ED50 (95% CL): 2.4 mg/kg (1.3-4.4)), demonstrating potent in vivo antagonism of the 5HT2A agonist-induced behavior. Finally, in a mouse model of psychosis-like behavior, PCP-induced hyperlocomotor activity was effectively inhibited by nelotanserin (ED50 (95% CL): 3.0 mg/kg (1.7-5.5)), without any effect of nelotanserin on locomotor behavior on its own. This effect was comparable to the results obtained with clozapine. No significant effects on head twitches or locomotor activity were observed upon treatment of nelotanserin followed by vehicle. FIGS. 1-6 summarize depict these results.

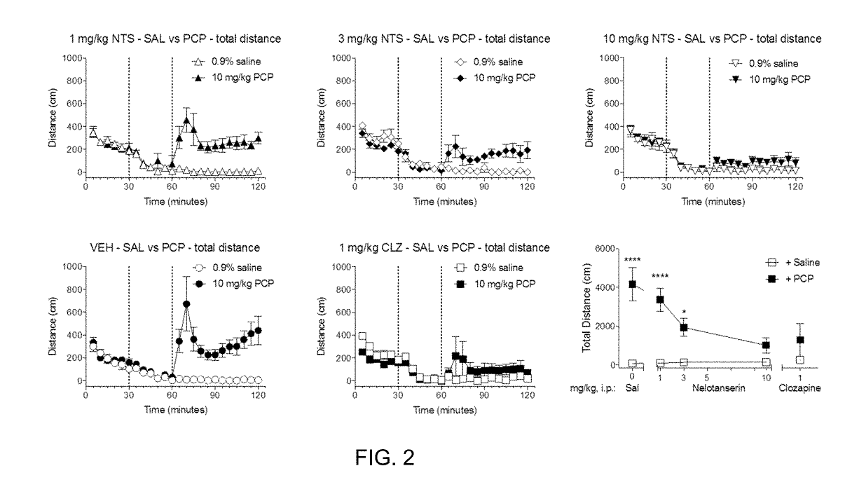

The data depicted in FIGS. 2-5, summarizes Mouse data—Male C57BL6J mice from JAX˜12 weeks old. Studies were randomized and investigator was blinded. The figures contain three ways of analyzing the data obtained from the Versamax open field activity monitors on the same cohort of mice. The infrared beams in the chambers measure breaks in X, Y, and Z planes. Typically data are reported for Horizontal beam breaks and sometimes for distance travelled and stereotypy in studies involving PCP.

Thus, Nelotanserin performs as an antagonist in cell-based 5HT2A signaling assays (βarrestin2 recruitment) and in mouse models of 5HT2A block (DOI-induced head twitch) and psychosis-like behavior (PCP-induced hyperlocomotion). In all three assays, nelotanserin acts in a manner similar to clozapine, a clinically relevant atypical antipsychotic. These data suggest that nelotanserin, and other 5HT2A serotonin receptor inverse agonists may prove useful in the treatment of psychotic symptoms.

In each of the embodiments described herein, the methods may comprise administering a therapeutically effective amount of a 5-HT_{2A} serotonin receptor inverse agonist.

In some embodiments, the 5-HT_{2A} serotonin receptor inverse agonist is selected from those disclosed in U.S. Pat. Nos. 8,754,238 and 9,434,692, each of which is hereby incorporated in its entirety for any purposes. In the descriptions of each group of compounds below, the respective substituents, R groups, numerical variables, apply to the description of each set of compounds, despite the fact that duplicate numbering may be used. The substituent lists, R groups, numerical variables etc. mirror the original patent disclosures. Thus, for example, R₁ will appear with respect to the compounds of both U.S. Pat. Nos. 8,754,238 and 9,434,692. With this clarification, it should be clear to those of ordinary skill in the art that the duplicate references are not indefinite and clearly are limited to the compounds with which they are associated.

In some embodiments, the 5-HT_{2A} serotonin receptor inverse agonist is selected from those disclosed in U.S. Pat. No. 8,754,238, hereby incorporated in its entirety for any purposes. Particularly, the 5-HT_{2A} serotonin receptor inverse agonist can be certain diaryl and arylheteroaryl urea derivatives as shown in Formula (I):

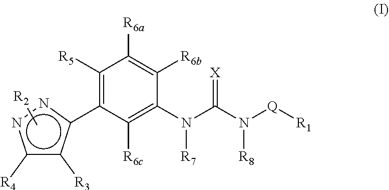

or a pharmaceutically acceptable salt, hydrate or solvate thereof; wherein R₁, R₂, R₃, R₄, R₅, R₆ₐ, R_{6b}, R_{6c}, R₇, R₈, X, and Q have the same definitions as described herein, supra and infra.

Some embodiments of the present invention encompass certain diaryl and arylheteroaryl urea derivatives as shown in the following Formula

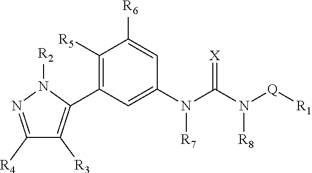

wherein:

i) R₁ is aryl or heteroaryl optionally substituted with R₉, R₁₀, R₁₁, R₁₂, R₁₃, R₁₄, and R₁₅ selected independently from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ di alkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol, nitro, phenoxy and phenyl, or two adjacent R₉, R₁₀, R₁₁, R₁₂, R₁₃, R₁₄, and R₁₅ together with the atoms to which they are attached form a C₅₋₇ cycloalkyl group or heterocyclic group each optionally substituted with F, Cl, or Br; and wherein each of said C₂₋₆ alkenyl, C₁₋₆ alkyl, C₂₋₆ alkynyl and phenyl groups can be optionally substituted with 1 to 5 substituents selected independently from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol and nitro;

ii) R₂ is selected from the group consisting of C₁₋₆ alkyl, C₂₋₆ alkenyl, C₂₋₆ alkynyl and C₃₋₇ cycloalkyl;

iii) R₃ is selected from the group consisting of H, C₂₋₆ alkenyl, C₁₋₆ alkyl, C₁₋₆ alkylcarboxamide, C₂₋₆ alkynyl, C₁₋₆ alkyl sulfonamide, carbo-C₁₋₆-alkoxy, carboxamide, carboxy, cyano, C₃₋₇ cycloalkyl, C₂₋₈ dialkylcarboxamide, halogen, heteroaryl and phenyl; and wherein each of said C₂₋₆ alkenyl, C₁₋₆ alkyl, C₂₋₆ alkynyl, C₁₋₆ alkylsulfonamide, C₃₋₇ cycloalkyl, heteroaryl and phenyl groups can be optionally substituted with 1 to 5 substituents selected independently from the group consisting of C₁₋₅ acyl, C₁₋₅ acyloxy, C₂₋₆ alkenyl, C₁₋₄ alkoxy, C₁₋₈ alkyl, C₁₋₆ alkylamino, C₂₋₈ dialkylamino, C₁₋₄ alkylcarboxamide, C₂₋₆ alkynyl, C₁₋₄ alkylsulfonamide, C₁₋₄ alkylsulfinyl, C₁₋₄ alkylsulfonyl, C₁₋₄ alkylthio, C₁₋₄ alkylureyl, amino, carbo-C₁₋₆-alkoxy, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ haloalkylthio, hydroxyl, nitro and sulfonamide;

iv) R₄ is selected from the group consisting of H, C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol, nitro and sulfonamide;

v) R₅ is selected from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol, nitro and sulfonamide, wherein said C₁₋₆ alkoxy group can be optionally substituted with 1 to 5 substituents selected independently from the group consisting of C₁₋₅ acyl, C₁₋₅ acyloxy, C₂₋₆ alkenyl, C₁₋₄ alkoxy, C₁₋₈ alkyl, C₁₋₆ alkylamino, C₂₋₈ dialkylamino, C₁₋₄ alkylcarboxamide, C₂₋₆ alkynyl, C₁₋₄ alkylsulfonamide, C₁₋₄ alkylsulfinyl, C₁₋₄ alkylsulfonyl, C₁₋₄ alkylthio, C₁₋₄ alkylureyl, amino, carbo-C₁₋₆-alkoxy, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ haloalkylthio, hydroxyl, nitro and phenyl, and wherein said phenyl is optionally substituted with 1 to 5 halogen atoms;

vi) R₆ is selected from the group consisting of H, C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol, nitro and sulfonamide;

vii) R₇ and R₈ are independently H or C₁₋₈ alkyl;

viii) X is O or S; and

ix) Q is C₁₋₃ alkylene optionally substituted with 1 to 4 substituents selected from the group consisting of C₁₋₃ alkyl, C₁₋₄ alkoxy, carboxy, cyano, C₁₋₃ haloalkyl, halogen and oxo; or Q is a bond; or a pharmaceutically acceptable salt, hydrate or solvate thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable subcombination.

As used herein, “substituted” indicates that at least one hydrogen atom of the chemical group is replaced by a non-hydrogen substituent or group, the non-hydrogen substituent or group can be monovalent or divalent. When the substituent or group is divalent, then it is understood that this group is further substituted with another substituent or group. When a chemical group herein is “substituted” it may have up to the full valance of substitution; for example, a methyl group can be substituted by 1, 2, or 3 substituents, a methylene group can be substituted by 1 or 2 substituents, a phenyl group can be substituted by 1, 2, 3, 4, or 5 substituents, a naphthyl group can be substituted by 1, 2, 3, 4, 5, 6, or 7 substituents and the like. Likewise, “substituted with one or more substituents” refers to the substitution of a group with one substituent up to the total number of substituents physically allowed by the group. Further, when a group is substituted with more than one group they can be identical or they can be different.

Compounds of the invention can also include tautomeric forms, such as keto-enol tautomers, and the like. Tautomeric forms can be in equilibrium or sterically locked into one form by appropriate substitution. It is understood that the various tautomeric forms are within the scope of the compounds of the present invention.

Compounds of the invention can also include all isotopes of atoms occurring in the intermediates and/or final compounds. Isotopes include those atoms having the same atomic number but different mass numbers. For example, isotopes of hydrogen include deuterium and tritium.

It is understood and appreciated that compounds of the present invention may have one or more chiral centers, and therefore can exist as enantiomers and/or diastereomers. The invention is understood to extend to and embrace all such enantiomers, diastereomers and mixtures thereof, including but not limited, to racemates. Accordingly, some embodiments of the present invention pertain to compounds of the present invention that are R enantiomers. Further, some embodiments of the present invention pertain to compounds of the present invention that are S enantiomers. In examples where more than one chiral center is present, then, some embodiments of the present invention include compounds that are RS or SR enantiomers. In further embodiments, compounds of the present invention are RR or SS enantiomers. It is understood that compounds of the present invention are intended to represent all individual enantiomers and mixtures thereof, unless stated or shown otherwise.

In some embodiments, R₁ is aryl or heteroaryl each optionally substituted with R₉, R₁₀, R₁₁, R₁₂, R₁₃, R₁₄, and R₁₅ each selected independently from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkyl sulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, C₁₋₆ alkylimino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, heterocyclic, hydroxyl, thiol, nitro, phenoxy and phenyl, wherein said C₂₋₆ alkenyl, C₁₋₆ alkyl, C₂₋₆ alkynyl, C₁₋₆ alkylamino, C₁₋₆ alkylimino, C₂₋₈ dialkylamino, heterocyclic, and phenyl are each optionally substituted with 1 to 5 substituents selected independently from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol and nitro;

Some embodiments of the present invention pertain to compounds wherein R₁ is phenyl or naphthyl each optionally substituted with R₉, R₁₀, R₁₁, R₁₂, R₁₃, R₁₄, and R₁₅ each selected independently from the group consisting of C₁₋₆ acyl, C₁₋₆ alkoxy, C₁₋₆ alkyl, C₁₋₆ alkylsulfonyl, amino, C₁₋₆ alkylamino, C₂₋₈ dialkylamino, C₁₋₆ alkylimino, carbo-C₁₋₆-alkoxy, carboxamide, carboxy, cyano, C₃₋₇ cycloalkyl, halogen, C₁₋₆ haloalkoxy, C₁₋₆ haloalkyl, heterocyclic, hydroxyl, nitro, and phenyl, or two adjacent R₉, R₁₀, R₁₁, R₁₂, R₁₃, R₁₄, and R₁₅ together with the atoms to which they are attached form a C₅₋₇ cycloalkyl group or heterocyclic group each optionally substituted with F; and wherein said C₁₋₆ alkyl, C₁₋₆ alkylimino, and heterocyclic are each optionally substituted with 1 to 5 substituents selected independently from the group consisting of C₁₋₆ acyl, C₁₋₆ alkoxy, C₁₋₆ alkyl, C₁₋₆ alkylsulfonyl, amino, C₁₋₆ alkylamino, C₂₋₈ dialkylamino, carboxamide, cyano, C₃₋₇ cycloalkyl, halogen, C₁₋₆ haloalkoxy, C₁₋₆ haloalkyl, and hydroxyl.

Some embodiments of the present invention pertain to compounds wherein R₁ is phenyl optionally substituted with R₉, R₁₀, R₁₁, R₁₂, and R₁₃ each selected independently from the group consisting of C₁₋₆ acyl, C₁₋₆ alkoxy, C₁₋₆ alkyl, C₁₋₆ alkylsulfonyl, amino, C₁₋₆ alkylamino, C₂₋₈ dialkylamino, C₁₋₆ alkylimino, carbo-C₁₋₆-alkoxy, carboxamide, carboxy, cyano, C₃₋₇ cycloalkyl, halogen, C₁₋₆ haloalkoxy, C₁₋₆ haloalkyl, heterocyclic, hydroxyl, nitro, and phenyl, or two adjacent R₉, R₁₀, R₁₁, R₁₂, and R₁₃ together with the atoms to which they are attached form a C₅₋₇ cycloalkyl group or heterocyclic group each optionally substituted with F; and wherein said C₁₋₆ alkyl, C₁₋₆ alkylimino, and heterocyclic are each optionally substituted with 1 to 5 substituents selected independently from the group consisting of C₁₋₆ acyl, C₁₋₆ alkoxy, C₁₋₆ alkyl, C₁₋₆ alkylsulfonyl, amino, C₁₋₆ alkylamino, C₂₋₈ dialkylamino, carboxamide, cyano, C₃₋₇ cycloalkyl, halogen, C₁₋₆ haloalkoxy, C₁₋₆ haloalkyl, and hydroxyl.

Some embodiments of the present invention pertain to compounds wherein R₁ is phenyl or naphthyl each optionally substituted with R₉, R₁₀, R₁₁, R₁₂, R₁₃, R₁₄, and R₁₅ each selected independently from the group consisting of C₁₋₆ acyl, C₁₋₆ alkoxy, C₁₋₆ alkyl, amino, C₁₋₆ alkylamino, C₂₋₈ dialkylamino, C₁₋₆ alkylimino, cyano, halogen, C₁₋₆ haloalkoxy, C₁₋₆ haloalkyl, heterocyclic, hydroxyl, nitro, and phenyl, or two adjacent R₉, R₁₀, R₁₁, R₁₂, R₁₃, R₁₄, and R₁₅ together with the atoms to which they are attached form a C₅₋₇ cycloalkyl group or heterocyclic group each optionally substituted with F; and wherein said C₁₋₆ alkyl, C₁₋₆ alkylimino, and heterocyclic are each optionally substituted with 1 to 5 substituents selected independently from the group consisting of C₁₋₆ alkyl, amino, C₁₋₆ alkylamino, C₂₋₈ dialkylamino, and hydroxyl.

Some embodiments of the present invention pertain to compounds wherein R₁ is phenyl optionally substituted with R₉, R₁₀, R₁₁, R₁₂, and R₁₃ each selected independently from the group consisting of C₁₋₆ acyl, C₁₋₆ alkoxy, C₁₋₆ alkyl, amino, C₁₋₆ alkylamino, C₂₋₈ dialkylamino, C₁₋₆ alkylimino, cyano, halogen, C₁₋₆ haloalkoxy, C₁₋₆ haloalkyl, heterocyclic, hydroxyl, nitro, and phenyl, or two adjacent R₉, R₁₀, R₁₁, R₁₂, and R₁₃ together with the atoms to which they are attached form a C₅₋₇ cycloalkyl group or heterocyclic group each optionally substituted with F; and wherein said C₁₋₆ alkyl, C₁₋₆ alkylimino, and heterocyclic are each optionally substituted with 1 to 5 substituents selected independently from the group consisting of C₁₋₆ alkyl, amino, C₁₋₆ alkylamino, C₂₋₈ dialkylamino, and hydroxyl.

Some embodiments of the present invention pertain to compounds wherein R₁ is phenyl or naphthyl optionally substituted with R₉, R₁₀, R₁₁, R₁₂, R₁₃, R₁₄, and R₁₅ each selected independently from the group consisting of —C(O)CH₃, —OCH₃, —CH₃, —CH(CH₃)₂, —CH(OH)CH₃, —N(CH₃)₂, (2-dimethylamino-ethyl)-methyl-amino [i.e., —N(CH₃)CH₂CH₂N(CH₃)₂], (3-dimethylamino-propyl)-methyl-amino [i.e., —N(CH₃)CH₂CH₂CH₂N(CH₃)₂], —C(═NOH)CH₃, cyano, —F, —Cl, —Br, —OCF₃, —CF₃, 4-methyl-piperazin-1-yl, morpholin-4-yl, 4-methyl-piperidin-1-yl, hydroxyl, nitro, and phenyl.

Some embodiments of the present invention pertain to compounds wherein R₁ is phenyl optionally substituted with R₉, R₁₀, R₁₁, R₁₂, and R₁₃, R₁₄ each selected independently from the group consisting of —C(O)CH₃, —OCH₃, —CH₃, —CH(CH₃)₂, —CH(OH)CH₃, —N(CH₃)₂, (2-dimethylamino-ethyl)-methyl-amino [i.e., —N(CH₃)CH₂CH₂N(CH₃)₂], (3-dimethylamino-propyl)-methyl-amino [i.e., —N(CH₃)CH₂CH₂CH₂N(CH₃)₂], —C(═NOH)CH₃, cyano, —F, —Cl, —Br, —OCF₃, —CF₃, 4-methyl-piperazin-1-yl, morpholin-4-yl, 4-methyl-piperidin-1-yl, hydroxyl, nitro, and phenyl.

Some embodiments of the present invention pertain to compounds wherein R₁ is phenyl or naphthyl optionally substituted with R₉, R₁₀, R₁₁, R₁₂, R₁₃, R₁₄, and R₁₅ each selected independently from the group consisting of —OCH₃, —CH₃, cyano, —F, —Cl, —Br, —OCF₃, and —CF₃.

Some embodiments of the present invention pertain to compounds wherein R₁ is phenyl optionally substituted with R₉, R₁₀, R₁₁, R₁₂, and R₁₃ each selected independently from the group consisting of —OCH₃, —CH₃, cyano, —F, —Cl, —Br, —OCF₃, and —CF₃.

Some embodiments of the present invention pertain to compounds wherein R₁ is phenyl and can be represented by the Formula shown below:

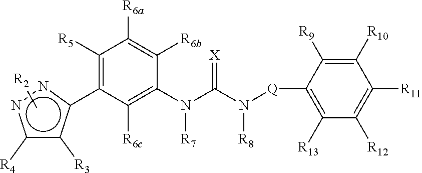

wherein each variable in the above formula has the same meaning as described herein, supra and infra. In some embodiments, R₇ and R₈ are both —H, Q is a bond, and X is O.

Some embodiments of the present invention pertain to compounds wherein R₁ is phenyl and can be represented by Formula (Ia) as shown below:

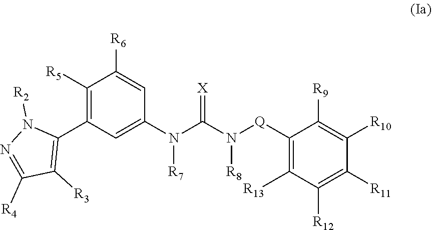

wherein:

R₉ to R₁₃ substituents are each selected independently from the group consisting of H, C₁₋₆ acyl, C₁₋₆ acyloxy, C₁₋₆ alkoxy, C₁₋₆ alkyl, C₁₋₆ alkylcarboxamide, C₁₋₆ alkylsulfonamide, C₁₋₆ alkylsulfinyl, C₁₋₆ alkylsulfonyl, C₁₋₆ alkylthio, amino, C₁₋₆ alkylamino, C₂₋₈ dialkylamino, carbo-C₁₋₆-alkoxy, carboxamide, carboxy, cyano, halogen, C₁₋₆ haloalkoxy, C₁₋₆ haloalkyl, hydroxyl, nitro and phenyl, or two adjacent substituents together with the phenyl form a C₅₋₇ cycloalkyl optionally comprising 1 to 2 oxygen atoms; and wherein each said C₁₋₆ alkyl and phenyl groups can be optionally substituted with 1 to 5 substituents selected independently from the group consisting of C₁₋₆ alkoxy, C₁₋₆ alkyl, amino, cyano, halogen, C₁₋₆ haloalkoxy, C₁₋₆ haloalkyl, hydroxyl and nitro.

In some embodiments, R₁ is phenyl optionally substituted with R₉ to R₁₃ substituents selected independently from the group consisting of C₁₋₆ acyl, C₁₋₆ alkoxy, C₁₋₆ alkyl, cyano, halogen, C₁₋₆ haloalkoxy, C₁₋₆ haloalkyl, nitro and phenyl; and wherein said phenyl can be optionally substituted with 1 to 5 substituents selected independently from the group consisting of C₁₋₆ alkoxy, C₁₋₆ alkyl, cyano, halogen, C₁₋₆ haloalkoxy, C₁₋₆ haloalkyl and nitro.

In some embodiments, R₁ is phenyl optionally substituted with R₉ to R₁₃ substituents selected independently from the group consisting of C₁₋₆ acyl, C₁₋₆ alkoxy, C₁₋₆ alkyl, cyano, halogen, C₁₋₆ haloalkoxy, C₁₋₆ haloalkyl, nitro and phenyl.

In some embodiments, R₁ is phenyl optionally substituted with R₉ to R₁₃ substituents selected independently from the group consisting of —C(O)CH₃, —C(O)CH₂CH₃, —C(O)CH(CH₃)₂, —C(O)CH₂CH₂CH₃, —C(O)CH₂CH(CH₃)₂, —OCH₃, —OCH₂CH₃, —OCH(CH₃)₂, —OCH₂CH₂CH₃, —OCH₂CH(CH₃)₂, —CH₃, —CH₂CH₃, —CH(CH₃)₂, —CH₂CH₂CH₃, —CH₂CH(CH₃)₂, —CH₂CH₂CH₂CH₃, cyano, F, Cl, Br, I, —OCF₃, —OCHF₂, —OCFH₂, —OCF₂CF₃, —OCH₂CF₃, —CF₃, —CHF₂, —CFH₂, —CF₂CF₃, —CH₂CF₃, nitro and phenyl.

In some embodiments, R₁ is phenyl optionally substituted with R₉ to R₁₃ substituents are each selected independently from the group consisting of —C(O)CH₃, —OCH₃, —CH₃, —CH(CH₃)₂, —CH(OH)CH₃, —N(CH₃)₂, (2-dimethylamino-ethyl)-methyl-amino, (3-dimethylamino-propyl)-methyl-amino, —C(═NOH)CH₃, cyano, —F, —Cl, —Br, —OCF₃, —CF₃, 4-methyl-piperazin-1-yl, morpholin-4-yl, 4-methyl-piperidin-1-yl, hydroxyl, nitro, and phenyl.

In some embodiments, R₁ is phenyl optionally substituted with R₉, R₁₀, R₁₁, R₁₂ and R₁₃ substituents selected independently from the group consisting of —C(O)CH₃, —OCH₃, —CH₃, cyano, —F, —Cl, —Br, —OCF₃, —CF₃, nitro and phenyl.

Some embodiments of the present invention pertain to compounds wherein R₁ is naphthyl optionally substituted with R₉ R₁₀ R₁₁ R₁₂ R₁₃ R₁₄ and R₁₅ substituents selected independently from the group consisting of C₁₋₆ acyl, C₁₋₆ acyloxy, C₁₋₆ alkoxy, C₁₋₆ alkyl, C₁₋₆ alkylcarboxamide, C₁₋₆ alkyl sulfonamide, C₁₋₆ alkylsulfinyl, C₁₋₆ alkylsulfonyl, C₁₋₆ alkylthio, amino, C₁₋₆ alkylamino, C₂₋₈ dialkylamino, carbo-C₁₋₆-alkoxy, carboxamide, carboxy, cyano, halogen, C₁₋₆ haloalkoxy, C₁₋₆ haloalkyl, hydroxyl and nitro; and wherein said C₁₋₆ alkyl can be optionally substituted with 1 to 5 substituents selected independently from the group consisting of C₁₋₆ alkoxy, C₁₋₆ alkyl, amino, cyano, halogen, C₁₋₆ haloalkoxy, C₁₋₆ haloalkyl, hydroxyl and nitro.

In some embodiments, R₁ is naphthyl optionally substituted with R₉, R₁₀, R₁₁, R₁₂, R₁₃, R₁₄ and R₁₅ substituents selected independently from the group consisting of C₁₋₆ acyl, C₁₋₆ alkoxy, C₁₋₆ alkyl, cyano, halogen, C₁₋₆ haloalkoxy, C₁₋₆ haloalkyl and nitro.

In some embodiments, R₁ is naphthyl optionally substituted with R₉, R₁₀, R₁₁, R₁₂, R₁₃, R₁₄ and R₁₅ substituents selected independently from the group consisting of —C(O)CH₃, —C(O)CH₂CH₃, —C(O)CH(CH₃)₂, —C(O)CH₂CH₂CH₃, —C(O)CH₂CH(CH₃)₂, —OCH₃, —OCH₂CH₃, —OCH(CH₃)₂, —OCH₂CH₂CH₃, —OCH₂CH(CH₃)₂, —CH₃, —CH₂CH₃, —CH(CH₃)₂, —CH₂CH₂CH₃, —CH₂CH(CH₃)₂, —CH₂CH₂CH₂CH₃, cyano, —F, —Cl, —Br, —I, —OCF₃, —OCHF₂, —OCFH₂, —OCF₂CF₃, —OCH₂CF₃, —CF₃, —CFH₂, —CF₂CF₃, —CH₂CF₃ and nitro.

In some embodiments, R₁ is naphthyl optionally substituted with R₉, R₁₀, R₁₁, R₁₂, R₁₃, R₁₄ and R₁₅ substituents selected independently from the group consisting of —C(O)CH₃, —C(O)CH₂CH₃, —C(O)CH(CH₃)₂, —C(O)CH₂CH₂CH₃, —C(O)CH₂CH(CH₃)₂, —OCH₃, —OCH₂CH₃, —OCH(CH₃)₂, —OCH₂CH₂CH₃, —OCH₂CH(CH₃)₂, —CH₃, —CH₂CH₃, —CH(CH₃)₂, —CH₂CH₂CH₃, —CH₂CH(CH₃)₂, —CH₂CH₂CH₂CH₃, cyano, —F, —Cl, —Br, —I, —OCF₃, —OCHF₂, —OCFH₂, —OCF₂CF₃, —OCH₂CF₃, —CF₃, —CHF₂, —CFH₂, —CF₂CF₃, —CH₂CF₃ and nitro.

In some embodiments, R₁ is naphthyl optionally substituted with R₉, R₁₀, R₁₁, R₁₂, R₁₃, R₁₄ and R₁₅ substituents selected independently from the group consisting of —C(O)CH₃, —OCH₃, —CH₃, cyano, —F, —Cl, —Br, —OCF₃, —CF₃ and nitro.

Some embodiments of the present invention pertain to compounds wherein R₁ is heteroaryl optionally substituted with R₉, R₁₀, R₁₁, R₁₂, and R₁₃ each selected independently from the group consisting of C₁₋₆ acyl, C₁₋₆ alkoxy, C₁₋₆ alkyl, amino, C₁₋₆ alkylamino, C₂₋₈ dialkylamino, C₁₋₆ alkylimino, cyano, halogen, C₁₋₆ haloalkoxy, C₁₋₆ haloalkyl, heterocyclic, hydroxyl, nitro, and phenyl, or two adjacent R₉, R₁₀, R₁₁, R₁₂, R₁₃, R₁₄, and R₁₅ together with the atoms to which they are attached form a C₅₋₇ cycloalkyl group or heterocyclic group each optionally substituted with F; and wherein said C₁₋₆ alkyl, C₁₋₆ alkylimino, and heterocyclic are each optionally substituted with 1 to 5 substituents selected independently from the group consisting of C₁₋₆ alkyl, amino, C₁₋₆ alkylamino, C₂₋₈ dialkylamino, and hydroxyl.

Some embodiments of the present invention pertain to compounds wherein R₁ is heteroaryl optionally substituted with R₉, R₁₀, R₁₁, R₁₂, and R₁₃ each selected independently from the group consisting of —C(O)CH₃, —OCH₃, —CH₃, —CH(CH₃)₂, —CH(OH)CH₃, —N(CH₃)₂, (2-dimethylamino-ethyl)-methyl-amino, (3-dimethylamino-propyl)-methyl-amino, —C(═NOH)CH₃, cyano, —F, —Cl, —Br, —OCF₃, —CF₃, 4-methyl-piperazin-1-yl, morpholin-4-yl, 4-methyl-piperidin-1-yl, hydroxyl, nitro, and phenyl.

Some embodiments of the present invention pertain to compounds wherein R₁ is heteroaryl optionally substituted with R₉, R₁₀, R₁₁, R₁₂, and R₁₃ each selected independently from the group consisting of —OCH₃, —CH₃, cyano, —F, —Cl, —Br, —OCF₃, and —CF₃.

Some embodiments of the present invention pertain to compounds wherein R₁ is heteroaryl optionally substituted with R₉, R₁₀, R₁₁, R₁₂, and R₁₃ each selected independently from the group consisting of C₁₋₆ acyl, C₁₋₆ acyloxy, C₁₋₆ alkoxy, C₁₋₆ alkyl, C₁₋₆ alkylcarboxamide, C₁₋₆ alkyl sulfonamide, C₁₋₆ alkylsulfinyl, C₁₋₆ alkylsulfonyl, C₁₋₆ alkylthio, amino, C₁₋₆ alkylamino, C₂₋₈ dialkylamino, carbo-C₁₋₆-alkoxy, carboxamide, carboxy, cyano, halogen, C₁₋₆ haloalkoxy, C₁₋₆ haloalkyl, hydroxyl, nitro and phenyl, or two adjacent R₉, R₁₀, R₁₁, R₁₂, R₁₃, R₁₄, and R₁₅ together with the atoms to which they are attached form a C₅₋₇ cycloalkyl group or heterocyclic group; and wherein each of said C₁₋₆ alkyl and phenyl groups can be optionally substituted with 1 to 5 substituents selected independently from the group consisting of C₁₋₆ alkoxy, C₁₋₆ alkyl, amino, cyano, halogen, C₁₋₆ haloalkoxy, C₁₋₆ haloalkyl, hydroxyl and nitro.

In some embodiments, R₁ is heteroaryl optionally substituted with R₉, R₁₀, R₁₁, R₁₂ and R₁₃ each selected independently from the group consisting of C₁₋₆ acyl, C₁₋₆ alkoxy, C₁₋₆ alkyl, cyano, halogen, C₁₋₆ haloalkoxy, C₁₋₆ haloalkyl, nitro and phenyl; and wherein said phenyl can be optionally substituted with 1 to 5 substituents selected independently from the group consisting of C₁₋₆ alkoxy, C₁₋₆ alkyl, cyano, halogen, C₁₋₆ haloalkoxy, C₁₋₆ haloalkyl and nitro.

In some embodiments, R₁ is heteroaryl optionally substituted with R₉, R₁₀, R₁₁, R₁₂ and R₁₃ each selected independently from the group consisting of C₁₋₆ acyl, C₁₋₆ alkoxy, C₁₋₆ alkyl, cyano, halogen, C₁₋₆ haloalkoxy, C₁₋₆ haloalkyl, nitro and phenyl.

In some embodiments, R₁ is heteroaryl optionally substituted with R₉, R₁₀, R₁₁, R₁₂, and R₁₃ each selected independently from the group consisting of —C(O)CH₃, —C(O)CH₂CH₃, —C(O)CH(CH₃)₂, —C(O)CH₂CH₂CH₃, —C(O)CH₂CH(CH₃)₂, —OCH₃, —OCH₂CH₃, —OCH(CH₃)₂, —OCH₂CH₂CH₃, —OCH₂CH(CH₃)₂, —CH₃, —CH₂CH₃, —CH(CH₃)₂, —CH₂CH₂CH₃, —CH₂CH(CH₃)₂, —CH₂CH₂CH₂CH₃, cyano, —F, —Cl, —Br, —I, —OCF₃, —OCHF₂, —OCFH₂, —OCF₂CF₃, —OCH₂CF₃, —CF₃, —CHF₂, —CF₂CF₃, —CH₂CF₃, nitro and phenyl.

In some embodiments, R₁ is heteroaryl optionally substituted with R₉, R₁₀, R₁₁, R₁₂, and R₁₃ each selected independently from the group consisting of —C(O)CH₃, —OCH₃, —CH₃, cyano, —F, —Cl, —Br, —OCF₃, —CF₃, nitro and phenyl. In some embodiments, R₁ is heteroaryl optionally substituted with R₉, R₁₀, R₁₁, R₁₂, and R₁₃ selected independently from the group consisting of H, —C(O)CH₃, —OCH₃, —CH₃, cyano, —F, —Cl, —Br, —OCF₃, —CF₃, nitro and phenyl.

In some embodiments R₁ is heteroaryl having 5-atoms in the aromatic ring examples of which are represented by the following formulae:

TABLE 2

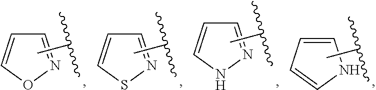

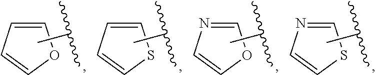

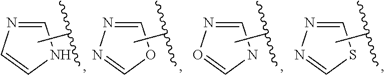

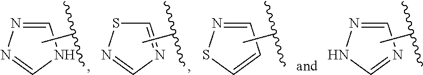

wherein the 5-membered heteroaryl is bonded at any available position of the ring, for example, a imidazolyl ring can be bonded at one of the ring nitrogens (i.e., imidazol-1-yl group) or at one of the ring carbons (i.e., imidazol-2-yl, imidazol-4-yl or imidazol-5-yl group).

In some embodiments, R₁ is a 6-membered heteroaryl, for example, a 6-membered heteroaryl as shown in TABLE 3:

TABLE 3

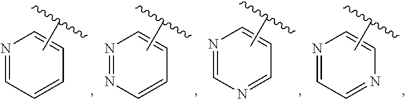

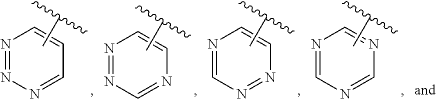

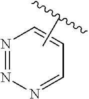

wherein the heteroaryl group is bonded at any ring carbon. In some embodiments, R₁ is selected from the group consisting of pyridinyl, pyridazinyl, pyrimidinyl and pyrazinyl. In some embodiments, R₁ is pyridinyl.

In some embodiments R₁ is a heteroaryl, for example but not limited to those shown in TABLE 2 and 3, optionally substituted with 1 to 3 substituents selected from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkyl sulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol, nitro, phenoxy and phenyl; and wherein each of said C₂₋₆ alkenyl, C₁₋₆ alkyl, C₂₋₆ alkynyl and phenyl groups can be optionally substituted with 1 to 5 substituents selected independently from the group consisting of C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol and nitro.

Some embodiments pertain to compounds wherein R₂ is H or C₁₋₆ alkyl.

Some embodiments pertain to compounds wherein R₂ is C₁₋₆ alkyl. In some embodiments, R₂ is selected from the group consisting of —CH₃, —CH₂CH₃, —CH(CH₃)₂, —CH₂CH₂CH₃, —CH₂CH(CH₃)₂ and —CH₂CH₂CH₂CH₃. In some embodiments, R₂ is —CH₃ or —CH(CH₃)₂.

Some embodiments can be represented by Formulae (Ib) and (Ic) respectively as shown below:

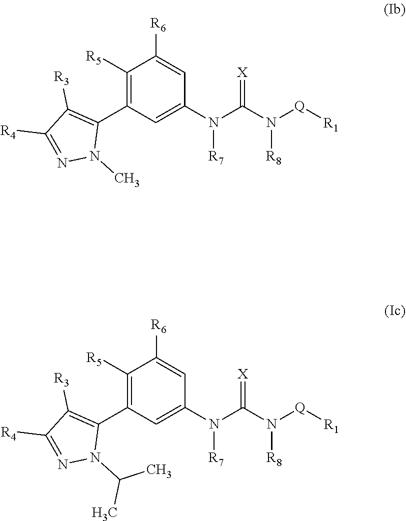

wherein each variable in Formulae (Ib) and (Ic) has the same meaning as described herein, supra and infra.

Some embodiments pertain to compounds wherein R₂ is H.

It is understood that when R₂ is H, then tautomers are possible. It is well understood and appreciated in the art that pyrazoles can exist in various tautomeric forms. Two possible tautomeric forms are illustrated below:

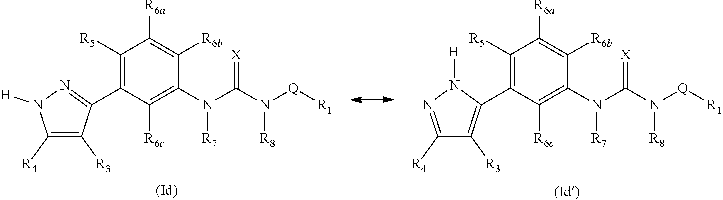

It is further understood that tautomeric forms can also have corresponding nomenclature for each represented tautomer, for example, Formula (Id) and Formula (Id′) can be represented by the general chemical names 1H-pyrazol-3-yl and 2H-pyrazole-3-yl respectively. Therefore, the present invention includes all tautomers and the various nomenclature designations.

Some embodiments pertain to compounds wherein R₂ is C₂₋₆ alkenyl. In some embodiments, R₂ is —CH₂CH═CH₂.

Some embodiments pertain to compounds wherein R₂ is C₂₋₆ alkynyl.

Some embodiments pertain to compounds wherein R₂ is C₃₋₇ cycloalkyl. In some embodiments, R₂ is cyclopropyl.

Some embodiments pertain to compounds wherein R₃ is selected from the group consisting of H, C₂₋₆ alkenyl, C₁₋₆ alkyl, C₁₋₆ alkylcarboxamide, C₂₋₆ alkynyl, carbo-C₁₋₆-alkoxy, carboxamide, carboxy, cyano, C₃₋₇ cycloalkyl, halogen, heteroaryl or phenyl; and wherein each of said C₂₋₆ alkenyl, C₁₋₆ alkyl, C₂₋₆ alkynyl, heteroaryl and phenyl groups can be optionally substituted with 1 to 5 substituents selected independently from the group consisting of C₁₋₆ alkylamino, C₂₋₈ dialkylamino, C₂₋₆ alkenyl, C₁₋₄ alkoxy, C₁₋₈ alkyl, C₂₋₆ alkynyl, amino, halogen, C₁₋₄ haloalkoxy and hydroxyl.

In some embodiments, R₃ is selected from the group consisting of H, C₂₋₆ alkenyl, C₁₋₆ alkyl, C₂₋₆ alkynyl, carbo-C₁₋₆-alkoxy, carboxy, cyano, C₃₋₇ cycloalkyl, halogen, heteroaryl or phenyl; and wherein each of said C₂₋₆ alkenyl, C₁₋₆ alkyl, C₂₋₆ alkynyl and phenyl groups can be optionally substituted with 1 to 5 substituents selected independently from the group consisting of C₂₋₈ dialkylamino, C₂₋₆ alkenyl, C₁₋₄ alkoxy, C₂₋₆ alkynyl, halogen, C₁₋₄ haloalkoxy and hydroxyl.

In some embodiments, R₃ is selected from the group consisting of H, —CH═CH₂, —CH₃, —CH₂CH₃, —CH(CH₃)₂, —CH₂CH₂CH₃, —CH₂CH(CH₃)₂, —CH₂CH₂CH₂CH₃, —C(O)OCH₃, —C(O)OCH₂CH₃, carboxy, cyano, cyclopropyl, F, Cl, Br, I, thiophen-2-yl, thiophen-3-yl, phenyl, —CH₂CH₂N(CH₃)₂, 2-methoxyphenyl, 3-methoxyphenyl, 4-methoxyphenyl, —CH═CH—C≡CH, 4-fluorophenyl, 4-trifluoromethoxyphenyl, —CH₂OH and —CH₂CH₂OH.

Some embodiments pertain to compounds wherein R₃ is H or halogen.

In some embodiments, R₃ is H, F, Cl or Br.

Some embodiments pertain to compounds of Formula (Ie) as shown below:

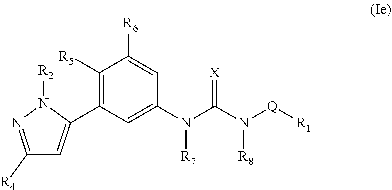

wherein each variable in Formula (Ie) has the same meaning as described herein, supra and infra.

Some embodiments pertain to compounds of Formula (If) as shown below:

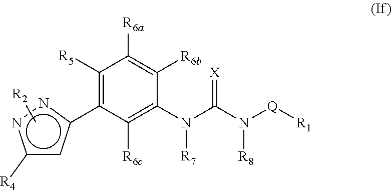

wherein each variable in Formula (If) has the same meaning as described herein, supra and infra.

Some embodiments pertain to compounds of Formula (Ig) as shown below:

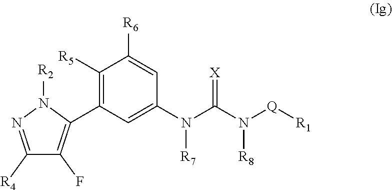

wherein each variable in Formula (Ig) has the same meaning as described herein, supra and infra.

Some embodiments pertain to compounds of Formula (Ih) as shown below:

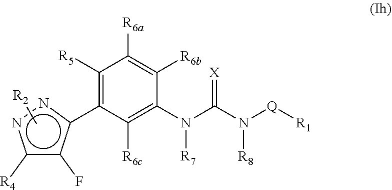

wherein each variable in Formula (Ih) has the same meaning as described herein, supra and infra.

Some embodiments pertain to compounds of Formula (Ii) as shown below:

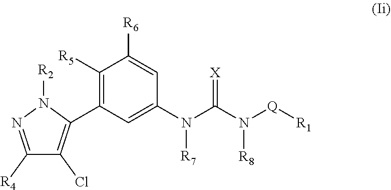

wherein each variable in Formula (Ii) has the same meaning as described herein, supra and infra.

Some embodiments pertain to compounds of Formula (Ij) as shown below:

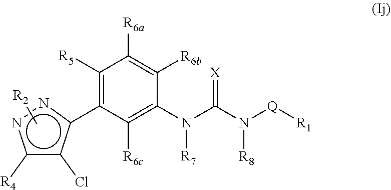

wherein each variable in Formula (Ij) has the same meaning as described herein, supra and infra.

Some embodiments pertain to compounds of Formula (Ik) as shown below:

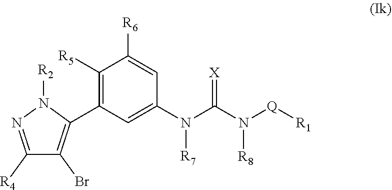

wherein each variable in Formula (Ik) has the same meaning as described herein, supra and infra.

Some embodiments pertain to compounds of Formula (Ik′) as shown below:

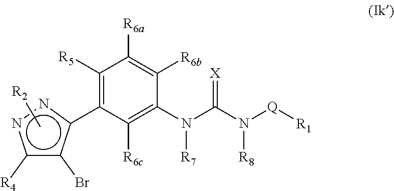

wherein each variable in Formula (Ik′) has the same meaning as described herein, supra and infra.

Some embodiments pertain to compounds wherein R₄ is selected from the group consisting of H, C₁₋₆ alkyl and C₁₋₆ haloalkyl.

In some embodiments, R₄ is selected from the group consisting of H, —CH₃, —CH₂CH₃, —CH(CH₃)₂, —CH₂CH₂CH₃, —CH₂CH(CH₃)₂, —CH₂CH₂CH₂CH₃, —CF₃, —CHF₂, —CFH₂, —CF₂CF₃ and —CH₂CF₃.

In some embodiments, R₄ is selected from the group consisting of H or —CF₃.

Some embodiments can be represented by Formulae (Im) and (In) as shown below:

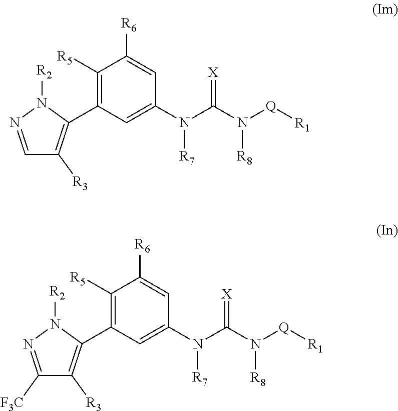

wherein each variable in Formulae (Im) and (In) has the same meaning as described herein, supra and infra.

Some embodiments can be represented by Formulae (Io) and (Io′) as shown below:

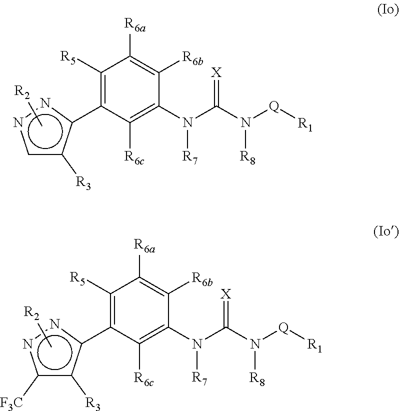

wherein each variable in Formulae (Io) and (Io′) has the same meaning as described herein, supra and infra.

Some embodiments pertain to compounds wherein R₅ is selected from the group consisting of C₁₋₆ alkoxy, C₁₋₆ alkylthio, amino, C₁₋₆ alkylamino, C₂₋₈ dialkylamino, halogen, C₁₋₆ haloalkoxy, and hydroxyl, wherein said C₁₋₆ alkoxy group can be optionally substituted with 1 to 5 substituents selected independently from the group consisting of amino, C₁₋₆ alkylamino, C₂₋₈ dialkylamino, amino, carbo-C₁₋₆-alkoxy, carboxamide, carboxy, cyano, halogen, and phenyl, and wherein said amino and phenyl are each optionally substituted with 1 to 5 further substituents selected from the group consisting of halogen and carbo-C₁₋₆-alkoxy.

Some embodiments pertain to compounds wherein R₅ is C₁₋₆ alkoxy, or hydroxyl, wherein said C₁₋₆ alkoxy group can be optionally substituted with 1 to 5 substituents selected independently from the group consisting of C₁₋₄ alkoxy, C₁₋₆ alkylamino, C₂₋₈ dialkylamino, alkylsulfinyl, C₁₋₄ alkylsulfonyl, C₁₋₄ alkylthio, amino, halogen, C₁₋₄ haloalkoxy, C₁₋₄ haloalkyl, C₁₋₄ haloalkylsulfinyl, C₁₋₄ haloalkylsulfonyl, C₁₋₄ haloalkylthio, hydroxyl and phenyl, and wherein said phenyl is optionally substituted with 1 to 5 halogen atoms.

Some embodiments pertain to compounds wherein R₅ is selected from the group consisting of C₁₋₆ alkoxy, C₁₋₆ haloalkoxy, and hydroxyl, wherein said C₁₋₆ alkoxy group can be optionally substituted with 1 to 5 substituents selected independently from the group consisting of amino, C₂₋₈ dialkylamino, carboxy, and phenyl, and wherein said amino and phenyl are each optionally substituted with 1 to 5 further substituents selected from the group consisting of halogen and carbo-C₁₋₆-alkoxy.

In some embodiments, R₅ is C₁₋₆ alkoxy, or hydroxyl, and wherein said C₁₋₆ alkoxy group can be optionally substituted with 1 to 5 substituents selected independently from the group consisting of C₁₋₄ alkoxy, C₁₋₆ alkylamino, C₂₋₈ dialkylamino, amino, C₁₋₄ haloalkoxy, hydroxyl and phenyl, wherein said phenyl is optionally substituted with 1 to 5 halogen atoms.

Some embodiments pertain to compounds wherein R₅ is selected from the group consisting of —OCH₃, —OCH₂CH₃, —OCH(CH₃)₂, —OCF₃, hydroxyl, benzyloxy, 4-chloro-benzyloxy, phenethyloxy, 2-dimethyl amino-ethoxy [i.e., —OCH₂CH₂N(CH₃)₂], 3-dimethylamino-propoxy [i.e., —OCH₂CH₂CH₂N(CH₃)₂], carboxymethoxy [i.e., —OCHC(O)OH], and 2-tert-butoxycarbonylamino-ethoxy [i.e., —OCH₂CH₂NHC(O)OC(CH₃)₃].

In some embodiments, R₅ is selected from the group consisting of —OCH₃, —OCH₂CH₃, —OCH(CH₃)₂, —OCH₂CH₂CH₃, —OCH₂CH(CH₃)₂, hydroxyl, —OCH₂CH₂OH, —OCH₂CH₂OCH₃, —OCH₂CH₂OCH₂CH₃, —OCH₂CH₂OCH(CH₃)₂, —OCH₂CH₂OCH₂CH₂CH₃, —OCH₂CH₂OCH₂CH(CH₃)₂, —OCH₂CH₂NH₂, —OCH₂CH₂NHCH₃, —OCH₂CH₂N(CH₃)₂, —OCH₂CH₂OCF₃, —OCH₂CH₂OCHF₂, —OCH₂CH₂OCFH₂, —OCH₂C₆H₅, —OCH₂CH₂C₆H₅, —OCH₂C₆H₅-o-Cl, —OCH₂C₆H₅-m-Cl and —OCH₂C₆H₅-p-Cl.

In some embodiments, R₅ is selected from the group consisting of —OCH₃, —OCH₂CH₃, —OCH(CH₃)₂, hydroxyl, —OCH₂CH₂N(CH₃)₂, —OCH₂C₆H₅, —OCH₂CH₂C₆H₅ and —OCH₂C₆H₅-p-Cl.

In some embodiments, R₅ is —OCH₃.

Some embodiments pertains to compounds wherein R₆ is selected from the group consisting of H, C₁₋₆ alkoxy, carbo-C₁₋₆-alkoxy, carboxamide, carboxy, cyano, halogen and hydroxyl.

In some embodiments, R₆ is H.

Some embodiments pertain to compounds wherein R₆ₐ, R_{6b}, and R_{6c} are each independently selected from the group consisting of H, C₁₋₆ alkoxy, C₁₋₆ alkyl, amino, C₁₋₆ alkylamino, C₂₋₈ dialkylamino, cyano, halogen, C₁₋₆ haloalkoxy, C₁₋₆ haloalkyl, hydroxyl, and nitro.

Some embodiments pertain to compounds wherein R₆ₐ, R_{6b}, and R_{6c} are each independently selected from the group consisting of H, —OCH₃, —CH₃, —N(CH₃)₂, cyano, —F, —Cl, —Br, —OCF₃, hydroxyl, and nitro.

Some embodiments pertain to compounds wherein R₆ₐ, R_{6b}, and R_{6c} are each independently selected from the group consisting of H, C₁₋₆ alkoxy, carbo-C₁₋₆-alkoxy, carboxamide, carboxy, cyano, halogen and hydroxyl.

Some embodiments pertain to compounds wherein R₆ₐ, R_{6b}, and R_{6c} are all H.

Some embodiments pertain to compounds wherein R₅ is C₁₋₆ alkoxy and R₆ₐ, R_{6b}, and R_{6c} are all H.

In some embodiments, R₅ is —OCH₃.

Some embodiments pertain to compounds represented by Formula (Ip) as shown below:

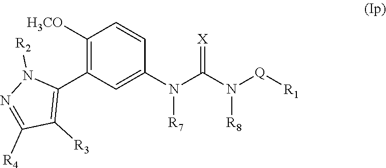

wherein each variable in Formula (Ip) has the same meaning as described herein, supra and infra. In some embodiments, compounds have Formula (Ip) and Q is a bond.

Some embodiments pertain to compounds represented by Formula (Iq) as shown below:

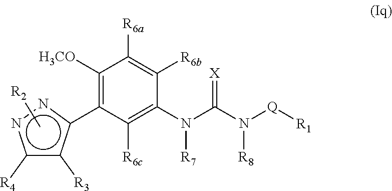

wherein each variable in Formula (Iq) has the same meaning as described herein, supra and infra. In some embodiments, compounds have Formula (Iq) and Q is a bond.

Some embodiments pertain to compounds wherein R₇ is H or C₁₋₈ alkyl.

In some embodiments, R₇ is selected from the group consisting of H, —CH₃, —CH₂CH₃, —CH(CH₃)₂, —CH₂CH₂CH₃, —CH₂CH(CH₃)₂ and —CH₂CH₂CH₂CH₃.

In some embodiments, R₇ is H.

Some embodiments pertain to compounds wherein R₈ is H or C₁₋₈ alkyl.

In some embodiments, R₈ is selected from the group consisting of H, —CH₃, —CH₂CH₃, —CH(CH₃)₂, —CH₂CH₂CH₃, —CH₂CH(CH₃)₂ and —CH₂CH₂CH₂CH₃.

In some embodiments, R₈ is H.

Some embodiments pertain to compounds wherein both R₇ and R₈ are H.

Some embodiments pertain to compounds represented by Formula (Ir) as shown below:

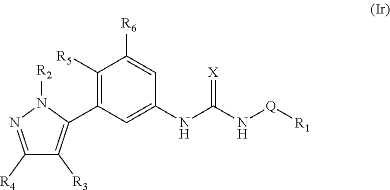

wherein each variable in Formula (Ir) has the same meaning as described herein, supra and infra.

Some embodiments pertain to compounds represented by Formula (Is) as shown below:

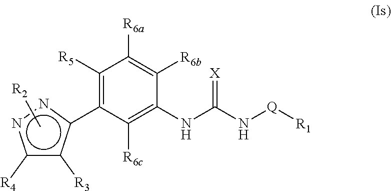

wherein each variable in Formula (Is) has the same meaning as described herein, supra and infra.

Some embodiments pertain to compounds wherein X is O (i.e., oxygen).

Some embodiments pertain to compounds wherein X is S (i.e., sulfur).

Some embodiments pertain to compounds wherein Q is C₁₋₃ alkylene optionally substituted with C₁₋₃ alkyl, C₁₋₃ haloalkyl, halogen and oxo.

Some embodiments pertain to compounds wherein Q is a C₁₋₃ alkylene optionally substituted with oxo. As used herein, oxo refers to a double bonded oxygen. In some embodiments, Q is —C(O)— (i.e., a carbonyl).

In some embodiments, Q is —CH₂—.

Some embodiments pertain to compounds wherein Q is a bond.

Some embodiments can be represented by Formula (It) as shown below:

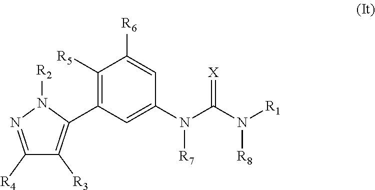

wherein each variable in Formula (It) has the same meaning as described herein, supra and infra.

Some embodiments can be represented by Formula (Iu) as shown below:

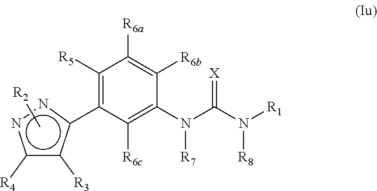

wherein each variable in Formula (Iu) has the same meaning as described herein, supra and infra.

In some embodiments, R₁ is phenyl and can be represented by Formula (Iv) as shown below:

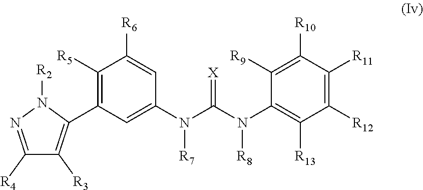

wherein each variable in Formula (Iv) has the same meaning as described herein, supra and infra. In some embodiments, R₇ and R₈ are both H. In some embodiments, X is O (i.e., oxygen).

In some embodiments, R₁ is phenyl and can be represented by Formula (Iw) as shown below:

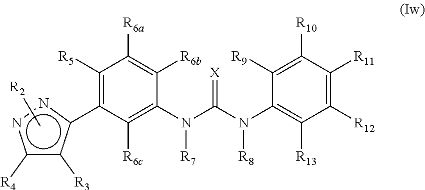

wherein each variable in Formula (Iw) has the same meaning as described herein, supra and infra. In some embodiments, R₇ and R₈ are both H. In some embodiments, X is O (i.e., oxygen).

Some embodiments pertain to compounds of Formula (IIa):

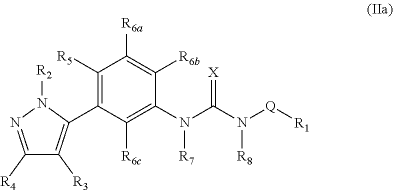

wherein:

R₁ is phenyl or naphthyl optionally substituted with R₉, R₁₀, R₁₁, R₁₂, R₁₃, R₁₄, and R₁₅ each selected independently from the group consisting of C₁₋₆ acyl, C₁₋₆ alkoxy, C₁₋₆ alkyl, amino, C₁₋₆ alkylamino, C₂₋₈ dialkylamino, C₁₋₆ alkylimino, cyano, halogen, C₁₋₆ haloalkoxy, C₁₋₆ haloalkyl, heterocyclic, hydroxyl, nitro, and phenyl, or two adjacent R₉, R₁₀, R₁₁, R₁₂, R₁₃, R₁₄, and R₁₅ together with the atoms to which they are attached form a C₅₋₇ cycloalkyl group or heterocyclic group each optionally substituted with F; and wherein said C₁₋₆ alkyl, C₁₋₆ alkylimino, and heterocyclic are each optionally substituted with 1 to 5 substituents selected independently from the group consisting of C₁₋₆ alkyl, amino, C₁₋₆ alkylamino, C₂₋₈ dialkylamino, and hydroxyl;

R₂ is C₁₋₆ alkyl;

R₃ is H or halogen;

R₄ is selected from the group consisting of H, C₁₋₆ alkyl and C₁₋₆ haloalkyl;

R₅ is selected from the group consisting of C₁₋₆ alkoxy, C₁₋₆ haloalkoxy, and hydroxyl, wherein said C₁₋₆ alkoxy group can be optionally substituted with 1 to 5 substituents selected independently from the group consisting of amino, C₂₋₈ dialkylamino, carboxy, and phenyl, and wherein said amino and phenyl are each optionally substituted with 1 to 5 further substituents selected from the group consisting of halogen and carbo-C₁₋₆-alkoxy;

R₆ₐ, R_{6b}, and R_{6c} are each independently selected from the group consisting of H, C₁₋₆ alkoxy, C₁₋₆ alkyl, amino, C₁₋₆ alkylamino, C₂₋₈ dialkylamino, cyano, halogen, C₁₋₆ haloalkoxy, C₁₋₆ haloalkyl, hydroxyl, and nitro

R₇ and R₈ are both H;

X is O; and

Q is a bond.

Some embodiments pertain to compounds of Formula (IIa):

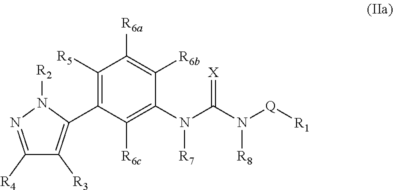

wherein:

R₁ is phenyl or naphthyl optionally substituted with R₉, R₁₀, R₁₁, R₁₂, R₁₃, R₁₄, and R₁₅ each selected independently from the group consisting of —C(O)CH₃, —OCH₃, —CH₃, —CH(CH₃)₂, —CH(OH)CH₃, —N(CH₃)₂, (2-dimethylamino-ethyl)-methyl-amino, (3-dimethylamino-propyl)-methyl-amino, —C(═NOH)CH₃, cyano, —F, —Cl, —Br, —OCF₃, —CF₃, 4-methyl-piperazin-1-yl, morpholin-4-yl, 4-methyl-piperidin-1-yl, hydroxyl, nitro, and phenyl;

R₂ is —CH₃ or —CH(CH₃)₂;

R₃ is H, F, Cl, or Br;

R₄ is —H, or —CF₃;

R₅ is selected from the group consisting of —OCH₃, —OCH₂CH₃, —OCH(CH₃)₂, —OCF₃, hydroxyl, benzyloxy, 4-chloro-benzyloxy, phenethyloxy, 2-dimethylamino-ethoxy, 3-dimethylamino-propoxy, carboxymethoxy, and 2-tert-butoxycarbonylamino-ethoxy;

R₆ₐ, R_{6b}, and R_{6c} are each independently selected from the group consisting of H, —OCH₃, —CH₃, —N(CH₃)₂, cyano, —F, —Cl, —Br, —OCF₃, hydroxyl, and nitro;

R₇ and R₈ are both H;

X is O; and

Q is a bond.

Some embodiments pertain to compounds of Formula (IIa):

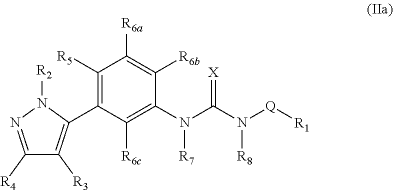

wherein:

R₁ is phenyl optionally substituted with R₉, R₁₀, R₁₁, R₁₂, and R₁₃ each selected independently from the group consisting of —C(O)CH₃, —OCH₃, —CH₃, —CH(CH₃)₂, —CH(OH)CH₃, —N(CH₃)₂, (2-dimethylamino-ethyl)-methyl-amino, (3-dimethylamino-propyl)-methyl-amino, —C(═NOH)CH₃, cyano, —F, —Cl, —Br, —OCF₃, —CF₃, 4-methyl-piperazin-1-yl, morpholin-4-yl, 4-methyl-piperidin-1-yl, hydroxyl, nitro, and phenyl;

R₂ is —CH₃ or —CH(CH₃)₂;

R₃ is —H, —F, —Cl, or —Br;

R₄ is —H, or —CF₃;

R₅ is selected from the group consisting of —OCH₃, —OCH₂CH₃, —OCH(CH₃)₂, —OCF₃, hydroxyl, benzyloxy, 4-chloro-benzyloxy, phenethyloxy, 2-dimethylamino-ethoxy, 3-dimethylamino-propoxy, carboxymethoxy, and 2-tert-butoxycarbonylamino-ethoxy;

R₆ₐ, R_{6b}, and R_{6c} are each independently selected from the group consisting of —H, —OCH₃, —CH₃, —N(CH₃)₂, cyano, F, Cl, Br, —OCF₃, hydroxyl, and nitro;

R₇ and R₈ are both H;

X is O; and

Q is a bond.

Some embodiments pertain to compounds of Formula (IIa):

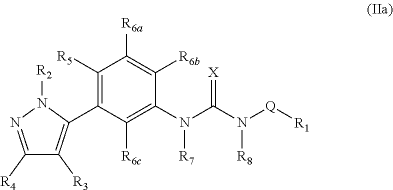

wherein:

R₁ is phenyl optionally substituted with R₉, R₁₀, R₁₁, R₁₂, and R₁₃ each selected independently from the group consisting of —C(O)CH₃, —OCH₃, —CH₃, —CH(CH₃)₂, —N(CH₃)₂, cyano, —F, —Cl, —Br, —OCF₃, —CF₃, hydroxyl, and nitro;

R₂ is —CH₃;

R₃ is —H, —F, —Cl, or —Br;

R₄ is —H;

R₅ is selected from the group consisting of —OCH₃, —OCH₂CH₃, —OCH(CH₃)₂, —OCF₃, hydroxyl, benzyloxy, 4-chloro-benzyloxy, phenethyloxy, 2-dimethylamino-ethoxy, 3-dimethylamino-propoxy, carboxymethoxy, and 2-tert-butoxycarbonylamino-ethoxy;

R₆ₐ, R_{6b}, and R_{6c} are each —H;

R₇ and R₈ are both —H;

X is O; and

Q is a bond.

Some embodiments include compounds illustrated in TABLE A as shown below:

TABLE A

| Cmpd# | Structure | Chemical Name |
|---|---|---|
| 1 | 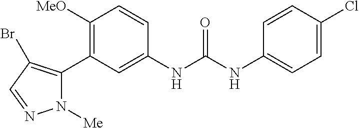 [SMILES: COc1ccc(NC(=O)Nc2ccc(Cl)cc2)cc1-c1c(Br)cnn1C] | 1-[3-(4-Bromo-2-methyl-2H- pyrazol-3-yl)-4-methoxy- phenyl]-3-(4-chloro-phenyl)-urea |
| 2 | 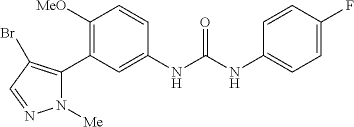 [SMILES: COc1ccc(NC(=O)Nc2ccc(F)cc2)cc1-c1c(Br)cnn1C] | 1-[3-(4-Bromo-2-methyl-2H- pyrazol-3-yl)-4-methoxy- phenyl]-3-(4-fluoro-phenyl)-urea |
| 3 | 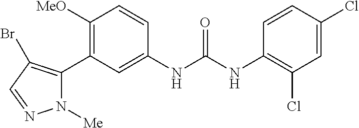 [SMILES: COc1ccc(NC(=O)Nc2ccc(Cl)cc2Cl)cc1-c1c(Br)cnn1C] | 1-[3-(4-Bromo-2-methyl-2H- pyrazol-3-yl)-4-methoxy- phenyl]-3-(2,4-dichloro-phenyl)- urea |
| 4 | 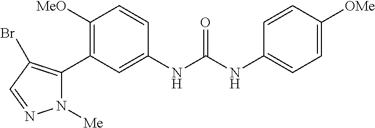 [SMILES: COc1ccc(NC(=O)Nc2ccc(OC)c(-c3c(Br)cnn3C)c2)cc1] | 1-[3-(4-Bromo-2-methyl-2H- pyrazol-3-yl)-4-methoxy- phenyl]-3-(4-methoxy-phenyl)- urea |
| 5 | 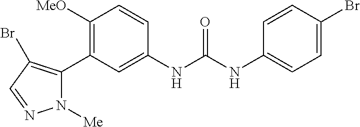 [SMILES: COc1ccc(NC(=O)Nc2ccc(Br)cc2)cc1-c1c(Br)cnn1C] | 1-[3-(4-Bromo-2-methyl-2H- pyrazol-3-yl)-4-methoxy- phenyl]-3-(4-bromo-phenyl)- urea |
| 6 | 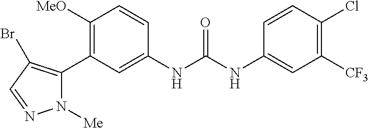 [SMILES: COc1ccc(NC(=O)Nc2ccc(Cl)c(C(F)(F)F)c2)cc1-c1c(Br)cnn1C] | 1-[3-(4-Bromo-2-methyl-2H- pyrazol-3-yl)-4-methoxy- phenyl]-3-(4-chloro-3- trifluoromethyl-phenyl)-urea |
| 7 | 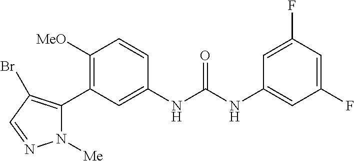 [SMILES: COc1ccc(NC(=O)Nc2cc(F)cc(F)c2)cc1-c1c(Br)cnn1C] | 1-[3-(4-Bromo-2-methyl-2H- pyrazol-3-yl)-4-methoxy- phenyl]-3-(3,5-difluoro-phenyl)- urea |
| 8 | 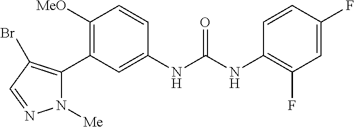 [SMILES: COc1ccc(NC(=O)Nc2ccc(F)cc2F)cc1-c1c(Br)cnn1C] | 1-[3-(4-Bromo-2-methyl-2H- pyrazol-3-yl)-4-methoxy- phenyl]-3-(2,4-difluoro-phenyl)- urea (nelotanserin) |
| 9 | 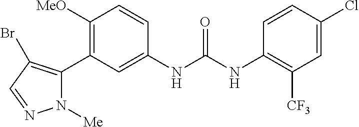 [SMILES: COc1ccc(NC(=O)Nc2ccc(Cl)cc2C(F)(F)F)cc1-c1c(Br)cnn1C] | 1-[3-(4-Bromo-2-methyl-2H- pyrazol-3-yl)-4-methoxy- phenyl]-3-(4-chloro-2- trifluoromethyl-phenyl)-urea |
| 10 | 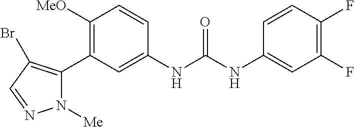 [SMILES: COc1ccc(NC(=O)Nc2ccc(F)c(F)c2)cc1-c1c(Br)cnn1C] | 1-[3-(4-Bromo-2-methyl-2H- pyrazol-3-yl)-4-methoxy- phenyl]-3-(3,4-difluoro-phenyl)- urea |
| 11 | 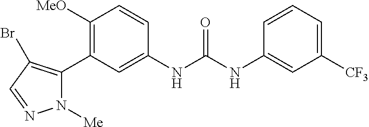 [SMILES: COc1ccc(NC(=O)Nc2cccc(C(F)(F)F)c2)cc1-c1c(Br)cnn1C] | 1-[3-(4-Bromo-2-methyl-2H- pyrazol-3-yl)-4-methoxy- phenyl]-3-(3-trifluoromethyl- phenyl)-urea |
| 12 | 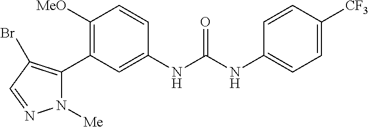 [SMILES: COc1ccc(NC(=O)Nc2ccc(C(F)(F)F)cc2)cc1-c1c(Br)cnn1C] | 1-[3-(4-Bromo-2-methyl-2H- pyrazol-3-yl)-4-methoxy- phenyl]-3-(4-trifluoromethyl- phenyl)-urea |
| 13 | 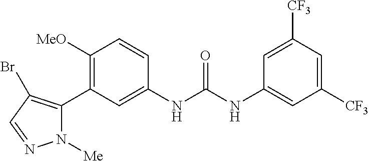 [SMILES: COc1ccc(NC(=O)Nc2cc(C(F)(F)F)cc(C(F)(F)F)c2)cc1-c1c(Br)cnn1C] | 1-(3,5-Bis-trifluoromethyl- phenyl)-3-[3-(4-bromo-2- methyl-2H-pyrazol-3-yl)-4- methoxy-phenyl]-urea |
| 14 | 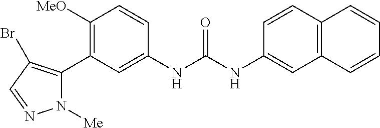 [SMILES: COc1ccc(NC(=O)Nc2ccc3ccccc3c2)cc1-c1c(Br)cnn1C] | 1-[3-(4-Bromo-2-methyl-2H- pyrazol-3-yl)-4-methoxy- phenyl]-3-naphthalen-2-yl-urea |
| 15 | 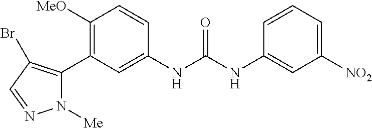 [SMILES: COc1ccc(NC(=O)Nc2cccc([N+](=O)[O-])c2)cc1-c1c(Br)cnn1C] | 1-[3-(4-Bromo-2-methyl-2H- pyrazol-3-yl)-4-methoxy- phenyl]-3-(3-nitro-phenyl)-urea |
| 16 | 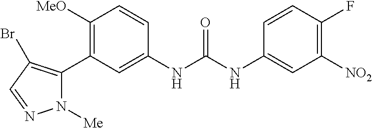 [SMILES: COc1ccc(NC(=O)Nc2ccc(F)c([N+](=O)[O-])c2)cc1-c1c(Br)cnn1C] | 1-[3-(4-Bromo-2-methyl-2H- pyrazol-3-yl)-4-methoxy- phenyl]-3-(4-fluoro-3-nitro- phenyl)-urea |
| 17 | 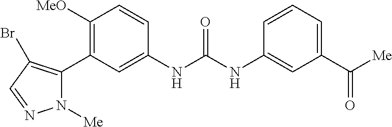 [SMILES: COc1ccc(NC(=O)Nc2cccc(C(C)=O)c2)cc1-c1c(Br)cnn1C] | 1-(3-Acetyl-phenyl)-3-[3-(4- bromo-2-methyl-2H-pyrazol-3- yl)-4-methoxy-phenyl]-urea |
| 18 | 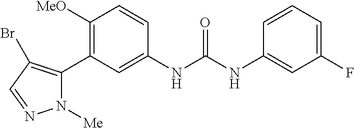 [SMILES: COc1ccc(NC(=O)Nc2cccc(F)c2)cc1-c1c(Br)cnn1C] | 1-[3-(4-Bromo-2-methyl-2H- pyrazol-3-yl)-4-methoxy- phenyl]-3-(3-fluoro-phenyl)-urea |
| 19 | 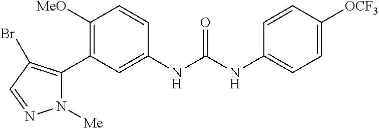 [SMILES: COc1ccc(NC(=O)Nc2ccc(OC(F)(F)F)cc2)cc1-c1c(Br)cnn1C] | 1-[3-(4-Bromo-2-methyl-2H- pyrazol-3-yl)-4-methoxy- phenyl]-3-(4-trifluoromethoxy- phenyl)-urea |
| 20 | 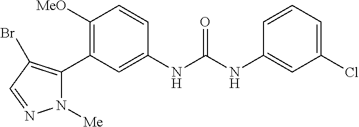 [SMILES: COc1ccc(NC(=O)Nc2cccc(Cl)c2)cc1-c1c(Br)cnn1C] | 1-[3-(4-Bromo-2-methyl-2H- pyrazol-3-yl)-4-methoxy- phenyl]-3-(3-chloro-phenyl)-urea |
| 21 | 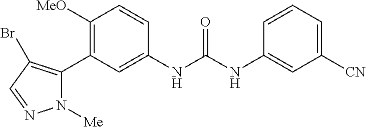 [SMILES: COc1ccc(NC(=O)Nc2cccc(C#N)c2)cc1-c1c(Br)cnn1C] | 1-[3-(4-Bromo-2-methyl-2H- pyrazol-3-yl)-4-methoxy- phenyl]-3-(3-cyano-phenyl)-urea |
| 22 | 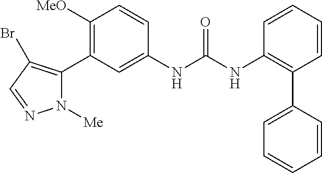 [SMILES: COc1ccc(NC(=O)Nc2ccccc2-c2ccccc2)cc1-c1c(Br)cnn1C] | 1-Biphenyl-2-yl-3-[3-(4-bromo- 2-methyl-2H-pyrazol-3-yl)-4- methoxy-phenyl]-urea |
| 23 | 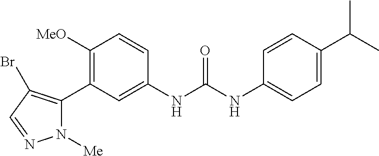 [SMILES: COc1ccc(NC(=O)Nc2ccc(C(C)C)cc2)cc1-c1c(Br)cnn1C] | 1-[3-(4-Bromo-2-methyl-2H- pyrazol-3-yl)-4-methoxy- phenyl]-3-(4-i sopropyl-phenyl)- urea |
| 24 | 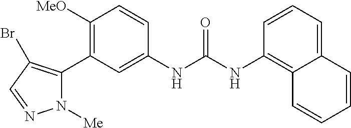 [SMILES: COc1ccc(NC(=O)Nc2cccc3ccccc23)cc1-c1c(Br)cnn1C] | 1-[3-(4-Bromo-2-methyl-2H- pyrazol-3-yl)-4-methoxy- phenyl]-3-naphthalen-1-yl-urea |
| 25 | 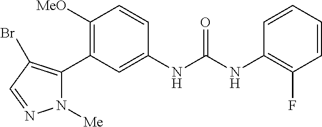 [SMILES: COc1ccc(NC(=O)Nc2ccccc2F)cc1-c1c(Br)cnn1C] | 1-[3-(4-Bromo-2-methyl-2H- pyrazol-3-yl)-4-methoxy- phenyl]-3-(2-fluoro-phenyl)-urea |
| 26 | 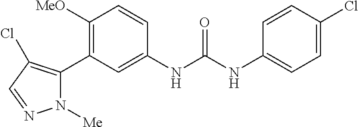 [SMILES: COc1ccc(NC(=O)Nc2ccc(Cl)cc2)cc1-c1c(Cl)cnn1C] | 1-[3-(4-Chloro-2-methyl-2H- pyrazol-3-yl)-4-methoxy- phenyl]-3-(4-chloro-phenyl)-urea |
| 27 | 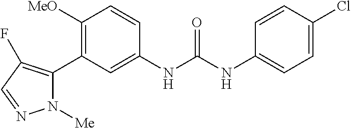 [SMILES: COc1ccc(NC(=O)Nc2ccc(Cl)cc2)cc1-c1c(F)cnn1C] | 1-(4-Chloro-phenyl)-3-[3-(4- fluoro-2-methyl-2H-pyrazol-3- yl)-4-methoxy-phenyl]-urea |
| 28 | 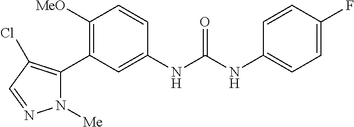 [SMILES: COc1ccc(NC(=O)Nc2ccc(F)cc2)cc1-c1c(Cl)cnn1C] | 1-[3-(4-Chloro-2-methyl-2H- pyrazol-3-yl)-4-methoxy- phenyl]-3-(4-fluoro-phenyl)-urea |
| 29 | 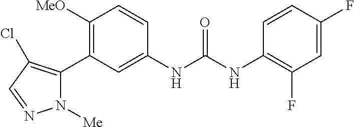 [SMILES: COc1ccc(NC(=O)Nc2ccc(F)cc2F)cc1-c1c(Cl)cnn1C] | 1-[3-(4-Chloro-2-methyl-2H- pyrazol-3-yl)-4-methoxy- phenyl]-3-(2,4-difluoro-phenyl)- urea |
| 30 | 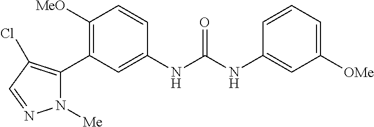 [SMILES: COc1cccc(NC(=O)Nc2ccc(OC)c(-c3c(Cl)cnn3C)c2)c1] | 1-[3-(4-Chloro-2-methyl-2H- pyrazol-3-yl)-4-methoxy- phenyl]-3-(3-methoxy-phenyl)- urea |
| 31 | 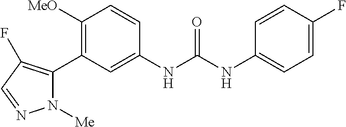 [SMILES: COc1ccc(NC(=O)Nc2ccc(F)cc2)cc1-c1c(F)cnn1C] | 1-[3-(4-Fluoro-2-methyl-2H- pyrazol-3-yl)-4-methoxy- phenyl]-3-(4-fluoro-phenyl)-urea |
| 32 | 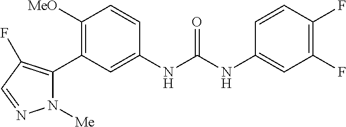 [SMILES: COc1ccc(NC(=O)Nc2ccc(F)c(F)c2)cc1-c1c(F)cnn1C] | 1-(3,4-Difluoro-phenyl)-3-[3-(4- fluoro-2-methyl-2H-pyrazol-3- yl)-4-methoxy-phenyl]-urea |
| 33 | 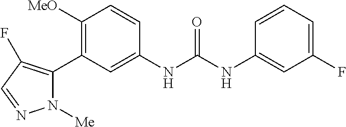 [SMILES: COc1ccc(NC(=O)Nc2cccc(F)c2)cc1-c1c(F)cnn1C] | 1-[3-(4-Fluoro-2-methyl-2H- pyrazol-3-yl)-4-methoxy- phenyl]-3-(3-fluoro-phenyl)-urea |
| 34 | 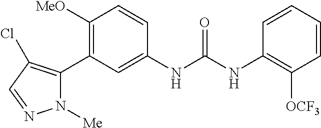 [SMILES: COc1ccc(NC(=O)Nc2ccccc2OC(F)(F)F)cc1-c1c(Cl)cnn1C] | 1-[3-(4-Chloro-2-methyl-2H- pyrazol-3-yl)-4-methoxy- phenyl]-3-(2-trifluoromethoxy- phenyl)-urea |
| 35 | 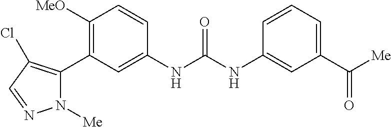 [SMILES: COc1ccc(NC(=O)Nc2cccc(C(C)=O)c2)cc1-c1c(Cl)cnn1C] | 1-(3-Acetyl-phenyl)-3-[3-(4- chloro-2-methyl-2H-pyrazol-3- yl)-4-methoxy-phenyl]-urea |
| 36 | 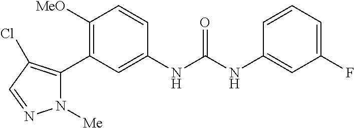 [SMILES: COc1ccc(NC(=O)Nc2cccc(F)c2)cc1-c1c(Cl)cnn1C] | 1-[3-(4-Chloro-2-methyl-2H- pyrazol-3-yl)-4-methoxy- phenyl]-3-(3-fluoro-phenyl)-urea |
| 37 | 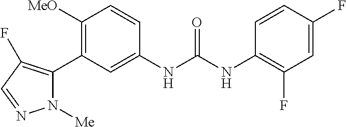 [SMILES: COc1ccc(NC(=O)Nc2ccc(F)cc2F)cc1-c1c(F)cnn1C] | 1-(2,4-Difluoro-phenyl)-3-[3-(4- fluoro-2-methyl-2H-pyrazol-3- yl)-4-methoxy-phenyl]-urea |
| 38 | 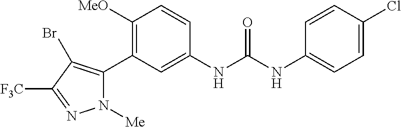 [SMILES: COc1ccc(NC(=O)Nc2ccc(Cl)cc2)cc1-c1c(Br)c(C(F)(F)F)nn1C] | 1-[3-(4-Bromo-2-methyl-5- trifluoromethyl-2H-pyrazol-3- yl)-4-methoxy-phenyl]-3-(4- chloro-phenyl)-urea |
| 39 | 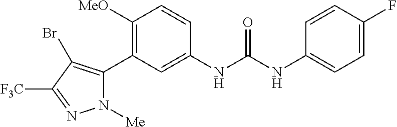 [SMILES: COc1ccc(NC(=O)Nc2ccc(F)cc2)cc1-c1c(Br)c(C(F)(F)F)nn1C] | 1-[3-(4-Bromo-2-methyl-5- trifluoromethyl-2H-pyrazol-3- yl)-4-methoxy-phenyl]-3-(4- fluoro-phenyl)-urea |
| 40 | 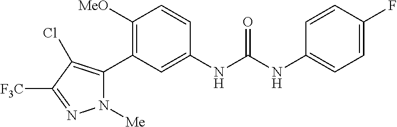 [SMILES: COc1ccc(NC(=O)Nc2ccc(F)cc2)cc1-c1c(Cl)c(C(F)(F)F)nn1C] | 1-[3-(4-Chloro-2-methyl-5- trifluoromethyl-2H-pyrazol-3- yl)-4-methoxy-phenyl]-3-(4- fluoro-phenyl)-urea |
| 41 | 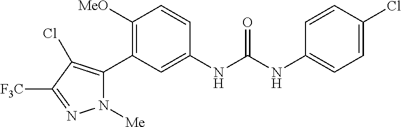 [SMILES: COc1ccc(NC(=O)Nc2ccc(Cl)cc2)cc1-c1c(Cl)c(C(F)(F)F)nn1C] | 1-[3-(4-Chloro-2-methyl-5- trifluoromethyl-2H-pyrazol-3- yl)-4-methoxy-phenyl]-3-(4- chloro-phenyl)-urea |
| 42 | 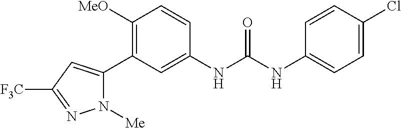 [SMILES: COc1ccc(NC(=O)Nc2ccc(Cl)cc2)cc1-c1cc(C(F)(F)F)nn1C] | 1-(4-Chloro-phenyl)-3-[4- methoxy-3-(2-methyl-5- trifluoromethyl-2H-pyrazol-3- yl)-phenyl]-urea |
| 43 | 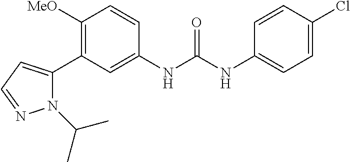 [SMILES: COc1ccc(NC(=O)Nc2ccc(Cl)cc2)cc1-c1ccnn1C(C)C] | 1-(4-Chloro-phenyl)-3-[3-(2- isopropyl-2H-pyrazol-3-yl)-4- methoxy-phenyl]-urea |
| 44 | 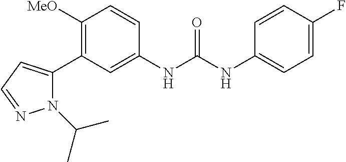 [SMILES: COc1ccc(NC(=O)Nc2ccc(F)cc2)cc1-c1ccnn1C(C)C] | 1-(4-Fluoro-phenyl)-3-[3-(2- isopropyl-2H-pyrazol-3-yl)-4- methoxy-phenyl]-urea |
| 45 | 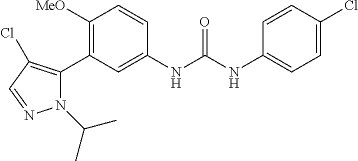 [SMILES: COc1ccc(NC(=O)Nc2ccc(Cl)cc2)cc1-c1c(Cl)cnn1C(C)C] | 1-[3-(4-Chloro-2-isopropyl-2H- pyrazol-3-yl)-4-methoxy- phenyl]-3-(4-chloro-phenyl)-urea |
| 46 | 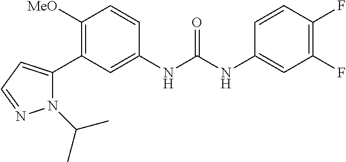 [SMILES: COc1ccc(NC(=O)Nc2ccc(F)c(F)c2)cc1-c1ccnn1C(C)C] | 1-(3,4-Difluoro-phenyl)-3-[3-(2- isopropyl-2H-pyrazol-3-yl)-4- methoxy-phenyl]-urea |
| 47 | 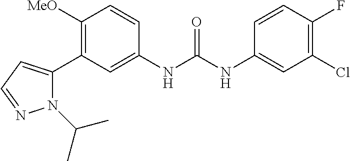 [SMILES: COc1ccc(NC(=O)Nc2ccc(F)c(Cl)c2)cc1-c1ccnn1C(C)C] | 1-(3-Chloro-4-fluoro-phenyl)-3- [3-(2-isopropyl-2H-pyrazol-3- yl)-4-methoxy-phenyl]-urea |
| 48 | 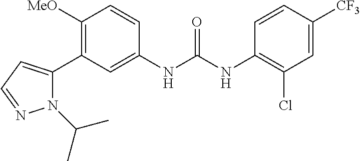 [SMILES: COc1ccc(NC(=O)Nc2ccc(C(F)(F)F)cc2Cl)cc1-c1ccnn1C(C)C] | 1-(2-Chloro-4-trifluoromethyl- phenyl)-3-[3-(2-isopropyl-2H- pyrazol-3-yl)-4-methoxy- phenyl]-urea |
| 49 | 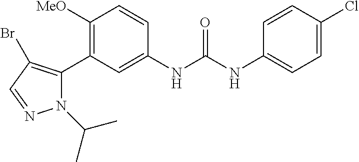 [SMILES: COc1ccc(NC(=O)Nc2ccc(Cl)cc2)cc1-c1c(Br)cnn1C(C)C] | 1-[3-(4-Bromo-2-isopropyl-2H- pyrazol-3-yl)-4-methoxy- phenyl]-3-(4-chloro-phenyl)-urea |
| 50 | 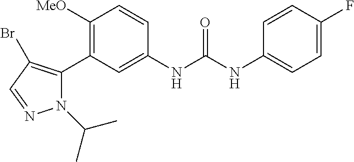 [SMILES: COc1ccc(NC(=O)Nc2ccc(F)cc2)cc1-c1c(Br)cnn1C(C)C] | 1-[3-(4-Bromo-2-isopropyl-2H- pyrazol-3-yl)-4-methoxy- phenyl]-3-(4-fluoro-phenyl)-urea |
| 51 | 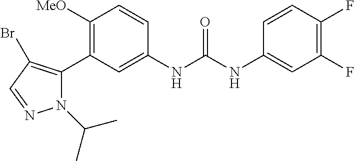 [SMILES: COc1ccc(NC(=O)Nc2ccc(F)c(F)c2)cc1-c1c(Br)cnn1C(C)C] | 1-[3-(4-Bromo-2-isopropyl-2H- pyrazol-3-yl)-4-methoxy- phenyl]-3-(3,4-difluoro-phenyl)- urea |
| 52 | 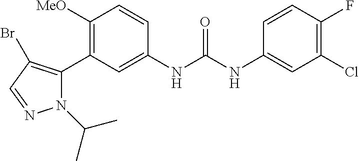 [SMILES: COc1ccc(NC(=O)Nc2ccc(F)c(Cl)c2)cc1-c1c(Br)cnn1C(C)C] | 1-[3-(4-Bromo-2-isopropyl-2H- pyrazol-3-yl)-4-methoxy- phenyl]-3-(3-chloro-4-fluoro- phenyl)-urea |
| 53 | 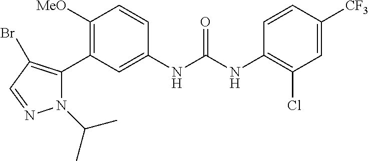 [SMILES: COc1ccc(NC(=O)Nc2ccc(C(F)(F)F)cc2Cl)cc1-c1c(Br)cnn1C(C)C] | 1-[3-(4-Bromo-2-isopropyl-2H- pyrazol-3-yl)-4-methoxy- phenyl]-3-(2-Chloro-4- trifluoromethyl-phenyl)-urea |
| 54 | 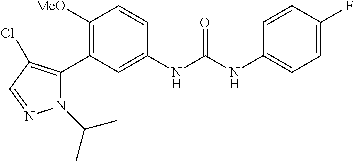 [SMILES: COc1ccc(NC(=O)Nc2ccc(F)cc2)cc1-c1c(Cl)cnn1C(C)C] | 1-[3-(4-Chloro-2-isopropyl-2H- pyrazol-3-yl)-4-methoxy- phenyl]-3-(4-fluoro-phenyl)-urea |
| 55 | 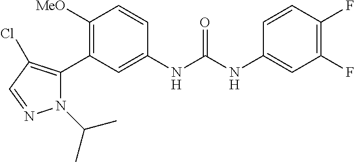 [SMILES: COc1ccc(NC(=O)Nc2ccc(F)c(F)c2)cc1-c1c(Cl)cnn1C(C)C] | 1-[3-(4-Chloro-2-isopropyl-2H- pyrazol-3-yl)-4-methoxy- phenyl]-3-(3,4-difluoro-phenyl)- urea |
| 56 | 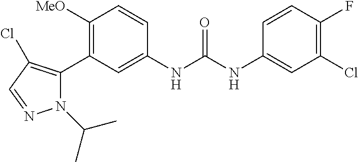 [SMILES: COc1ccc(NC(=O)Nc2ccc(F)c(Cl)c2)cc1-c1c(Cl)cnn1C(C)C] | 1-(3-Chloro-4-fluoro-phenyl)-3- [3-(4-Chloro-2-isopropyl-2H- pyrazol-3-yl)-4-methoxy- phenyl]-urea |
| 57 | 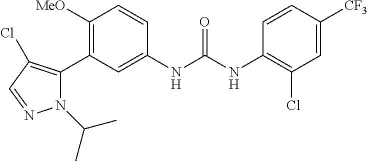 [SMILES: COc1ccc(NC(=O)Nc2ccc(C(F)(F)F)cc2Cl)cc1-c1c(Cl)cnn1C(C)C] | 1-[3-(4-Chloro-2-isopropyl-2H- pyrazol-3-yl)-4-methoxy- phenyl]-3-(2-Chloro-4- trifluoromethyl-phenyl)-urea |
| 58 | 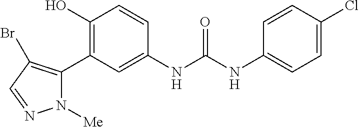 [SMILES: Cn1ncc(Br)c1-c1cc(NC(=O)Nc2ccc(Cl)cc2)ccc1O] | 1-[3-(4-Bromo-2-methyl-2H- pyrazol-3-yl)-4-hydroxy- phenyl]-3-(4-chloro-phenyl)-urea |
| 59 | 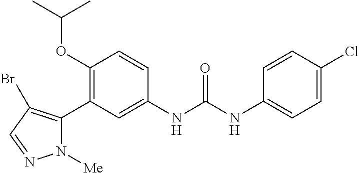 [SMILES: CC(C)Oc1ccc(NC(=O)Nc2ccc(Cl)cc2)cc1-c1c(Br)cnn1C] | 1-[3-(4-Bromo-2-methyl-2H- pyrazol-3-yl)-4-isopropoxy- phenyl]-3-(4-chloro-phenyl)-urea |
| 60 | 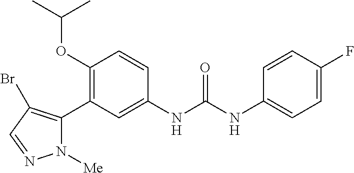 [SMILES: CC(C)Oc1ccc(NC(=O)Nc2ccc(F)cc2)cc1-c1c(Br)cnn1C] | 1-[3-(4-Bromo-2-methyl-2H- pyrazol-3-yl)-4-isopropoxy- phenyl]-3-(4-fluoro-phenyl)-urea |
| 61 | 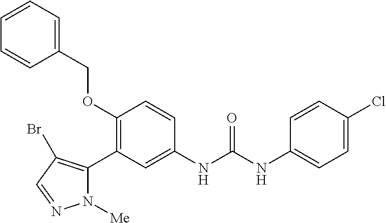 [SMILES: Cn1ncc(Br)c1-c1cc(NC(=O)Nc2ccc(Cl)cc2)ccc1OCc1ccccc1] | 1-[4-Benzyloxy-3-(4-bromo-2- methyl-2H-pyrazol-3-yl)- phenyl]-3-(4-chloro-phenyl)-urea |
| 62 | 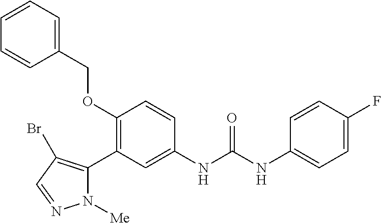 [SMILES: Cn1ncc(Br)c1-c1cc(NC(=O)Nc2ccc(F)cc2)ccc1OCc1ccccc1] | 1-[4-Benzyloxy-3-(4-bromo-2- methyl-2H-pyrazol-3-yl)- phenyl]-3-(4-fluoro-phenyl)-urea |
| 63 | 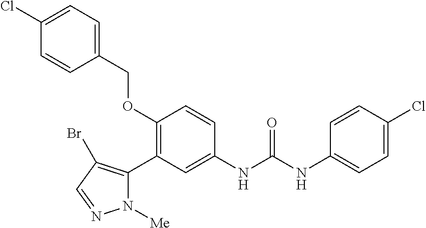 [SMILES: Cn1ncc(Br)c1-c1cc(NC(=O)Nc2ccc(Cl)cc2)ccc1OCc1ccc(Cl)cc1] | 1-[3-(4-Bromo-2-methyl-2H- pyrazol-3-yl)-4-(4-chloro- benzyloxy)-phenyl]-3-(4-chloro- phenyl)-urea |
| 64 | 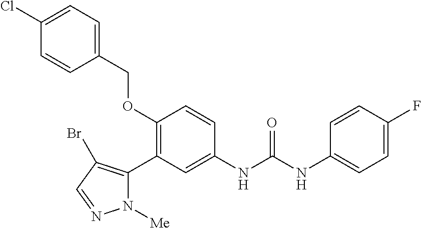 [SMILES: Cn1ncc(Br)c1-c1cc(NC(=O)Nc2ccc(F)cc2)ccc1OCc1ccc(Cl)cc1] | 1-[3-(4-Bromo-2-methyl-2H- pyrazol-3-yl)-4-(4-chloro- benzyloxy)-phenyl]-3-(4-fluoro- phenyl)-urea |
| 65 | 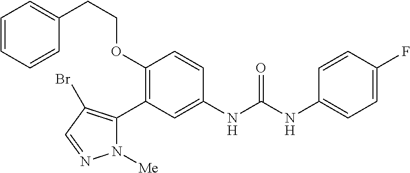 [SMILES: Cn1ncc(Br)c1-c1cc(NC(=O)Nc2ccc(F)cc2)ccc1OCCc1ccccc1] | 1-[3-(4-Bromo-2-methyl-2H- pyrazol-3-yl)-4-phenethyloxy- phenyl]-3-(4-fluoro-phenyl)-urea |
| 66 | 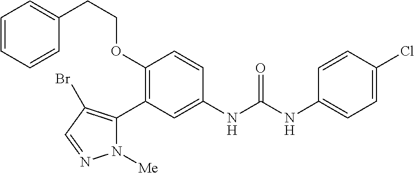 [SMILES: Cn1ncc(Br)c1-c1cc(NC(=O)Nc2ccc(Cl)cc2)ccc1OCCc1ccccc1] | 1-[3-(4-Bromo-2-methyl-2H- pyrazol-3-yl)-4-phenethyloxy- phenyl]-3-(4-chloro-phenyl)-urea |
| 67 | 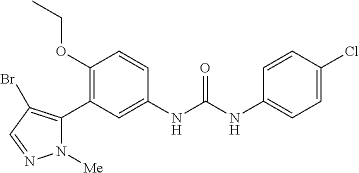 [SMILES: CCOc1ccc(NC(=O)Nc2ccc(Cl)cc2)cc1-c1c(Br)cnn1C] | 1-[3-(4-Bromo-2-methyl-2H- pyrazol-3-yl)-4-ethoxy-phenyl]- 3-(4-chloro-phenyl)-urea |
| 68 | 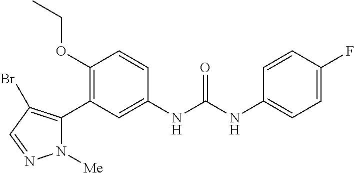 [SMILES: CCOc1ccc(NC(=O)Nc2ccc(F)cc2)cc1-c1c(Br)cnn1C] | 1-[3-(4-Bromo-2-methyl-2H- pyrazol-3-yl)-4-ethoxy-phenyl]- 3-(4-fluoro-phenyl)-urea |
| 69 | 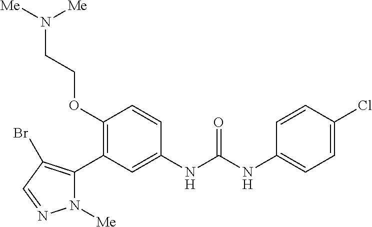 [SMILES: CN(C)CCOc1ccc(NC(=O)Nc2ccc(Cl)cc2)cc1-c1c(Br)cnn1C] | 1-[3-(4-Bromo-2-methyl-2H- pyrazol-3-yl)-4-(2- dimethylamino-ethoxy)-phenyl]- 3-(4-chloro-phenyl)-urea |
| 70 | 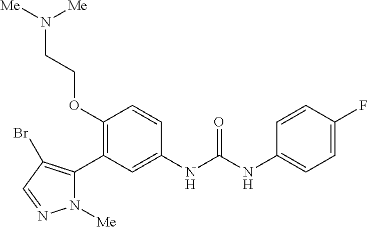 [SMILES: CN(C)CCOc1ccc(NC(=O)Nc2ccc(F)cc2)cc1-c1c(Br)cnn1C] | 1-[3-(4-Bromo-2-methyl-2H- pyrazol-3-yl)-4-(2- dimethylamino-ethoxy)-phenyl]- 3-(4-fluoro-phenyl)-urea |
| 71 | 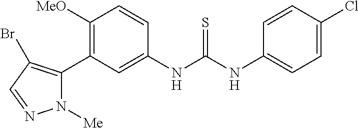 [SMILES: COc1ccc(NC(=S)Nc2ccc(Cl)cc2)cc1-c1c(Br)cnn1C] | 1-[3-(4-Bromo-2-methyl-2H- pyrazol-3-yl)-4-methoxy- phenyl]-3-(4-chloro-phenyl)- thiourea |
| 72 | 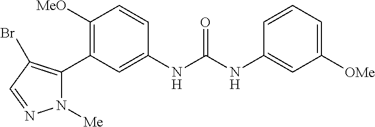 [SMILES: COc1cccc(NC(=O)Nc2ccc(OC)c(-c3c(Br)cnn3C)c2)c1] | 1-[3-(4-Bromo-2-methyl-2H- pyrazol-3-yl)-4-methoxy- phenyl]-3-(3-methoxy-phenyl)- urea |
| 73 | 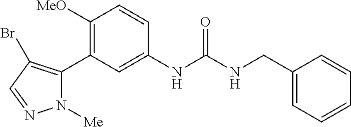 [SMILES: COc1ccc(NC(=O)NCc2ccccc2)cc1-c1c(Br)cnn1C] | 1-Benzoyl-3-[3-(4-bromo-2- methyl-2H-pyrazol-3-yl)-4- methoxy-phenyl]-urea |
| 74 | 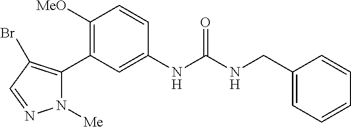 [SMILES: COc1ccc(NC(=O)NCc2ccccc2)cc1-c1c(Br)cnn1C] | 1-Benzyl-3-[3-(4-bromo-2- methyl-2H-pyrazol-3-yl)-4- methoxy-phenyl]-urea |
| 75 | 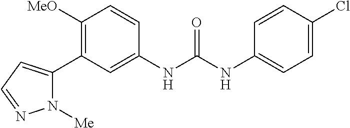 [SMILES: COc1ccc(NC(=O)Nc2ccc(Cl)cc2)cc1-c1ccnn1C] | 1-(4-Chloro-phenyl)-3-[4- methoxy-3-(2-methyl-2H- pyrazol-3-yl)-phenyl]-urea |
| 76 | 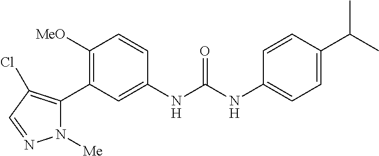 [SMILES: COc1ccc(NC(=O)Nc2ccc(C(C)C)cc2)cc1-c1c(Cl)cnn1C] | 1-[3-(4-Chloro-2-methyl-2H- pyrazol-3-yl)-4-methoxy- phenyl]-3-(4-isopropyl-phenyl)- urea |
| 77 | 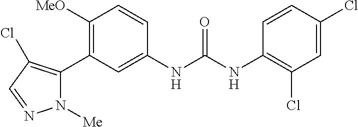 [SMILES: COc1ccc(NC(=O)Nc2ccc(Cl)cc2Cl)cc1-c1c(Cl)cnn1C] | 1-[3-(4-Chloro-2-methyl-2H- pyrazol-3-yl)-4-methoxy- phenyl]-3-(2,4-dichloro-phenyl)- urea |
| 78 | 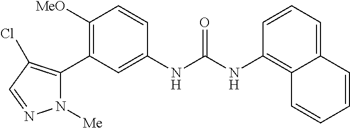 [SMILES: COc1ccc(NC(=O)Nc2cccc3ccccc23)cc1-c1c(Cl)cnn1C] | 1-[3-(4-Chloro-2-methyl-2H- pyrazol-3-yl)-4-methoxy- phenyl]-3-naphthalen-1-yl-urea |
| 79 | 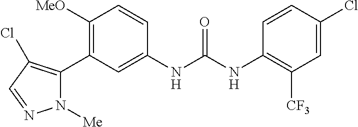 [SMILES: COc1ccc(NC(=O)Nc2ccc(Cl)cc2C(F)(F)F)cc1-c1c(Cl)cnn1C] | 1-[3-(4-Chloro-2-methyl-2H- pyrazol-3-yl)-4-methoxy- phenyl]-3-(4-chloro-2- trifluoromethyl-phenyl)-urea |
| 80 | 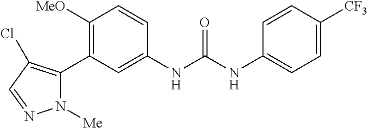 [SMILES: COc1ccc(NC(=O)Nc2ccc(C(F)(F)F)cc2)cc1-c1c(Cl)cnn1C] | 1-[3-(4-Chloro-2-methyl-2H- pyrazol-3-yl)-4-methoxy- phenyl]-3-(4-trifluoromethyl- phenyl)-urea |
| 81 | 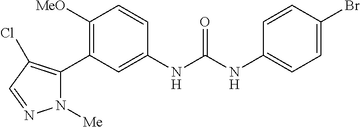 [SMILES: COc1ccc(NC(=O)Nc2ccc(Br)cc2)cc1-c1c(Cl)cnn1C] | 1-(4-Bromo-phenyl)-3-[3-(4- chloro-2-methyl-2H-pyrazol-3- yl)-4-methoxy-phenyl]-urea |
| 82 | 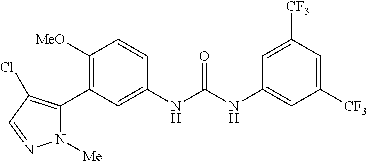 [SMILES: COc1ccc(NC(=O)Nc2cc(C(F)(F)F)cc(C(F)(F)F)c2)cc1-c1c(Cl)cnn1C] | 1-(3,5-Bis-trifluoromethyl- phenyl)-3-[3-(4-chloro-2-methyl- 2H-pyrazol-3-yl)-4-methoxy- phenyl]-urea |
| 83 | 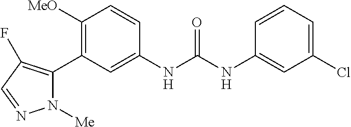 [SMILES: COc1ccc(NC(=O)Nc2cccc(Cl)c2)cc1-c1c(F)cnn1C] | 1-(3-Chloro-phenyl)-3-[3-(4- fluoro-2-methyl-2H-pyrazol-3- yl)-4-methoxy-phenyl]-urea |
| 84 | 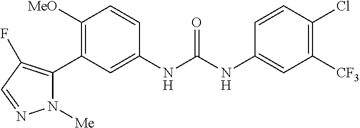 [SMILES: COc1ccc(NC(=O)Nc2ccc(Cl)c(C(F)(F)F)c2)cc1-c1c(F)cnn1C] | 1-(4-Chloro-3-trifluoromethyl- phenyl)-3-[3-(4-fluoro-2-methyl- 2H-pyrazol-3-yl)-4-methoxy- phenyl]-urea |
| 85 | 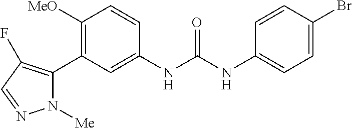 [SMILES: COc1ccc(NC(=O)Nc2ccc(Br)cc2)cc1-c1c(F)cnn1C] | 1-(4-Bromo-phenyl)-3-[3-(4- fluoro-2-methyl-2H-pyrazol-3- yl)-4-methoxy-phenyl]-urea |
| 86 | 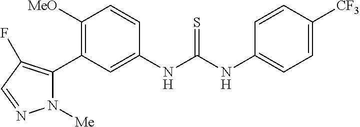 [SMILES: COc1ccc(NC(=S)Nc2ccc(C(F)(F)F)cc2)cc1-c1c(F)cnn1C] | 1-[3-(4-Fluoro-2-methyl-2H- pyrazol-3-yl)-4-methoxy- phenyl]-3-(4-trifluoromethyl- phenyl)-thiourea |
| 87 | 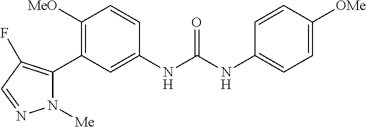 [SMILES: COc1ccc(NC(=O)Nc2ccc(OC)c(-c3c(F)cnn3C)c2)cc1] | 1-[3-(4-Fluoro-2-methyl-2H- pyrazol-3-yl)-4-methoxy- phenyl]-3-(4-methoxy-phenyl)- urea |
| 88 | 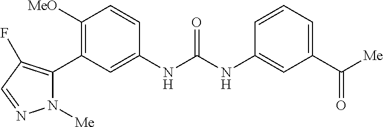 [SMILES: COc1ccc(NC(=O)Nc2cccc(C(C)=O)c2)cc1-c1c(F)cnn1C] | 1-(3-Acetyl-phenyl)-3-[3-(4- fluoro-2-methyl-2H-pyrazol-3- yl)-4-methoxy-phenyl]-urea |
| 89 | 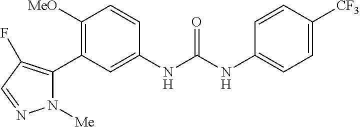 [SMILES: COc1ccc(NC(=O)Nc2ccc(C(F)(F)F)cc2)cc1-c1c(F)cnn1C] | 1-[3-(4-Fluoro-2-methyl-2H- pyrazol-3-yl)-4-methoxy- phenyl]-3-(4-trifluoromethyl- phenyl)-urea |
| 90 | 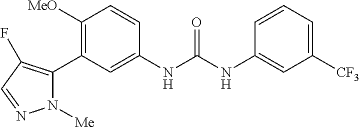 [SMILES: COc1ccc(NC(=O)Nc2cccc(C(F)(F)F)c2)cc1-c1c(F)cnn1C] | 1-[3-(4-Fluoro-2-methyl-2H- pyrazol-3-yl)-4-methoxy- phenyl]-3-(3-trifluoromethyl- phenyl)-urea |
| 91 | 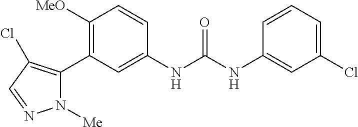 [SMILES: COc1ccc(NC(=O)Nc2cccc(Cl)c2)cc1-c1c(Cl)cnn1C] | 1-[3-(4-Chloro-2-methyl-2H- pyrazol-3-yl)-4-methoxy- phenyl]-3-(3-chloro-phenyl)-urea |
| 92 | 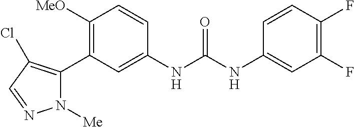 [SMILES: COc1ccc(NC(=O)Nc2ccc(F)c(F)c2)cc1-c1c(Cl)cnn1C] | 1-[3-(4-Chloro-2-methyl-2H- pyrazol-3-yl)-4-methoxy- phenyl]-3-(3,4-difluoro-phenyl)- urea |
| 93 | 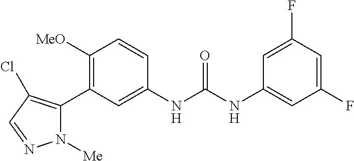 [SMILES: COc1ccc(NC(=O)Nc2cc(F)cc(F)c2)cc1-c1c(Cl)cnn1C] | 1-[3-(4-Chloro-2-methyl-2H- pyrazol-3-yl)-4-methoxy- phenyl]-3-(3,5-difluoro-phenyl)- urea |
| 94 | 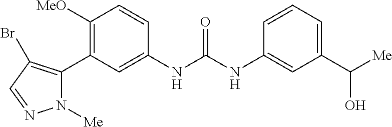 [SMILES: COc1ccc(NC(=O)Nc2cccc(C(C)O)c2)cc1-c1c(Br)cnn1C] | 1-[3-(4-Bromo-2-methyl-2H- pyrazol-3-yl)-4-methoxy- phenyl]-3-[3-(1-hydroxy-ethyl)- phenyl]-urea |
| 95 | 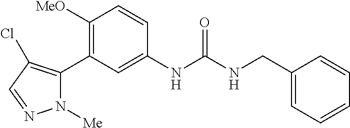 [SMILES: COc1ccc(NC(=O)NCc2ccccc2)cc1-c1c(Cl)cnn1C] | 1-Benzoyl-3-[3-(4-chloro-2- methyl-2H-pyrazol-3-yl)-4- methoxy-phenyl]-urea |
| 96 | 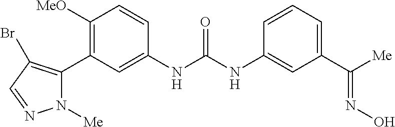 [SMILES: COc1ccc(NC(=O)Nc2cccc(/C(C)=N/O)c2)cc1-c1c(Br)cnn1C] | 1-[3-(4-Bromo-2-methyl-2H- pyrazol-3-yl)-4-methoxy- phenyl]-3-[3-(1-hydroxylmino- ethyl)-phenyl]-urea |
| 97 | 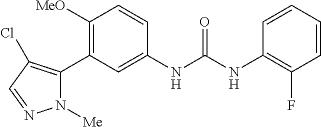 [SMILES: COc1ccc(NC(=O)Nc2ccccc2F)cc1-c1c(Cl)cnn1C] | 1-[3-(4-Chloro-2-methyl-2H- pyrazol-3-yl)-4-methoxy- phenyl]-3-(2-fluoro-phenyl)-urea |
| 98 | 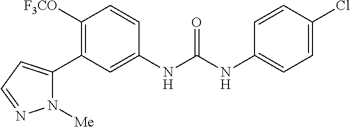 [SMILES: Cn1nccc1-c1cc(NC(=O)Nc2ccc(Cl)cc2)ccc1OC(F)(F)F] | 1-(4-Chloro-phenyl)-3-[3-(2- methyl-2H-pyrazol-3-yl)-4- trifluoromethoxy-phenyl]-urea |
| 99 | 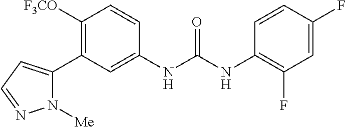 [SMILES: Cn1nccc1-c1cc(NC(=O)Nc2ccc(F)cc2F)ccc1OC(F)(F)F] | 1-(2,4-Difluoro-phenyl)-3-[3-(2- methyl-2H-pyrazol-3-yl)-4- trifluoromethoxy-phenyl]-urea |
| 100 | 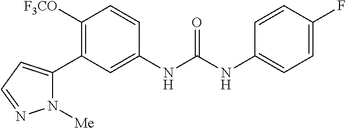 [SMILES: Cn1nccc1-c1cc(NC(=O)Nc2ccc(F)cc2)ccc1OC(F)(F)F] | 1-(4-Fluoro-phenyl)-3-[3-(2- methyl-2H-pyrazol-3-yl)-4- trifluoromethoxy-phenyl]-urea |
| 101 | 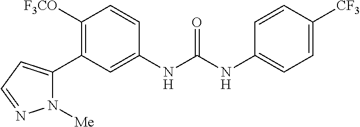 [SMILES: Cn1nccc1-c1cc(NC(=O)Nc2ccc(C(F)(F)F)cc2)ccc1OC(F)(F)F] | 1-[3-(2-Methyl-2H-pyrazol-3- yl)-4-trifluoromethoxy-phenyl]- 3-(4-trifluoromethyl-phenyl)- urea |
| 102 | 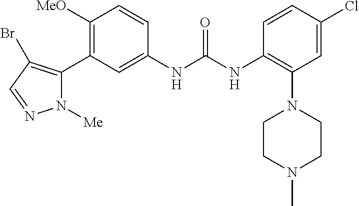 [SMILES: COc1ccc(NC(=O)Nc2ccc(Cl)cc2N2CCN(C)CC2)cc1-c1c(Br)cnn1C] | 1-[3-(4-Bromo-2-methyl-2H- pyrazol-3-yl)-4-methoxy- phenyl]-3-[4-chloro-2-(4-methyl- piperazin-1-yl)-phenyl]-urea |
| 103 | 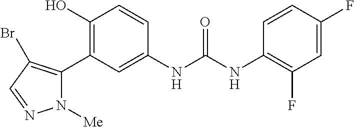 [SMILES: Cn1ncc(Br)c1-c1cc(NC(=O)Nc2ccc(F)cc2F)ccc1O] | 1-[3-(4-Bromo-2-methyl-2H- pyrazol-3-yl)-4-hydroxy- phenyl]-3-(2,4-difluoro-phenyl)- urea |
| 104 | 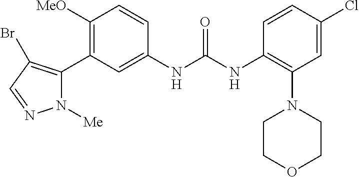 [SMILES: COc1ccc(NC(=O)Nc2ccc(Cl)cc2N2CCOCC2)cc1-c1c(Br)cnn1C] | 1-[3-(4-Bromo-2-methyl-2H- pyrazol-3-yl)-4-methoxy- phenyl]-3-(4-chloro-2- morpholin-4-yl-phenyl)-urea |
| 105 | 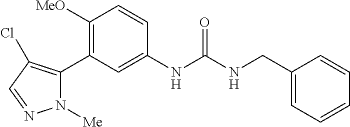 [SMILES: COc1ccc(NC(=O)NCc2ccccc2)cc1-c1c(Cl)cnn1C] | 1-Benzyl-3-[3-(4-chloro-2- methyl-2H-pyrazol-3-yl)-4- methoxy-phenyl]-urea |
| 106 | 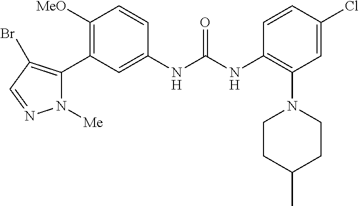 [SMILES: COc1ccc(NC(=O)Nc2ccc(Cl)cc2N2CCC(C)CC2)cc1-c1c(Br)cnn1C] | 1-[3-(4-Bromo-2-methyl-2H- pyrazol-3-yl)-4-methoxy- phenyl]-3-[4-chloro-2-(4-methyl- piperidin-1-yl)-phenyl]-urea |
| 107 | 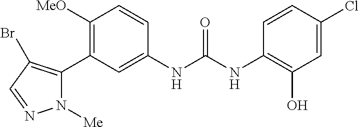 [SMILES: COc1ccc(NC(=O)Nc2ccc(Cl)cc2O)cc1-c1c(Br)cnn1C] | 1-[3-(4-Bromo-2-methyl-2H- pyrazol-3-yl)-4-methoxy- phenyl]-3-(4-chloro-2-hydroxy- phenyl)-urea |
| 108 | 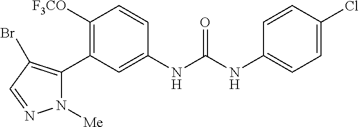 [SMILES: Cn1ncc(Br)c1-c1cc(NC(=O)Nc2ccc(Cl)cc2)ccc1OC(F)(F)F] | 1-[3-(4-Bromo-2-methyl-2H- pyrazol-3-yl)-4- trifluoromethoxy-phenyl]-3-(4- chloro-phenyl)-urea |
| 109 | 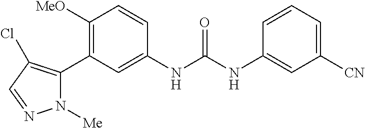 [SMILES: COc1ccc(NC(=O)Nc2cccc(C#N)c2)cc1-c1c(Cl)cnn1C] | 1-[3-(4-Chloro-2-methyl-2H- pyrazol-3-yl)-4-methoxy- phenyl]-3-(3-cyano-phenyl)-urea |
| 110 | 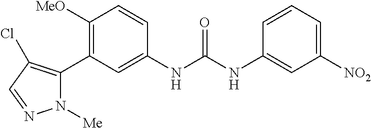 [SMILES: COc1ccc(NC(=O)Nc2cccc([N+](=O)[O-])c2)cc1-c1c(Cl)cnn1C] | 1-[3-(4-Chloro-2-methyl-2H- pyrazol-3-yl)-4-methoxy- phenyl]-3-(3-nitro-phenyl)-urea |
| 111 | 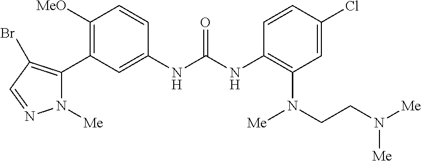 [SMILES: COc1ccc(NC(=O)Nc2ccc(Cl)cc2N(C)CCN(C)C)cc1-c1c(Br)cnn1C] | 1-[3-(4-Bromo-2-methyl-2H- pyrazol-3-yl)-4-methoxy- phenyl]-3-{4-chloro-2-[(2- dimethylamino-ethyl)-methyl- amino]-phenyl}-urea |
| 112 | 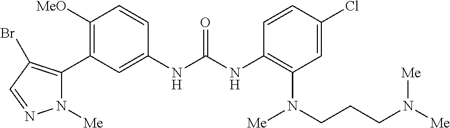 [SMILES: COc1ccc(NC(=O)Nc2ccc(Cl)cc2N(C)CCCN(C)C)cc1-c1c(Br)cnn1C] | 1-[3-(4-Bromo-2-methyl-2H- pyrazol-3-yl)-4-methoxy- phenyl]-3-{4-chloro-2-[(3- dimethylamino-propyl)-methyl- amino]-phenyl}-urea |
| 113 | 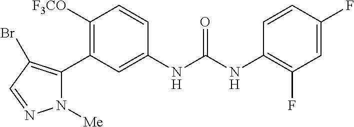 [SMILES: Cn1ncc(Br)c1-c1cc(NC(=O)Nc2ccc(F)cc2F)ccc1OC(F)(F)F] | 1-[3-(4-Bromo-2-methyl-2H- pyrazol-3-yl)-4- trifluoromethoxy-phenyl]-3-(2,4- difluoro-phenyl)-urea |
| 114 | 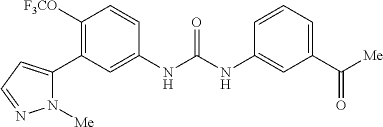 [SMILES: CC(=O)c1cccc(NC(=O)Nc2ccc(OC(F)(F)F)c(-c3ccnn3C)c2)c1] | 1-(3-Acetyl-phenyl)-3-[3-(2- methyl-2H-pyrazol-3-yl)-4- trifluoromethoxy-phenyl]-urea |
| 115 | 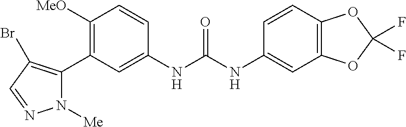 [SMILES: COc1ccc(NC(=O)Nc2ccc3c(c2)OC(F)(F)O3)cc1-c1c(Br)cnn1C] | 1-[3-(4-Bromo-2-methyl-2H- pyrazol-3-yl)-4-methoxy- phenyl]-3-(2,2-difluoro- benzo[1,3]dioxol-5-yl)-urea |
| 116 | 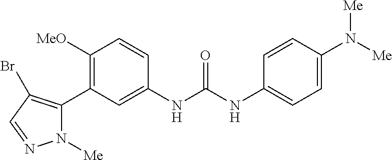 [SMILES: COc1ccc(NC(=O)Nc2ccc(N(C)C)cc2)cc1-c1c(Br)cnn1C] | 1-[3-(4-Bromo-2-methyl-2H- pyrazol-3-yl)-4-methoxy- phenyl]-3-(4-dimethylamino- phenyl)-urea |
| 117 | 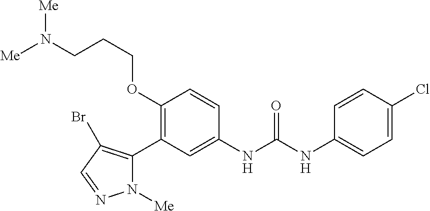 [SMILES: CN(C)CCCOc1ccc(NC(=O)Nc2ccc(Cl)cc2)cc1-c1c(Br)cnn1C] | 1-[3-(4-Bromo-2-methyl-2H- pyrazol-3-yl)-4-(3- dimethylamino-propoxy)- phenyl]-3-(4-chloro-phenyl)-urea |
| 118 | 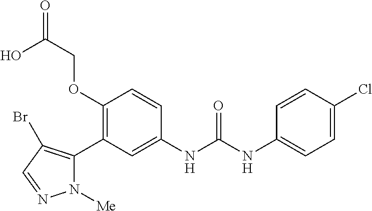 [SMILES: Cn1ncc(Br)c1-c1cc(NC(=O)Nc2ccc(Cl)cc2)ccc1OCC(=O)O] | {2-(4-Bromo-2-methyl-2H- pyrazol-3-yl)-4-[3-(4-chloro- phenyl)-ureido]-phenoxy}-acetic acid |
| 119 | 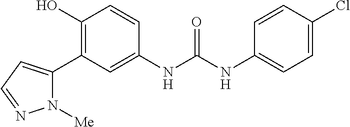 [SMILES: Cn1nccc1-c1cc(NC(=O)Nc2ccc(Cl)cc2)ccc1O] | 1-(4-Chloro-phenyl)-3-[4- hydroxy-3-(2-methyl-2H- pyrazol-3-yl)-phenyl]-urea |
| 120 | 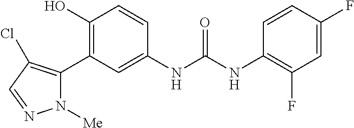 [SMILES: Cn1ncc(Cl)c1-c1cc(NC(=O)Nc2ccc(F)cc2F)ccc1O] | 1-[3-(4-Chloro-2-methyl-2H- pyrazol-3-yl)-4-hydroxy- phenyl]-3-(2,4-difluoro-phenyl)- urea |
| 121 | 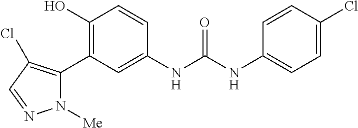 [SMILES: Cn1ncc(Cl)c1-c1cc(NC(=O)Nc2ccc(Cl)cc2)ccc1O] | 1-[3-(4-Chloro-2-methyl-2H- pyrazol-3-yl)-4-hydroxy- phenyl]-3-(4-chloro-phenyl)-urea |
| 122 | 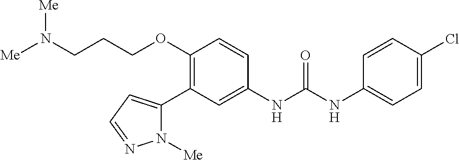 [SMILES: CN(C)CCCOc1ccc(NC(=O)Nc2ccc(Cl)cc2)cc1-c1ccnn1C] | 1-(4-Chloro-phenyl)-3-[4-(3- dimethylamino-propoxy)-3-(2- methyl-2H-pyrazol-3-yl)- phenyl]-urea |
| 123 | 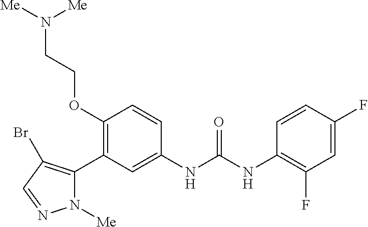 [SMILES: CN(C)CCOc1ccc(NC(=O)Nc2ccc(F)cc2F)cc1-c1c(Br)cnn1C] | 1-[3-(4-Bromo-2-methyl-2H- pyrazol-3-yl)-4-(2- dimethylamino-ethoxy)-phenyl]- 3-(2,4-difluoro-phenyl)-urea |
| 124 | 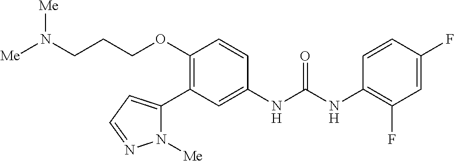 [SMILES: CN(C)CCCOc1ccc(NC(=O)Nc2ccc(F)cc2F)cc1-c1ccnn1C] | 1-(2,4-Difluoro-phenyl)-3-[4-(3- dimethylamino-propoxy)-3-(2- methyl-2H-pyrazol-3-yl)- phenyl]-urea |
| 125 | 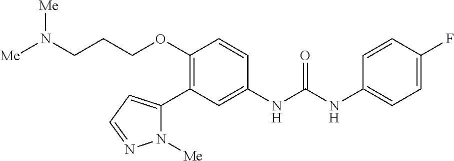 [SMILES: CN(C)CCCOc1ccc(NC(=O)Nc2ccc(F)cc2)cc1-c1ccnn1C] | 1-[4-(3-Dimethylamino- propoxy)-3-(2-methyl-2H- pyrazol-3-yl)-phenyl]-3-(4- fluoro-phenyl)-urea |
| 126 | 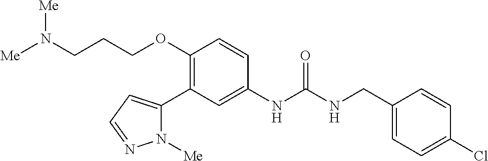 [SMILES: CN(C)CCCOc1ccc(NC(=O)NCc2ccc(Cl)cc2)cc1-c1ccnn1C] | 1-(4-Chloro-benzyl)-3-[4-(3- dimethylamino-propoxy)-3-(2- methyl-2H-pyrazol-3-yl)- phenyl]-urea |
| 127 | 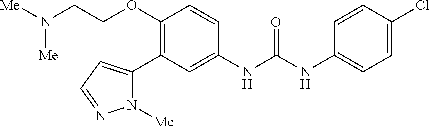 [SMILES: CN(C)CCOc1ccc(NC(=O)Nc2ccc(Cl)cc2)cc1-c1ccnn1C] | 1-(4-Chloro-phenyl)-3-[4-(2- dimethylamino-ethoxy)-3-(2- methyl-2H-pyrazol-3-yl)- phenyl]-urea |
| 128 | 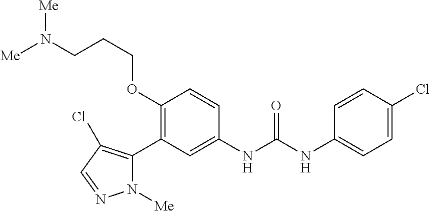 [SMILES: CN(C)CCCOc1ccc(NC(=O)Nc2ccc(Cl)cc2)cc1-c1c(Cl)cnn1C] | 1-[3-(4-Chloro-2-methyl-2H- pyrazol-3-yl)-4-(3- dimethylamino-propoxy)- phenyl]-3-(4-chloro-phenyl)-urea |
| 129 | 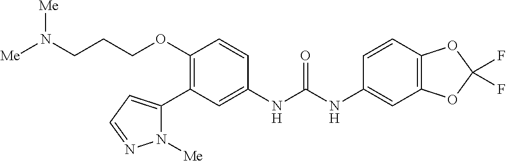 [SMILES: CN(C)CCCOc1ccc(NC(=O)Nc2ccc3c(c2)OC(F)(F)O3)cc1-c1ccnn1C] | 1-(2,2-Difluoro- benzo[1,3]dioxol-5-yl)-3-[4-(3- dimethylamino-propoxy)-3-(2- methyl-2H-pyrazol-3-yl)- phenyl]-urea |
| 130 | 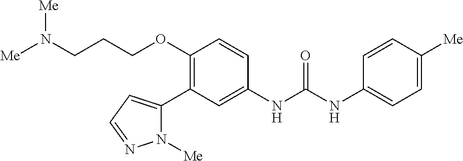 [SMILES: Cc1ccc(NC(=O)Nc2ccc(OCCCN(C)C)c(-c3ccnn3C)c2)cc1] | 1-[4-(3-Dimethylamino- propoxy)-3-(2-methyl-2H- pyrazol-3-yl)-phenyl]-3-p-tolyl- urea |
| 131 | 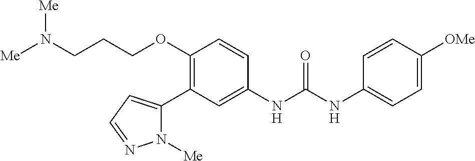 [SMILES: COc1ccc(NC(=O)Nc2ccc(OCCCN(C)C)c(-c3ccnn3C)c2)cc1] | 1-[4-(3-Dimethylamino- propoxy)-3-(2-methyl-2H- pyrazol-3-yl)-phenyl]-3-(4- methoxy-phenyl)-urea |
| 132 | 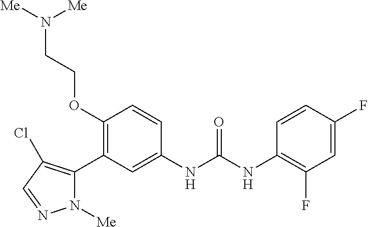 [SMILES: CN(C)CCOc1ccc(NC(=O)Nc2ccc(F)cc2F)cc1-c1c(Cl)cnn1C] | 1-[3-(4-Chloro-2-methyl-2H- pyrazol-3-yl)-4-(2- dimethylamino-ethoxy)-phenyl]- 3-(2,4-difluoro-phenyl)-urea |
| 133 | 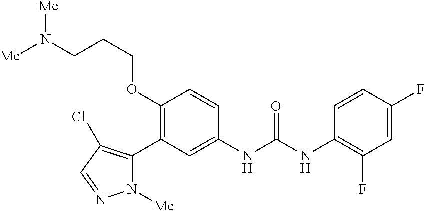 [SMILES: CN(C)CCCOc1ccc(NC(=O)Nc2ccc(F)cc2F)cc1-c1c(Cl)cnn1C] | 1-[3-(4-Chloro-2-methyl-2H- pyrazol-3-yl)-4-(3- dimethylamino-propoxy)- phenyl]-3-(2,4-difluoro-phenyl)- urea |
| 134 | 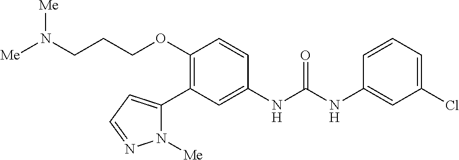 [SMILES: CN(C)CCCOc1ccc(NC(=O)Nc2cccc(Cl)c2)cc1-c1ccnn1C] | 1-(3-Chloro-phenyl)-3-[4-(3- dimethylamino-propoxy)-3-(2- methyl-2H-pyrazol-3-yl)- phenyl]-urea |
| 135 | 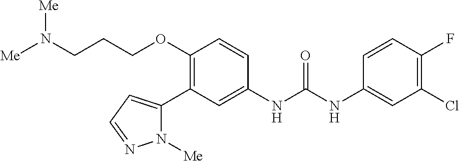 [SMILES: CN(C)CCCOc1ccc(NC(=O)Nc2ccc(F)c(Cl)c2)cc1-c1ccnn1C] | 1-(3-Chloro-4-fluoro-phenyl)-3- [4-(3-dimethylamino-propoxy)- 3-(2-methyl-2H-pyrazol-3-yl)- phenyl]-urea |
| 136 | 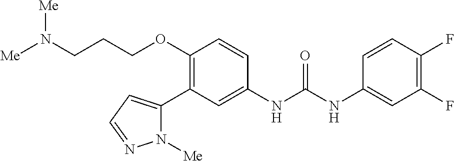 [SMILES: CN(C)CCCOc1ccc(NC(=O)Nc2ccc(F)c(F)c2)cc1-c1ccnn1C] | 1-(3,4-Difluoro-phenyl)-3-[4-(3- dimethylamino-propoxy)-3-(2- methyl-2H-pyrazol-3-yl)- phenyl]-urea |
| 137 | 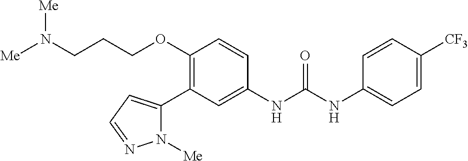 [SMILES: CN(C)CCCOc1ccc(NC(=O)Nc2ccc(C(F)(F)F)cc2)cc1-c1ccnn1C] | 1-[4-(3-Dimethylamino- propoxy)-3-(2-methyl-2H- pyrazol-3-yl)-phenyl]-3-(4- trifluoromethyl-phenyl)-urea |
| 138 | 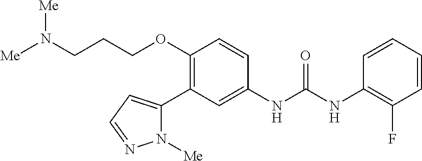 [SMILES: CN(C)CCCOc1ccc(NC(=O)Nc2ccccc2F)cc1-c1ccnn1C] | 1-[4-(3-Dimethylamino- propoxy)-3-(2-methyl-2H- pyrazol-3-yl)-phenyl]-3-(2- fluoro-phenyl)-urea |
| 139 | 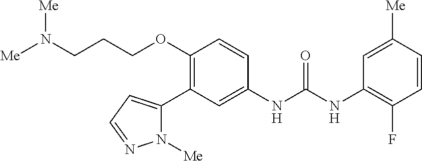 [SMILES: Cc1ccc(F)c(NC(=O)Nc2ccc(OCCCN(C)C)c(-c3ccnn3C)c2)c1] | 1-[4-(3-Dimethylamino- propoxy)-3-(2-methyl-2H- pyrazol-3-yl)-phenyl]-3-(2- fluoro-5-methyl-phenyl)-urea |
| 140 | 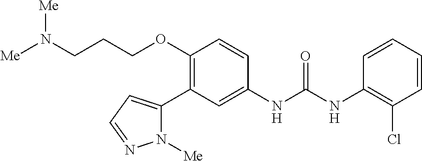 [SMILES: CN(C)CCCOc1ccc(NC(=O)Nc2ccccc2Cl)cc1-c1ccnn1C] | 1-(2-Chloro-phenyl)-3-[4-(3- dimethylamino-propoxy)-3-(2- methyl-2H-pyrazol-3-yl)- phenyl]-urea |
| 141 | 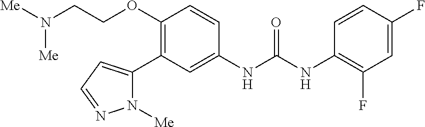 [SMILES: CN(C)CCOc1ccc(NC(=O)Nc2ccc(F)cc2F)cc1-c1ccnn1C] | 1-(2,4-Difluoro-phenyl)-3-[4-(2- dimethylamino-ethoxy)-3-(2- methyl-2H-pyrazol-3-yl)- phenyl]-urea |
| 142 | 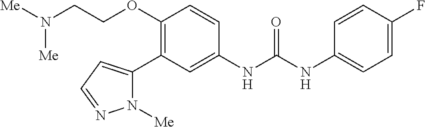 [SMILES: CN(C)CCOc1ccc(NC(=O)Nc2ccc(F)cc2)cc1-c1ccnn1C] | 1-[4-(2-Dimethylamino-ethoxy)- 3-(2-methyl-2H-pyrazol-3-yl)- phenyl]-3-(4-fluoro-phenyl)-urea |
| 143 | 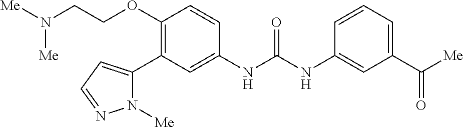 [SMILES: CC(=O)c1cccc(NC(=O)Nc2ccc(OCCN(C)C)c(-c3ccnn3C)c2)c1] | 1-(3-Acetyl-phenyl)-3-[4-(2- dimethylamino-ethoxy)-3-(2- methyl-2H-pyrazol-3-yl)- phenyl]-urea |
| 144 | 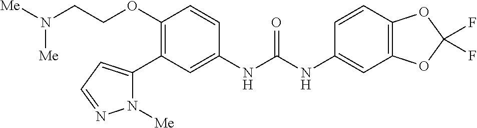 [SMILES: CN(C)CCOc1ccc(NC(=O)Nc2ccc3c(c2)OC(F)(F)O3)cc1-c1ccnn1C] | 1-(2,2-Difluoro- benzo[1,3]dioxol-5-yl)-3-[4-(2- dimethylamino-ethoxy)-3-(2- methyl-2H-pyrazol-3-yl)- phenyl]-urea |
| 145 | 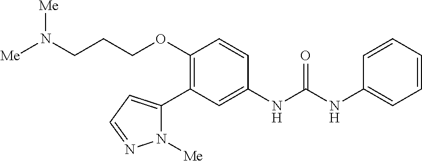 [SMILES: CN(C)CCCOc1ccc(NC(=O)Nc2ccccc2)cc1-c1ccnn1C] | 1-[4-(3-Dimethylamino- propoxy)-3-(2-methyl-2H- pyrazol-3-yl)-phenyl]-3-phenyl- urea |
| 146 | 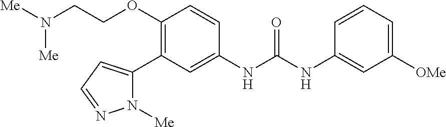 [SMILES: COc1cccc(NC(=O)Nc2ccc(OCCN(C)C)c(-c3ccnn3C)c2)c1] | 1-[4-(2-Dimethylamino-ethoxy)- 3-(2-methyl-2H-pyrazol-3-yl)- phenyl]-3-(3-methoxy-phenyl)- urea |
| 147 | 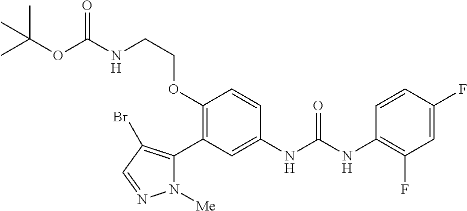 [SMILES: Cn1ncc(Br)c1-c1cc(NC(=O)Nc2ccc(F)cc2F)ccc1OCCNC(=O)OC(C)(C)C] | (2-{2-(4-Bromo-2-methyl-2H- pyrazol-3-yl)-4-[3-(2,4-difluoro- phenyl)-ureido]-phenoxy}- ethyl)-carbamic acid tert-butyl ester |
| 148 | 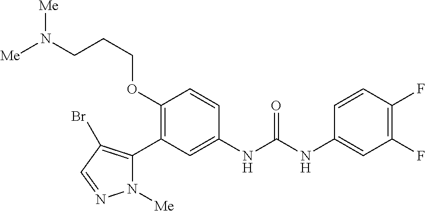 [SMILES: CN(C)CCCOc1ccc(NC(=O)Nc2ccc(F)c(F)c2)cc1-c1c(Br)cnn1C] | 1-[3-(4-Bromo-2-methyl-2H- pyrazol-3-yl)-4-(3- dimethylamino-propoxy)- phenyl]-3-(3,4-difluoro-phenyl)- urea |
| 149 | 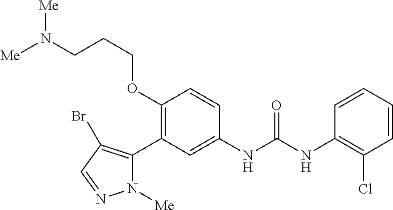 [SMILES: CN(C)CCCOc1ccc(NC(=O)Nc2ccccc2Cl)cc1-c1c(Br)cnn1C] | 1-[3-(4-Bromo-2-methyl-2H- pyrazol-3-yl)-4-(3- dimethylamino-propoxy)- phenyl]-3-(2-chloro-phenyl)-urea |
| 150 | 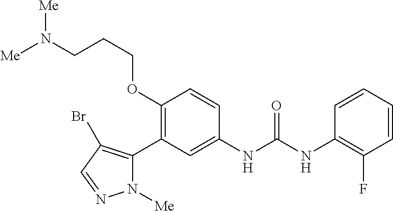 [SMILES: CN(C)CCCOc1ccc(NC(=O)Nc2ccccc2F)cc1-c1c(Br)cnn1C] | 1-[3-(4-Bromo-2-methyl-2H- pyrazol-3-yl)-4-(3- dimethylamino-propoxy)- phenyl]-3-(2-fluoro-phenyl)-urea |
| 151 | 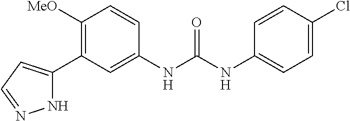 [SMILES: COc1ccc(NC(=O)Nc2ccc(Cl)cc2)cc1-c1ccn[nH]1] | 1-(4-Chloro-phenyl)-3-[4- methoxy-3-(2H-pyrazol-3-yl)- phenyl]-urea |
| 152 | 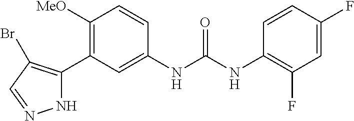 [SMILES: COc1ccc(NC(=O)Nc2ccc(F)cc2F)cc1-c1[nH]ncc1Br] | 1-[3-(4-Bromo-2H-pyrazol-3- yl)-4-methoxy-phenyl]-3-(2,4- difluoro-phenyl)-urea |
| 153 | 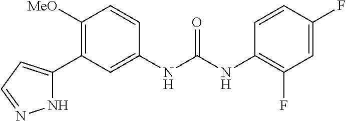 [SMILES: COc1ccc(NC(=O)Nc2ccc(F)cc2F)cc1-c1ccn[nH]1] | 1-(2,4-Difluoro-phenyl)-3-[4- methoxy-3-(2H-pyrazol-3-yl)- phenyl]-urea |
| 154 | 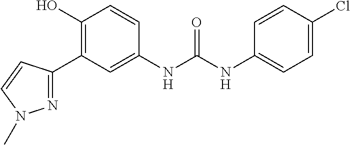 [SMILES: Cn1ccc(-c2cc(NC(=O)Nc3ccc(Cl)cc3)ccc2O)n1] | 1-(4-Chloro-phenyl)-3-[4- hydroxy-3-(1-methyl-1H- pyrazol-3-yl)-phenyl]-urea |
| 155 | 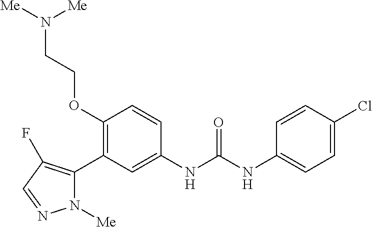 [SMILES: CN(C)CCOc1ccc(NC(=O)Nc2ccc(Cl)cc2)cc1-c1c(F)cnn1C] | 1-(4-Chloro-phenyl)-3-[4-(2- dimethylamino-ethoxy)-3-(4- fluoro-2-methyl-2H-pyrazol-3- yl)-phenyl]-urea |
| 156 | 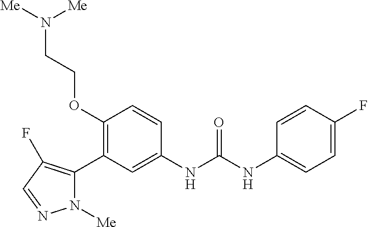 [SMILES: CN(C)CCOc1ccc(NC(=O)Nc2ccc(F)cc2)cc1-c1c(F)cnn1C] | 1-[4-(2-Dimethylamino-ethoxy)- 3-(4-fluoro-2-methyl-2H- pyrazol-3-yl)-phenyl]-3-(4- fluoro-phenyl)-urea |
| 157 | 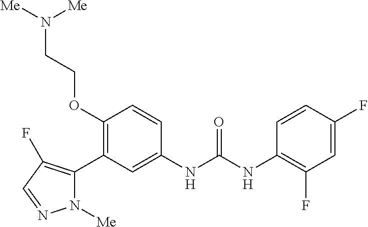 [SMILES: CN(C)CCOc1ccc(NC(=O)Nc2ccc(F)cc2F)cc1-c1c(F)cnn1C] | 1-(2,4-Difluoro-phenyl)-3-[4-(2- dimethylamino-ethoxy)-3-(4- fluoro-2-methyl-2H-pyrazol-3- yl)-phenyl]-urea |
| 158 | 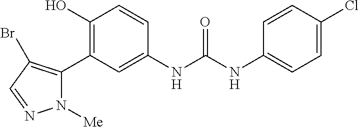 [SMILES: Cn1ncc(Br)c1-c1cc(NC(=O)Nc2ccc(Cl)cc2)ccc1O] | 1-(4-Chloro-2-hydroxy-phenyl)- 3-[4-(2-dimethylamino-ethoxy)- 3-(4-fluoro-2-methyl-2H- pyrazol-3-yl)-phenyl]-urea |
| 159 | 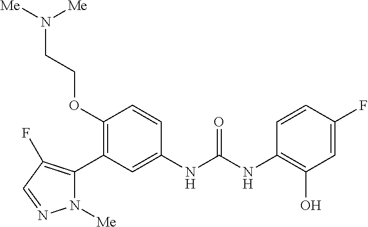 [SMILES: CN(C)CCOc1ccc(NC(=O)Nc2ccc(F)cc2O)cc1-c1c(F)cnn1C] | 1-[4-(2-Dimethylamino-ethoxy)- 3-(4-fluoro-2-methyl-2H- pyrazol-3-yl)-phenyl]-3-(4- fluoro-2-hydroxy-phenyl)-urea |
| 160 | 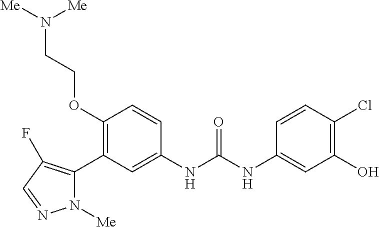 [SMILES: CN(C)CCOc1ccc(NC(=O)Nc2ccc(Cl)c(O)c2)cc1-c1c(F)cnn1C] | 1-(4-Chloro-3-hydroxy-phenyl)- 3-[4-(2-dimethylamino-ethoxy)- 3-(4-fluoro-2-methyl-2H- pyrazol-3-yl)-phenyl]-urea |
| 161 | 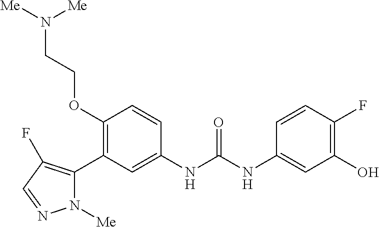 [SMILES: CN(C)CCOc1ccc(NC(=O)Nc2ccc(F)c(O)c2)cc1-c1c(F)cnn1C] | 1-[4-(2-Dimethylamino-ethoxy)- 3-(4-fluoro-2-methyl-2H- pyrazol-3-yl)-phenyl]-3-(4- fluoro-3-hydroxy-phenyl)-urea |
| 162 | 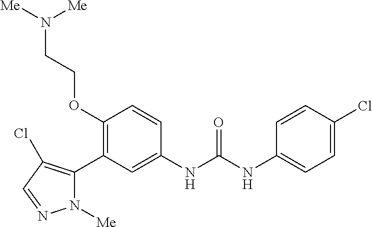 [SMILES: CN(C)CCOc1ccc(NC(=O)Nc2ccc(Cl)cc2)cc1-c1c(Cl)cnn1C] | 1-[3-(4-Chloro-2-methyl-2H- pyrazol-3-yl)-4-(2- dimethylamino-ethoxy)-phenyl]- 3-(4-chloro-phenyl)-urea |
| 163 | 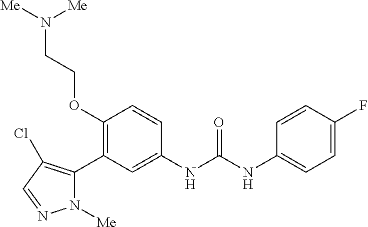 [SMILES: CN(C)CCOc1ccc(NC(=O)Nc2ccc(F)cc2)cc1-c1c(Cl)cnn1C] | 1-[3-(4-Chloro-2-methyl-2H- pyrazol-3-yl)-4-(2- dimethylamino-ethoxy)-phenyl]- 3-(4-fluoro-phenyl)-urea |
| 164 | 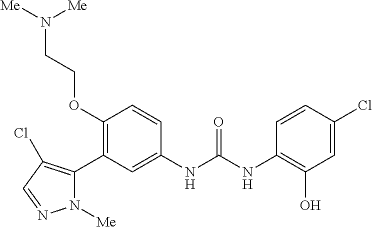 [SMILES: CN(C)CCOc1ccc(NC(=O)Nc2ccc(Cl)cc2O)cc1-c1c(Cl)cnn1C] | 1-(4-Chloro-2-hydroxy-phenyl)- 3-[3-(4-chloro-2-methyl-2H- pyrazol-3-yl)-4-(2- dimethylamino-ethoxy)-phenyl]- urea |
| 165 | 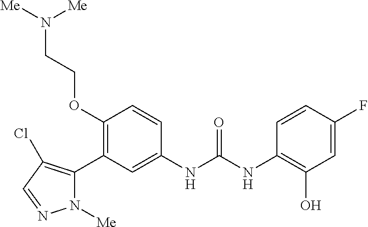 [SMILES: CN(C)CCOc1ccc(NC(=O)Nc2ccc(F)cc2O)cc1-c1c(Cl)cnn1C] | 1-[3-(4-Chloro-2-methyl-2H- pyrazol-3-yl)-4-(2- dimethylamino-ethoxy)-phenyl]- 3-(4-fluoro-2-hydroxy-phenyl)- urea |
| 166 | 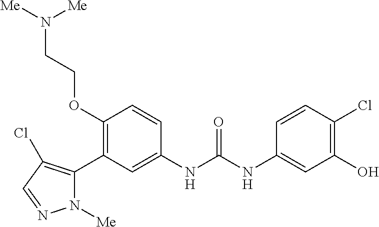 [SMILES: CN(C)CCOc1ccc(NC(=O)Nc2ccc(Cl)c(O)c2)cc1-c1c(Cl)cnn1C] | 1-(4-Chloro-3-hydroxy-phenyl)- 3-[3-(4-chloro-2-methyl-2H- pyrazol-3-yl)-4-(2- dimethylamino-ethoxy)-phenyl]- urea |
| 167 | 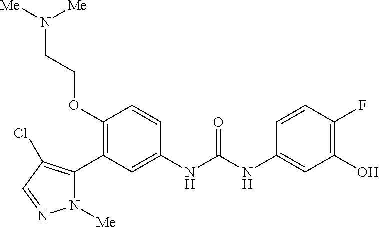 [SMILES: CN(C)CCOc1ccc(NC(=O)Nc2ccc(F)c(O)c2)cc1-c1c(Cl)cnn1C] | 1-[3-(4-Chloro-2-methyl-2H- pyrazol-3-yl)-4-(2- dimethylamino-ethoxy)-phenyl]- 3-(4-fluoro-3-hydroxy-phenyl)- urea |
| 168 | 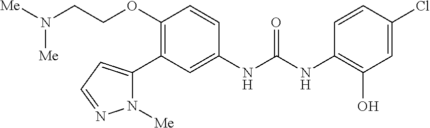 [SMILES: CN(C)CCOc1ccc(NC(=O)Nc2ccc(Cl)cc2O)cc1-c1ccnn1C] | 1-(4-Chloro-2-hydroxy-phenyl)- 3-[4-(2-Dimethylamino-ethoxy)- 3-(2-methyl-2H-pyrazol-3-yl)- phenyl]-urea |
| 169 | 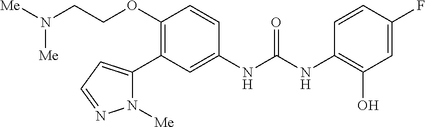 [SMILES: CN(C)CCOc1ccc(NC(=O)Nc2ccc(F)cc2O)cc1-c1ccnn1C] | 1-[4-(2-Dimethylamino-ethoxy)- 3-(2-methyl-2H-pyrazol-3-yl)- phenyl]-3-(4-fluoro-2-hydroxy- phenyl)-urea |
| 170 | 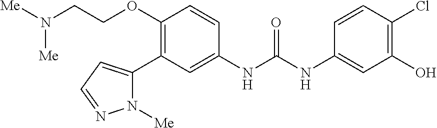 [SMILES: CN(C)CCOc1ccc(NC(=O)Nc2ccc(Cl)c(O)c2)cc1-c1ccnn1C] | 1-(4-Chloro-3-hydroxy-phenyl)- 3-[4-(2-dimethylamino-ethoxy)- 3-(2-methyl-2H-pyrazol-3-yl)- phenyl]-urea |
| 171 | 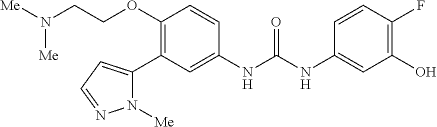 [SMILES: CN(C)CCOc1ccc(NC(=O)Nc2ccc(F)c(O)c2)cc1-c1ccnn1C] | 1-[4-(2-Dimethylamino-ethoxy)- 3-(2-methyl-2H-pyrazol-3-yl)- phenyl]-3-(4-fluoro-3-hydroxy- phenyl)-urea |
| 172 | 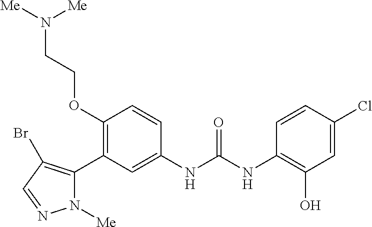 [SMILES: CN(C)CCOc1ccc(NC(=O)Nc2ccc(Cl)cc2O)cc1-c1c(Br)cnn1C] | 1-[3-(4-Bromo-2-methyl-2H- pyrazol-3-yl)-4-(2- dimethylamino-ethoxy)-phenyl]- 3-(4-chloro-2-hydroxy-phenyl)- urea |
| 173 | 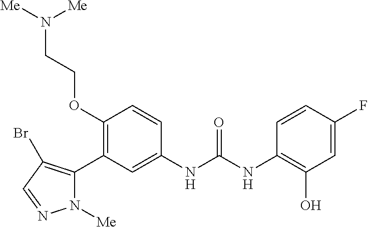 [SMILES: CN(C)CCOc1ccc(NC(=O)Nc2ccc(F)cc2O)cc1-c1c(Br)cnn1C] | 1-[3-(4-Bromo-2-methyl-2H- pyrazol-3-yl)-4-(2- dimethylamino-ethoxy)-phenyl]- 3-(4-fluoro-2-hydroxy-phenyl)- urea |
| 174 | 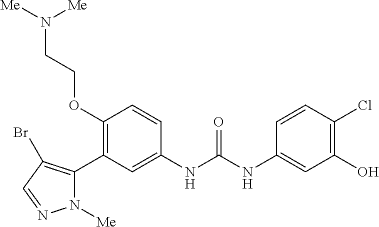 [SMILES: CN(C)CCOc1ccc(NC(=O)Nc2ccc(Cl)c(O)c2)cc1-c1c(Br)cnn1C] | 1-[3-(4-Bromo-2-methyl-2H- pyrazol-3-yl)-4-(2- dimethylamino-ethoxy)-phenyl]- 3-(4-chloro-3-hydroxy-phenyl)- urea |
| 175 | 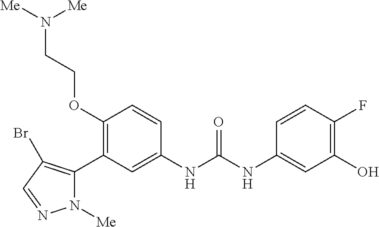 [SMILES: CN(C)CCOc1ccc(NC(=O)Nc2ccc(F)c(O)c2)cc1-c1c(Br)cnn1C] | 1-[3-(4-Bromo-2-methyl-2H- pyrazol-3-yl)-4-(2- dimethylamino-ethoxy)-phenyl]- 3-(4-fluoro-3-hydroxy-phenyl)- urea |
| 176 | 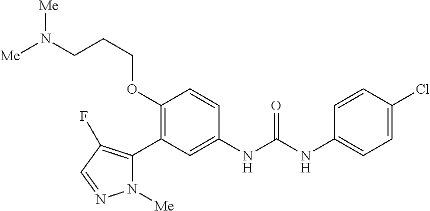 [SMILES: CN(C)CCCOc1ccc(NC(=O)Nc2ccc(Cl)cc2)cc1-c1c(F)cnn1C] | 1-(4-Chloro-phenyl)-3-[4-(3- dimethylamino-propoxy)-3-(4- fluoro-2-methyl-2H-pyrazol-3- yl)-phenyl]-urea |
| 177 | 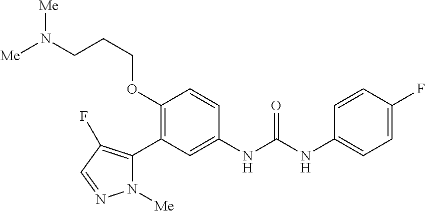 [SMILES: CN(C)CCCOc1ccc(NC(=O)Nc2ccc(F)cc2)cc1-c1c(F)cnn1C] | 1-[4-(3-Dimethylamino- propoxy)-3-(4-fluoro-2-methyl- 2H-pyrazol-3-yl)-phenyl]-3-(4- fluoro-phenyl)-urea |
| 178 | 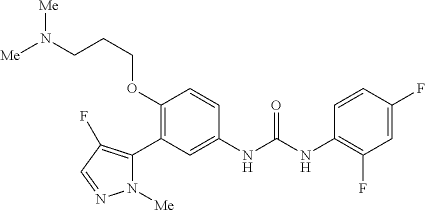 [SMILES: CN(C)CCCOc1ccc(NC(=O)Nc2ccc(F)cc2F)cc1-c1c(F)cnn1C] | 1-(2,4-Difluoro-phenyl)-3-[4-(3- dimethylamino-propoxy)-3-(4- fluoro-2-methyl-2H-pyrazol-3- yl)-phenyl]-urea |
| 179 | 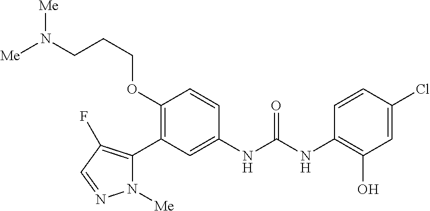 [SMILES: CN(C)CCCOc1ccc(NC(=O)Nc2ccc(Cl)cc2O)cc1-c1c(F)cnn1C] | 1-(4-Chloro-2-hydroxy-phenyl)- 3-[4-(3-dimethylamino- propoxy)-3-(4-fluoro-2-methyl- 2H-pyrazol-3-yl)-phenyl]-urea |
| 180 | 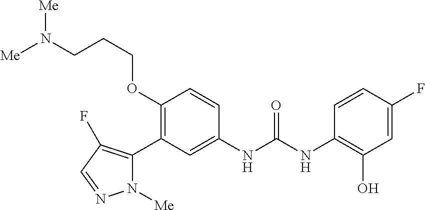 [SMILES: CN(C)CCCOc1ccc(NC(=O)Nc2ccc(F)cc2O)cc1-c1c(F)cnn1C] | 1-[4-(3-Dimethylamino- propoxy)-3-(4-fluoro-2-methyl- 2H-pyrazol-3-yl)-phenyl]-3-(4- fluoro-2-hydroxy-phenyl)-urea |
| 181 | 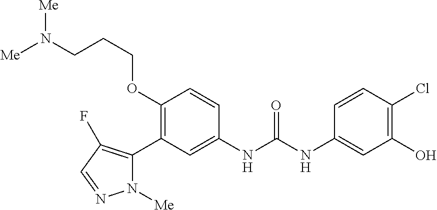 [SMILES: CN(C)CCCOc1ccc(NC(=O)Nc2ccc(Cl)c(O)c2)cc1-c1c(F)cnn1C] | 1-(4-Chloro-3-hydroxy-phenyl)- 3-[4-(3-dimethylamino- propoxy)-3-(4-fluoro-2-methyl- 2H-pyrazol-3-yl)-phenyl]-urea |
| 182 | 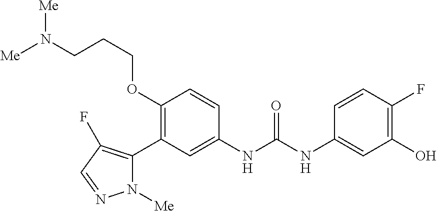 [SMILES: CN(C)CCCOc1ccc(NC(=O)Nc2ccc(F)c(O)c2)cc1-c1c(F)cnn1C] | 1-[4-(3-Dimethylamino- propoxy)-3-(4-fluoro-2-methyl- 2H-pyrazol-3-yl)-phenyl]-3-(4- fluoro-3-hydroxy-phenyl)-urea |
| 183 | 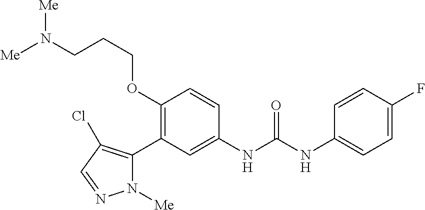 [SMILES: CN(C)CCCOc1ccc(NC(=O)Nc2ccc(F)cc2)cc1-c1c(Cl)cnn1C] | 1-[3-(4-Chloro-2-methyl-2H- pyrazol-3-yl)-4-(3- dimethylamino-propoxy)- phenyl]-3-(4-fluoro-phenyl)-urea |
| 184 | 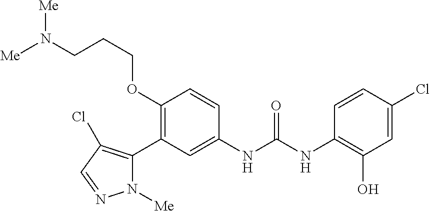 [SMILES: CN(C)CCCOc1ccc(NC(=O)Nc2ccc(Cl)cc2O)cc1-c1c(Cl)cnn1C] | 1-(4-Chloro-2-hydroxy-phenyl)- 3-[3-(4-chloro-2-methyl-2H- pyrazol-3-yl)-4-(3- dimethylamino-propoxy)- phenyl]-urea |
| 185 | 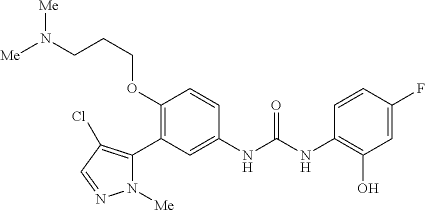 [SMILES: CN(C)CCCOc1ccc(NC(=O)Nc2ccc(F)cc2O)cc1-c1c(Cl)cnn1C] | 1-[3-(4-Chloro-2-methyl-2H- pyrazol-3-yl)-4-(3- dimethylamino-propoxy)- phenyl]-3-(4-fluoro-2-hydroxy- phenyl)-urea |
| 186 | 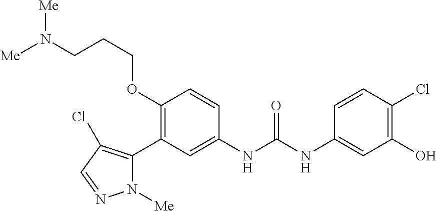 [SMILES: CN(C)CCCOc1ccc(NC(=O)Nc2ccc(Cl)c(O)c2)cc1-c1c(Cl)cnn1C] | 1-(4-Chloro-3-hydroxy-phenyl)- 3-[3-(4-chloro-2-methyl-2H- pyrazol-3-yl)-4-(3- dimethylamino-propoxy)- phenyl]-urea |
| 187 | 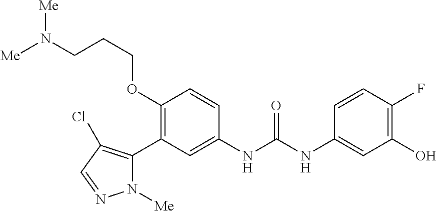 [SMILES: CN(C)CCCOc1ccc(NC(=O)Nc2ccc(F)c(O)c2)cc1-c1c(Cl)cnn1C] | 1-[3-(4-Chloro-2-methyl-2H- pyrazol-3-yl)-4-(3- dimethylamino-propoxy)- phenyl]-3-(4-fluoro-3-hydroxy- phenyl)-urea |
| 188 | 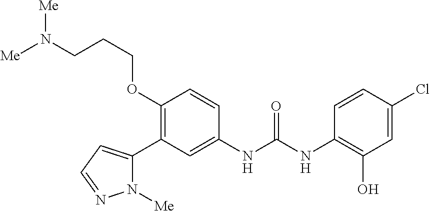 [SMILES: CN(C)CCCOc1ccc(NC(=O)Nc2ccc(Cl)cc2O)cc1-c1ccnn1C] | 1-(4-Chloro-2-hydroxy-phenyl)- 3-[4-(3-dimethylamino- propoxy)-3-(2-methyl-2H- pyrazol-3-yl)-phenyl]-urea |
| 189 | 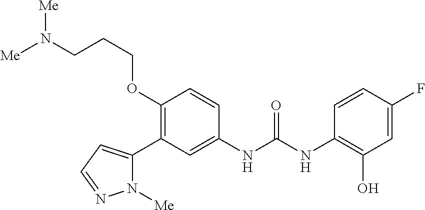 [SMILES: CN(C)CCCOc1ccc(NC(=O)Nc2ccc(F)cc2O)cc1-c1ccnn1C] | 1-[4-(3-Dimethylamino- propoxy)-3-(2-methyl-2H- pyrazol-3-yl)-phenyl]-3-(4- fluoro-2-hydroxy-phenyl)-urea |
| 190 | 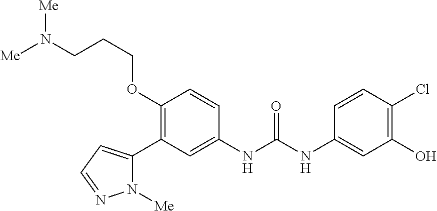 [SMILES: CN(C)CCCOc1ccc(NC(=O)Nc2ccc(Cl)c(O)c2)cc1-c1ccnn1C] | 1-(4-Chloro-3-hydroxy-phenyl)- 3-[4-(3-dimethylamino- propoxy)-3-(2-methyl-2H- pyrazol-3-yl)-phenyl]-urea |
| 191 | 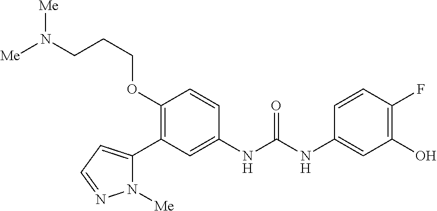 [SMILES: CN(C)CCCOc1ccc(NC(=O)Nc2ccc(F)c(O)c2)cc1-c1ccnn1C] | 1-[4-(3-Dimethylamino- propoxy)-3-(2-methyl-2H- pyrazol-3-yl)-phenyl]-3-(4- fluoro-3-hydroxy-phenyl)-urea |
| 192 | 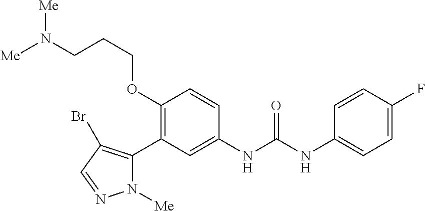 [SMILES: CN(C)CCCOc1ccc(NC(=O)Nc2ccc(F)cc2)cc1-c1c(Br)cnn1C] | 1-[3-(4-Bromo-2-methyl-2H- pyrazol-3-yl)-4-(3- dimethylamino-propoxy)- phenyl]-3-(4-fluoro-phenyl)-urea |
| 193 | 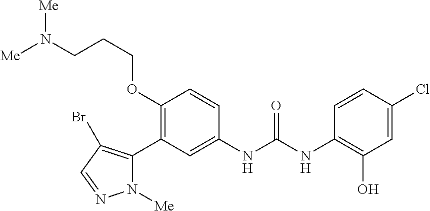 [SMILES: CN(C)CCCOc1ccc(NC(=O)Nc2ccc(Cl)cc2O)cc1-c1c(Br)cnn1C] | 1-[3-(4-Bromo-2-methyl-2H- pyrazol-3-yl)-4-(3- dimethylamino-propoxy)- phenyl]-3-(4-chloro-2-hydroxy- phenyl)-urea |
| 194 | 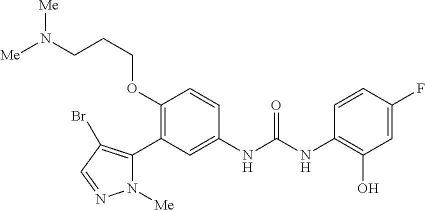 [SMILES: CN(C)CCCOc1ccc(NC(=O)Nc2ccc(F)cc2O)cc1-c1c(Br)cnn1C] | 1-[3-(4-Bromo-2-methyl-2H- pyrazol-3-yl)-4-(3- dimethylamino-propoxy)- phenyl]-3-(4-fluoro-2-hydroxy- phenyl)-urea |
| 195 | 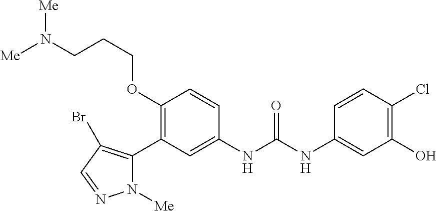 [SMILES: CN(C)CCCOc1ccc(NC(=O)Nc2ccc(Cl)c(O)c2)cc1-c1c(Br)cnn1C] | 1-[3-(4-Bromo-2-methyl-2H- pyrazol-3-yl)-4-(3- dimethylamino-propoxy)- phenyl]-3-(4-chloro-3-hydroxy- phenyl)-urea |
| 196 | 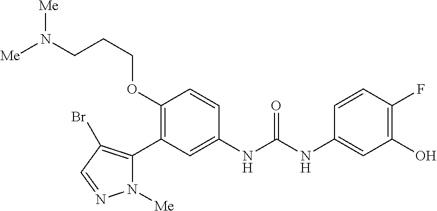 [SMILES: CN(C)CCCOc1ccc(NC(=O)Nc2ccc(F)c(O)c2)cc1-c1c(Br)cnn1C] | 1-[3-(4-Bromo-2-methyl-2H- pyrazol-3-yl)-4-(3- dimethylamino-propoxy)- phenyl]-3-(4-fluoro-3-hydroxy- phenyl)-urea |

Additionally, compounds, such as Formula (I) and related Formulae, encompass all pharmaceutically acceptable salts, solvates, and particularly hydrates, thereof.

The compounds of the Formula (I) may be prepared according to the schemes set forth in U.S. Pat. No. 8,754,238.

Reference to the above compounds also encompasses diastereomers as well as optical isomers, e.g. mixtures of enantiomers including racemic mixtures, as well as individual enantiomers and diastereomers, which arise as a consequence of structural asymmetry in certain compounds of the invention. Separation of the individual isomers or selective synthesis of the individual isomers is accomplished by application of various methods which are well known to practitioners in the art.

### Chemical Group, Moiety or Radical:

The term “C₁₋₆ acyl” denotes a C₁₋₆ alkyl radical attached to a carbonyl wherein the definition of alkyl has the same definition as described herein; some examples include but not limited to, acetyl, propionyl, n-butanoyl, iso-butanoyl, sec-butanoyl, t-butanoyl (i.e., pivaloyl), pentanoyl and the like.

The term “C₁₋₆ acyloxy” denotes an acyl radical attached to an oxygen atom wherein acyl has the same definition has described herein; some examples include but not limited to acetyloxy, propionyloxy, butanoyloxy, iso-butanoyloxy, sec-butanoyloxy, t-butanoyloxy and the like.

The term “C₂₋₆ alkenyl” denotes a radical containing 2 to 6 carbons wherein at least one carbon-carbon double bond is present, some embodiments are 2 to 4 carbons, some embodiments are 2 to 3 carbons, and some embodiments have 2 carbons. Both E and Z isomers are embraced by the term “alkenyl.” Furthermore, the term “alkenyl” includes di- and tri-alkenyls. Accordingly, if more than one double bond is present then the bonds may be all E or Z or a mixtures of E and Z. Examples of an alkenyl include vinyl, allyl, 2-butenyl, 3-butenyl, 2-pentenyl, 3-pentenyl, 4-pentenyl, 2-hexenyl, 3-hexenyl, 4-hexenyl, 5-hexanyl, 2,4-hexadienyl and the like.

The term “C₁₋₆ alkoxy” as used herein denotes a radical alkyl, as defined herein, attached directly to an oxygen atom. Examples include methoxy, ethoxy, n-propoxy, iso-propoxy, n-butoxy, t-butoxy, iso-butoxy, sec-butoxy and the like.

The term “C₁₋₈ alkyl” denotes a straight or branched carbon radical containing 1 to 8 carbons, some embodiments are 1 to 6 carbons, some embodiments are 1 to 4 carbons, some embodiments are 1 to 3 carbons, and some embodiments are 1 or 2 carbons. Examples of an alkyl include, but not limited to, methyl, ethyl, n-propyl, iso-propyl, n-butyl, sec-butyl, iso-butyl, t-butyl, pentyl, iso-pentyl, t-pentyl, neo-pentyl, 1-methylbutyl [i.e., —CH(CH₃)CH₂CH₂CH₃], 2-methylbutyl [i.e., —CH₂CH(CH₃)CH₂CH₃], n-hexyl and the like.

The term “C₁₋₆ alkylcarboxamido” or “C₁₋₆ alkylcarboxamide” denotes a single C₁₋₆ alkyl group attached to the nitrogen of an amide group, wherein alkyl has the same definition as found herein. The C₁₋₆ alkylcarboxamido may be represented by the following:

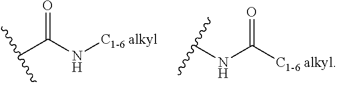

Examples include, but not limited to, N-methylcarboxamide, N-ethylcarboxamide, N-n-propylcarboxamide, N-iso-propylcarboxamide, N-n-butylcarboxamide, N-sec-butylcarboxamide, N-iso-butylcarboxamide, N-t-butylcarboxamide and the like.

The term “C₁₋₃ alkylene” refers to a C₁₋₃ divalent straight carbon group. In some embodiments C₁₋₃ alkylene refers to, for example, —CH₂—, —CH₂CH₂—, —CH₂CH₂CH₂—, and the like. In some embodiments, C₁₋₃ alkylene refers to —CH—, —CHCH₂—, —CHCH₂CH₂—, and the like wherein these examples relate generally to the variable or claim element “Q”.

The term “C₁₋₆ alkylimino” denotes a C₁₋₆ alkyl radical attached directly to the carbon of the —C(═NH)— group wherein the definition of alkyl has the same definition as described herein; some examples include but not limited to, 1-imino-ethyl [i.e., —C(═NH)CH₃], 1-imino-propyl [i.e., —C(═NH)CH₂CH₃], 1-imino-2-methyl-propyl [i.e., —C(═NH)CH(CH₃)₂], and the like.

The term “C₁₋₆ alkylsulfinyl” denotes a C₁₋₆ alkyl radical attached to a sulfoxide radical of the formula: —S(O)— wherein the alkyl radical has the same definition as described herein. Examples include, but not limited to, methylsulfinyl, ethylsulfinyl, n-propylsulfinyl, iso-propylsulfinyl, n-butylsulfinyl, sec-butylsulfinyl, iso-butylsulfinyl, t-butylsulfinyl, and the like.

The term “C₁₋₆ alkylsulfonamide” refers to the groups

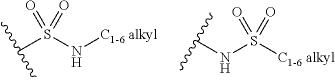

wherein C₁₋₆ alkyl has the same definition as described herein.

The term “C₁₋₆ alkylsulfonyl” denotes a C₁₋₆ alkyl radical attached to a sulfone radical of the formula: —S(O)₂— wherein the alkyl radical has the same definition as described herein. Examples include, but not limited to, methylsulfonyl, ethylsulfonyl, n-propyl sulfonyl, iso-propyl sulfonyl, n-butylsulfonyl, sec-butyl sulfonyl, iso-butyl sulfonyl, t-butylsulfonyl, and the like.

The term “C₁₋₆ alkylthio” denotes a C₁₋₆ alkyl radical attached to a sulfide of the formula: —S— wherein the alkyl radical has the same definition as described herein. Examples include, but not limited to, methylsulfanyl (i.e., CH₃S—), ethylsulfanyl, n-propylsulfanyl, iso-propylsulfanyl, n-butylsulfanyl, sec-butyl sulfanyl, iso-butylsulfanyl, t-butylsulfanyl, and the like.

The term “C₁₋₆ alkylthiocarboxamide” denotes a thioamide of the following formulae:

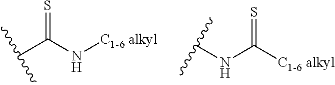

wherein C₁₋₄ alkyl has the same definition as described herein.

The term “C₁₋₆ alkylthioureyl” denotes the group of the formula:

—NC(S)N— wherein one are both of the nitrogens are substituted with the same or different C₁₋₆ alkyl groups and alkyl has the same definition as described herein. Examples of an alkylthioureyl include, but not limited to, CH₃NHC(S)NH—, NH₂C(S)NCH₃—, (CH₃)₂N(S)NH—, (CH₃)₂N(S)NH—, (CH₃)₂N(S)NCH₃—, CH₃CH₂NHC(S)NH—, CH₃CH₂NHC(S)NCH₃—, and the like.

The term “C₁₋₆ alkylureyl” denotes the group of the formula: —NC(O)N— wherein one are both of the nitrogens are substituted with the same or different C₁₋₆ alkyl group wherein alkyl has the same definition as described herein. Examples of an alkylureyl include, but not limited to, CH₃NHC(O)NH—, NH₂C(O)NCH₃—, (CH₃)₂NC(O)NH—, (CH₃)₂NC(O)NH—, (CH₃)₂NC(O)NCH₃—, CH₃CH₂NHC(O)NH—, CH₃CH₂NHC(O)NCH₃—, and the like.

The term “C₂₋₆ alkynyl” denotes a radical containing 2 to 6 carbons and at least one carbon-carbon triple bond, some embodiments are 2 to 4 carbons, some embodiments are 2 to 3 carbons, and some embodiments have 2 carbons. Examples of an alkynyl include, but not limited to, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like. The term “alkynyl” includes di- and tri-ynes.

The term “amino” denotes the group —NH₂.

The term “C₁₋₆ alkylamino” denotes one alkyl radical attached to an amino radical wherein the alkyl radical has the same meaning as described herein. Some examples include, but not limited to, methylamino, ethylamino, n-propylamino, iso-propylamino, n-butylamino, sec-butylamino, iso-butylamino, t-butylamino, and the like. Some embodiments are “C₁₋₂ alkylamino.”

The term “aryl” denotes an aromatic ring radical containing 6 to 10 ring carbons. Examples include phenyl and naphthyl.

The term “arylalkyl” defines a C₁-C₄ alkylene, such as —CH₂—, —CH₂CH₂— and the like, which is further substituted with an aryl group. Examples of an “arylalkyl” include benzyl, phenethylene and the like.

The term “arylcarboxamido” denotes a single aryl group attached to the nitrogen of an amide group, wherein aryl has the same definition as found herein. The example is N-phenylcarboxamide.

The term “arylureyl” denotes the group —NC(O)N— where one of the nitrogens are substituted with an aryl.

The term “benzyl” denotes the group —CH₂C₆H₅.

The term “carbo-C₁₋₆-alkoxy” refers to a C₁₋₆ alkyl ester of a carboxylic acid, wherein the alkyl group is as defined herein. Examples include, but not limited to, carbomethoxy, carboethoxy, carbopropoxy, carboisopropoxy, carbobutoxy, carbo-sec-butoxy, carbo-iso-butoxy, carbo-t-butoxy, carbo-n-pentoxy, carbo-iso-pentoxy, carbo-t-pentoxy, carbo-neo-pentoxy, carbo-n-hexyloxy, and the like.

The term “carboxamide” refers to the group —CONH₂.

The term “carboxy” or “carboxyl” denotes the group —CO₂H; also referred to as a carboxylic acid group.

The term “cyano” denotes the group —CN.

The term “C₄₋₇ cycloalkenyl” denotes a non-aromatic ring radical containing 4 to 7 ring carbons and at least one double bond; some embodiments contain 4 to 6 carbons; some embodiments contain 4 to 5 carbons; some embodiments contain 4 carbons. Examples include cyclobutenyl, cyclopentenyl, cyclopentenyl, cyclohexenyl, and the like.

The term “C₃₋₇ cycloalkyl” denotes a saturated ring radical containing 3 to 7 carbons; some embodiments contain 3 to 6 carbons; some embodiments contain 3 to 5 carbons; some embodiments contain 5 to 7 carbons; some embodiments contain 3 to 4 carbons. Examples include cyclopropyl, cyclobutyl, cyclopentyl, cyclopenyl, cyclohexyl, cycloheptyl and the like.

The term “C₂₋₈ dialkylamino” denotes an amino substituted with two of the same or different C₁₋₄ alkyl radicals wherein alkyl radical has the same definition as described herein. Some examples include, but not limited to, dimethylamino, methylethylamino, diethylamino, methylpropylamino, methylisopropylamino, ethylpropyl amino, ethylisopropylamino, dipropylamino, propylisopropylamino and the like. Some embodiments are “C₂₋₄ dialkylamino.”

The term “C₂₋₈ dialkylcarboxamido” or “C₂₋₈ dialkylcarboxamide” denotes two alkyl radicals, that are the same or different, attached to an amide group, wherein alkyl has the same definition as described herein. A C₂₋₈ dialkylcarboxamido may be represented by the following groups:

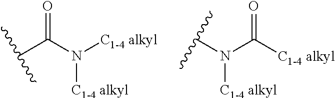

wherein C₁₋₄ has the same definition as described herein. Examples of a dialkylcarboxamide include, but not limited to, N,N-dimethylcarboxamide, N-methyl-N-ethylcarboxamide, N,N-diethylcarboxamide, N-methyl-N-isopropylcarboxamide, and the like.

The term “C₂₋₈ dialkylsulfonamide” refers to one of the following groups shown below:

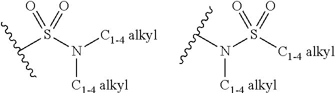

wherein C₁₋₄ has the same definition as described herein, for example but not limited to, methyl, ethyl, n-propyl, isopropyl, and the like.

The term “C₂₋₈ dialkylthiocarboxamido” or “C₂₋₈ dialkylthiocarboxamide” denotes two alkyl radicals, that are the same or different, attached to a thioamide group, wherein alkyl has the same definition as described herein. A C₂₋₈ dialkylthiocarboxamido or C₂₋₈ dialkylthiocarboxamide may be represented by the following groups:

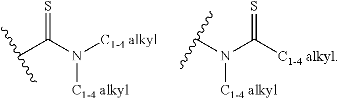

Examples of a dialkylthiocarboxamide include, but not limited to, N,N-dimethylthiocarboxamide, N-methyl-N-ethylthiocarboxamide and the like.

The term “ethynylene” refers to the carbon-carbon triple bond group as represented below:

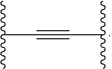 [SMILES: *C#C*]

The term “formyl” refers to the group —CHO.

The term “C₁₋₆ haloalkoxy” denotes a haloalkyl, as defined herein, which is directly attached to an oxygen atom. Examples include, but not limited to, difluoromethoxy, trifluoromethoxy, 2,2,2-trifluoroethoxy, pentafluoroethoxy and the like.

The term “C₁₋₆ haloalkyl” denotes an C₁₋₆ alkyl group, defined herein, wherein the alkyl is substituted with one halogen up to fully substituted and a fully substituted C₁₋₆ haloalkyl can be represented by the formula CₙL₂ₙ₊₁ wherein L is a halogen and “n” is 1, 2, 3 or 4; when more than one halogen is present then they may be the same or different and selected from the group consisting of F, Cl, Br and I, preferably F. Examples of C₁₋₄ haloalkyl groups include, but not limited to, fluoromethyl, difluoromethyl, trifluoromethyl, chlorodifluoromethyl, 2,2,2-trifluoroethyl, pentafluoroethyl and the like.

The term “C₁₋₆ haloalkylcarboxamide” denotes an alkylcarboxamide group, defined herein, wherein the alkyl is substituted with one halogen up to fully substituted represented by the formula CₙL₂ₙ₊₁ wherein L is a halogen and “n” is 1, 2, 3 or 4. When more than one halogen is present they may be the same or different and selected from the group consisting of F, Cl, Br and I, preferably F.

The term “C₁₋₆ haloalkylsulfinyl” denotes a haloalkyl radical attached to a sulfoxide group of the formula: —S(O)— wherein the haloalkyl radical has the same definition as described herein. Examples include, but not limited to, trifluoromethylsulfinyl, 2,2,2-trifluoroethylsulfinyl, 2,2-difluoroethylsulfinyl and the like.

The term “C₁₋₆ haloalkylsulfonyl” denotes a haloalkyl radical attached to a sulfone group of the formula: —S(O)₂— wherein haloalkyl has the same definition as described herein. Examples include, but not limited to, trifluoromethylsulfonyl, 2,2,2-trifluoroethyl sulfonyl, 2,2-difluoroethylsulfonyl and the like.

The term “C₁₋₆ haloalkylthio” denotes a haloalkyl radical directly attached to a sulfur wherein the haloalkyl has the same meaning as described herein. Examples include, but not limited to, trifluoromethylthio (i.e., CF₃S—, also referred to as trifluoromethylsulfanyl), 1,1-difluoroethylthio, 2,2,2-trifluoroethylthio and the like.

The term “halogen” or “halo” denotes to a fluoro, chloro, bromo or iodo group.

The term “heteroaryl” denotes an aromatic ring system that may be a single ring, two fused rings or three fused rings wherein at least one ring carbon is replaced with a heteroatom selected from, but not limited to, the group consisting of O, S and N wherein the N can be optionally substituted with H, C₁₋₄ acyl or C₁₋₄ alkyl. Examples of heteroaryl groups include, but not limited to, pyridyl, benzofuranyl, pyrazinyl, pyridazinyl, pyrimidinyl, triazinyl, quinoline, benzoxazole, benzothiazole, 1H-benzimidazole, isoquinoline, quinazoline, quinoxaline and the like. In some embodiments, the heteroaryl atom is O, S, NH, examples include, but not limited to, pyrrole, indole, and the like. Other examples include, but not limited to, those in TABLE 2, TABLE 3, and the like.

The term “heterocyclic” denotes a non-aromatic carbon ring (i.e., C₃₋₇ cycloalkyl or C₄₋₇ cycloalkenyl as defined herein) wherein one, two or three ring carbons are replaced by a heteroatom selected from, but not limited to, the group consisting of O, S, N, wherein the N can be optionally substituted with H, C₁₋₄ acyl or C₁₋₄ alkyl, and ring carbon atoms optionally substituted with oxo or a thiooxo thus forming a carbonyl or thiocarbonyl group. The heterocyclic group is a 3-, 4-, 5-, 6- or 7-membered containing ring. Examples of a heterocyclic group include but not limited to aziridin-1-yl, aziridin-2-yl, azetidin-1-yl, azetidin-2-yl, azetidin-3-yl, piperidin-1-yl, piperidin-4-yl, morpholin-4-yl, piperazin-1-yl, piperazin-4-yl, pyrrolidin-1-yl, pyrrolidin-3-yl, [1,3]-dioxolan-2-yl and the like.

The term “heterocycliccarboxamido” denotes a heterocyclic group, as defined herein, with a ring nitrogen where the ring nitrogen is bonded directly to the carbonyl forming an amide. Examples include, but not limited to,

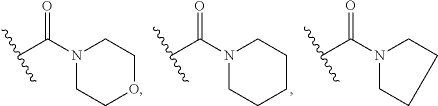

and the like.

The term “heterocyclicsulfonyl” denotes a heterocyclic group, as defined herein, with a ring nitrogen where the ring nitrogen is bonded directly to an —SO₂-group forming an sulfonamide. Examples include, but not limited to,

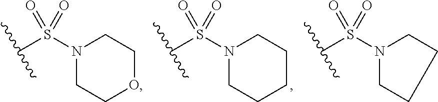

and the like.

The term “hydroxyl” refers to the group —OH.

The term “hydroxylamino” refers to the group —NHOH.

The term “nitro” refers to the group —NO₂.

The term “C₄₋₇ oxo-cycloalkyl” refers to a C₄₋₇ cycloalkyl, as defined herein, wherein one of the ring carbons is replaced with a carbonyl. Examples of C₄₋₇ oxo-cycloalkyl include, but are not limited to, 2-oxo-cyclobutyl, 3-oxo-cyclobutyl, 3-oxo-cyclopentyl, 4-oxo-cyclohexyl, and the like and represented by the following structures respectively:

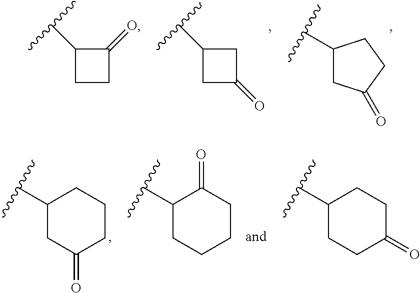

a perfluoroalkyl is an alkyl as defined herein wherein the alkyl is fully substituted with fluorine atoms and is therefore considered a subset of haloalkyl. Examples of perfluoroalkyls include CF₃, CF₂CF₃, CF₂CF₂CF₃, CF(CF₃)₂, CF₂CF₂CF₂CF₃, CF₂CF(CF₃)₂, CF(CF₃)CF₂CF₃ and the like.

The term “phenoxy” refers to the group C₆H₅O—.

The term “phenyl” refers to the group C₆H₅—.

The term“sulfonic acid” refers to the group —SO₃H.

The term “thiol” denotes the group —SH.

In some embodiments, the 5-HT_{2A} serotonin receptor inverse agonist is 1-[3-(4-bromo-2-methyl-2H-pyrazol-3-yl)-4-methoxy-phenyl]-3-(2,4-difluoro-phenyl)-urea or pharmaceutically acceptable salts, hydrates, polymorphs or solvates thereof. In some embodiments, 1-[3-(4-bromo-2-methyl-2H-pyrazol-3-yl)-4-methoxy-phenyl]-3-(2,4-difluoro-phenyl)-urea may also be known as nelotanserin or RVT-102 and these terms may be used interchangeably. In each of the embodiments described herein, the method may consist essentially of administering a therapeutically effective amount of nelotanserin or pharmaceutically acceptable salts, hydrates, polymorphs or solvates thereof. In each of the embodiments described herein, the method may consist of administering a therapeutically effective amount of nelotanserin or pharmaceutically acceptable salts, hydrates, polymorphs or solvates thereof. The present invention also encompasses the use of diastereomers as well as optical isomers, e.g. mixtures of enantiomers including racemic mixtures, as well as individual enantiomers and diastereomers, which arise as a consequence of structural asymmetry in certain compounds of the invention. Separation of the individual isomers or selective synthesis of the individual isomers is accomplished by application of various methods which are well known to practitioners in the art.

In some embodiments, the 5-HT_{2A} serotonin receptor inverse agonist is selected from those disclosed in U.S. Pat. No. 9,434,692, hereby incorporated in its entirety for any purpose. Particularly, the 5-HT_{2A} serotonin receptor inverse agonist can be to certain compounds as shown in Formula (Ia):

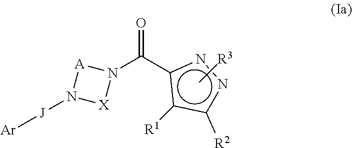

or a pharmaceutically acceptable salt, hydrate or solvate thereof; wherein R 1, R 2, R3, Ar, A, X and J have the same definitions as described herein, supra and infra.

In some embodiments, the compounds of the present invention are other than 1-(4-(1Hpyrazole-3-carbonyl)piperazin-1-yl)-2-(4-fluoro-1H-indol-3-yl)ethane-1,2-dione, represented by the formula below:

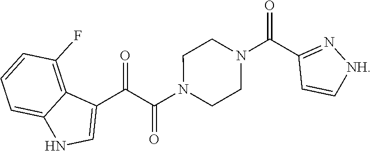 [SMILES: O=C(C(=O)N1CCN(C(=O)c2cc[nH]n2)CC1)c1c[nH]c2cccc(F)c12]

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the chemical groups represented by the variables (e.g., R1, R2, R3, Ar, A, X and J) contained within the generic chemical formulae described herein, for example, (Ia, le and le) are specifically embraced by the present invention just as if they were explicitly disclosed, to the extent that such combinations embrace compounds that result in stable compounds (i.e., compounds that can be isolated, characterized and tested for biological activity). In addition, all subcombinations of the chemical groups listed in the embodiments describing such variables, as well as all subcombinations of uses and medical indications described herein, are also specifically embraced by the present invention just as if each of such subcombination of chemical groups and subcombination of uses and medical indications were explicitly disclosed herein.

As used herein, “substituted” indicates that at least one hydrogen atom of the chemical group is replaced by a non-hydrogen substituent or group, the non-hydrogen substituent or group can be monovalent or divalent. When the substituent or group is divalent, then it is understood that this group is further substituted with another substituent or group. When a chemical group herein is “substituted” it may have up to the full valance of substitution; for example, a methyl group can be substituted by 1, 2, or 3 substituents, a methylene group can be substituted by 1 or 2 substituents, a phenyl group can be substituted by 1, 2, 3, 4, or 5 substituents, a naphthyl group can be substituted by 1, 2, 3, 4, 5, 6, or 7 substituents and the like.

Likewise, “substituted with one or more substituents” refers to the substitution of a group with one substituent up to the total number of substituents physically allowed by the group. Further, when a group is substituted with more than one group they can be identical or they can be different.

Compounds of the invention can also include tautomeric forms, such as keto-enol tautomers, and the like. Tautomeric forms can be in equilibrium or sterically locked into one form by appropriate substitution. It is understood that the various tautomeric forms are within the scope of the compounds of the present invention.

Compounds of the invention can also include all isotopes of atoms occurring in the intermediates and/or final compounds. Isotopes include those atoms having the same atomic number but different mass numbers. For example, isotopes of hydrogen include deuterium and tritium.

It is understood and appreciated that compounds of Formula (la) and formulae related therefrom may have one or more chiral centers, and therefore can exist as enantiomers and/or diastereomers. The invention is understood to extend to and embrace all such enantiomers, diastereomers and mixtures thereof, including but not limited to racemates. It is understood that compounds of Formula (Ia) and formulae used throughout this disclosure are intended to represent all individual enantiomers and mixtures thereof, unless stated or shown otherwise.

Some embodiments of the present invention pertain to compounds of Formula (lc):

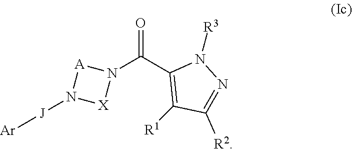

Some embodiments of the present invention pertain to compounds of Formula (le):

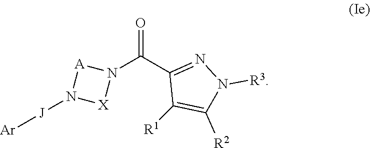

In some embodiments, each R1 and R2 is selected independently from the group consisting of H, C1-C6 alkyl, C1-C6 alkylaryl, aryl, C3-C7 cycloalkyl, C1-C6 haloalkyl, halogen, heteroaryl, and nitro.

In some embodiments, R1 and R2 is selected independently from the group consisting of H, methyl, ethyl, isopropyl, t-butyl, 2-methylphenyl, phenyl, cyclopropyl, trifluoromethyl, fluoro, chloro, bromo, iodo, furan-2-yl and nitro.

In some embodiments, R1 is H, halogen or C1-C6 alkylaryl; and R2 is H, C1-C6 alkyl, aryl, CrC7 cycloalkyl, C1-C6 haloalkyl, heteroaryl or nitro.

In some embodiments, R1 is H, fluoro, chloro, bromo, iodo or 2-methylphenyl and R2 is H, methyl, ethyl, isopropyl, t-butyl, phenyl, cyclopropyl, trifluoromethyl, furan-2-yl or nitro.

In some embodiments, R1 and R2 together with the carbon atoms to which they are bonded form a CrC7 carbocyclyl.

In some embodiments, R1 and R2 together with the carbon atoms to which they are bonded form a C5 carbocyclyl.

In some embodiments, R3 is selected from the group consisting of H, C1-C6 alkyl and aryl; and wherein aryl is optionally substituted with C1-C6 alkoxy.

In some embodiments, R3 is selected from the group consisting of H, C1-C6 alkyl and aryl; and wherein aryl is optionally substituted with methoxy.

In some embodiments, R3 is selected from the group consisting of H, methyl, ethyl, tbutyl, phenyl and 4-methoxyphenyl.

In some embodiments, A and X are each —CH2CHr, each optionally substituted with C1-C3 alkyl.

In some embodiments, A and X are each —CH2CHr, each optionally substituted with methyl.

In some embodiments, A and X are each independently-CH2CHi- or —CH(CH3)CHz-.

In some embodiments, J is —CH2CH2-optionally substituted with 1, 2, 3 or 4 substituents selected independently from the group consisting of C1-C3 alkyl, hydroxyl, oxo and ═NO—C1-C3 alkyl.

In some embodiments, J is —CH2CHr optionally substituted with 1, 2, 3 or 4 substituents selected independently from the group consisting of methyl, hydroxyl, oxo and ═NOCH3.

In some embodiments, J is —CH2CHz-, —C(═NOCH3)CHr, —C═OCHr, —CH(CH3)CHr, —C(CH3) 2CHr, or —CHOHCHz-.

In some embodiments, Ar is aryl or heteroaryl each optionally substituted with 1, 2, 3, 4 or 5 substituents selected independently from the group consisting of C1-C6 alkoxy, C1-C6 alkylsulfonyl, C1-C6 haloalkoxy, C1-C6 haloalkyl, halogen and heterocyclyl.

In some embodiments, Ar is aryl or heteroaryl each optionally substituted with 1, 2, 3, 4 or 5 substituents selected independently from the group consisting of methoxy, methanesulfonyl, trifluoromethoxy, trifluoromethyl, fluoro, chloro and pyrrolidin-1-yl.

In some embodiments, Ar is naphthyl, 2-methoxyphenyl, 4-methoxyphenyl, 4-methanesulfonylphenyl, 4-trifluoromethoxyphenyl, 4-trifluoromethylphenyl, 2-fluorophenyl, 3-fluorophenyl, 4-fluorophenyl, 2,4-difluorophenyl, 3,4-difluorophenyl, 2-chlorophenyl, 3-chlorophenyl, 4-chlorophenyl and 6-chloro-1,3-dihydro-indol-2-one.

Some embodiments of the present invention pertain to compounds of Formula (lc):

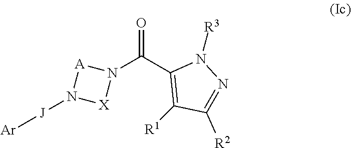

or a pharmaceutically acceptable salt, solvate or hydrate thereof;

wherein:

R1 is H, halogen or C1-C6 alkylaryl;

R2 is H, C1-C6 alkyl, aryl, CrC7 cycloalkyl, C1-C6 haloalkyl, heteroaryl, or nitro; or

R1 and R2 together with the carbon atoms to which they are bonded form a CrC7 carbocyclyl;

R3 is H, C1-C6 alkyl, aryl, or aryl substituted with C1-C6 alkoxy;

A and X are each —CH2CH2-, each optionally substituted with C1-C3 alkyl;

J is —CH2CHr optionally substituted with 1, 2, 3 or 4 substituents selected independently from the group consisting of C1-C3 alkyl, hydroxyl, oxo and ═NO—C1-C3 alkyl;

and

Ar is aryl or heteroaryl each optionally substituted with 1, 2, 3, 4 or 5 substituents selected independently from the group consisting of C1-C6 alkoxy, C1-C6 alkylsulfonyl, C1-C6 haloalkoxy, C1-C6 haloalkyl, halogen and heterocyclyl.

Some embodiments of the present invention pertain to compounds of Formula (lc):

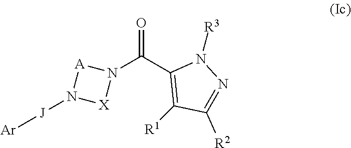

or a pharmaceutically acceptable salt, solvate or hydrate thereof;

wherein:

R1 is H, fluoro, chloro, bromo, iodo or 2-methylphenyl;

R2 is H, methyl, ethyl, isopropyl, t-butyl, phenyl, cyclopropyl, trifluoromethyl, furan-2-yl or nitro; or

R1 and R2 together with the carbon atoms to which they are bonded form a C5 carbocyclyl;

R3 is H, methyl, ethyl, t-butyl, phenyl or 4-methoxyphenyl;

A and X are each independently —CH2CHi- or —CH(CH3)CHi-;

J is —CH2CHi-, —C(═NOMe)CHr, —C═OCHr, —CH(CH3)CHr, —C(CH3) 2CHi-, or —CHOHCHr; and

Ar is naphthyl, 2-methoxyphenyl, 4-methoxyphenyl, 4-methanesulfonylphenyl, 4-trifluoromethoxyphenyl, 4-trifluoromethylphenyl, 2-fluorophenyl, 3-fluorophenyl, 4-fluorophenyl, 2,4-difluorophenyl, 3,4-difluorophenyl, 2-chlorophenyl, 3-chlorophenyl, 4-chlorophenyl and 6-chloro-1,3-dihydro-indol-2-one.

Some embodiments of the present invention pertain to compounds of Formula (le):

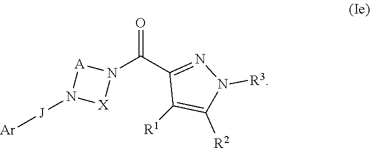

or a pharmaceutically acceptable salt, solvate or hydrate thereof;

wherein:

R1 is H, halogen or C1-C6 alkylaryl;

R2 is H, C1-C6 alkyl, aryl, C3-C7 cycloalkyl, C1-C6 haloalkyl, heteroaryl, or nitro; or

R1 and R2 together with the carbon atoms to which they are bonded form a CrC7 carbocyclyl;

R3 is H, C1-C6 alkyl, aryl, or aryl substituted with C1-C6 alkoxy;

A and X are each —CH2CHr, each optionally substituted with C1-C3 alkyl;

J is —CH2CHr optionally substituted with 1, 2, 3 or 4 substituents selected independently from the group consisting of C1-C3 alkyl, hydroxyl, oxo and ═NO—C1-C3 alkyl;

and

Ar is aryl or heteroaryl each optionally substituted with 1, 2, 3, 4 or 5 substituents selected independently from the group consisting of C1-C6 alkoxy, C1-C6 alkylsulfonyl, C1-C6 haloalkoxy, C1-C6 haloalkyl, halogen and heterocyclyl.

Some embodiments of the present invention pertain to compounds of Formula (le):

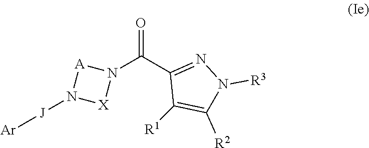

or a pharmaceutically acceptable salt, solvate or hydrate thereof;

wherein:

R1 is H, fluoro, chloro, bromo, iodo or 2-methylphenyl;

R2 is H, methyl, ethyl, isopropyl, t-butyl, phenyl, cyclopropyl, trifluoromethyl, furan-2-yl or nitro; or

R 1 and R 2 together with the carbon atoms to which they are bonded form a C5 carbocyclyl;

R3 is H, methyl, ethyl, t-butyl, phenyl or 4-methoxyphenyl;

A and X are each independently —CH2CHr or —CH(CH3)CHr;

J is —CH2CHr, —C(═NOMe)CHz-, —C═OCHr, —CH(CH3)CHr, —C(CH3)2CHr, or CHOHCHr;

and

Ar is naphthyl, 2-methoxyphenyl, 4-methoxyphenyl, 4-methanesulfonylphenyl, 4-trifluoromethoxyphenyl, 4-trifluoromethylphenyl, 2-fluorophenyl, 3-fluorophenyl, 4-fluorophenyl, 2,4-difluorophenyl, 3,4-difluorophenyl, 2-chlorophenyl, 3-chlorophenyl, 4-chlorophenyl and 6-chloro-1,3-dihydro-indol-2-one.

In some embodiments, where R1, R2 and R3 are all H; and A and X are both —CH2CHr; and J is (CO)z; then Ar is a moiety other than heteroaryl substituted with halogen.

Some embodiments of the present invention include every combination of one or more compounds selected from the following group shown in TABLE A.

| Cmpd | | |
|---|---|---|
| No. | Chemical Structure | Chemical Name |
| 1 | 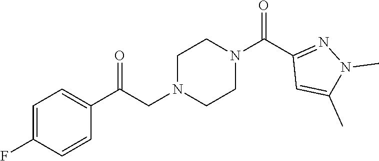 [SMILES: Cc1cc(C(=O)N2CCN(CC(=O)c3ccc(F)cc3)CC2)nn1C] | 2-[4-(1,5-Dimethyl-1H- pyrazole-3- carbonyl)piperazin-1-yl]- 1-(4-fluoro-phenyl)- ethanone |
| 2 | 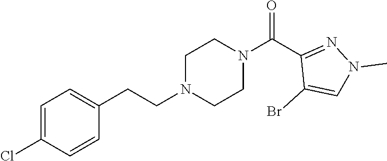 [SMILES: Cn1cc(Br)c(C(=O)N2CCN(CCc3ccc(Cl)cc3)CC2)n1] | (4-Bromo-1-methyl-1H- pyrazol-3-yl)-{4-[2-(4-chloro- phenyl)-ethyl]-piperazin-1- yl}-methanone |
| 3 | 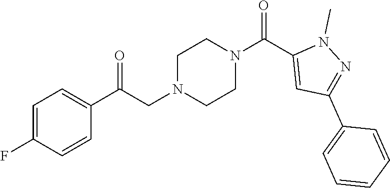 [SMILES: Cn1nc(-c2ccccc2)cc1C(=O)N1CCN(CC(=O)c2ccc(F)cc2)CC1] | 1-(4-Fluoro-phenyl)-2- [4-(2-methyl-5-phenyl- 2H-pyrazole-3-carbonyl) piperazin-1-yl]- ethanone |
| 4 | 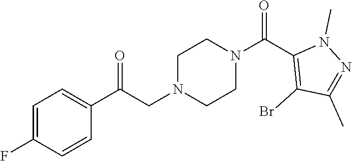 [SMILES: Cc1nn(C)c(C(=O)N2CCN(CC(=O)c3ccc(F)cc3)CC2)c1Br] | 2-[4-(4-Bromo-2,5-dimethyl- 2H-pyrazole-3-carbonyl)- piperazin-1-yl]-1- (4-fluoro-phenyl)- ethanone |
| 5 | 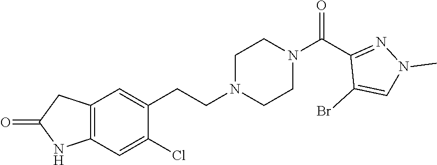 [SMILES: Cn1cc(Br)c(C(=O)N2CCN(CCc3cc4c(cc3Cl)NC(=O)C4)CC2)n1] | 5-{2-[4-(4-Bromo-1-methyl- 1H-pyrazole-3-carbonyl)- piperazin-1-yl]ethyl}-6- chloro-1,3-dihydro-indol-2- one |
| 6 | 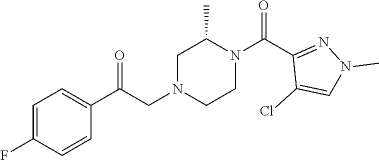 [SMILES: C[C@H]1CN(CC(=O)c2ccc(F)cc2)CCN1C(=O)c1nn(C)cc1Cl] | 2-[(S)-4-(4-Chloro-1-methyl- 1H-pyrazole-3-carbonyl)-3- methylpiperazin-1-yl]-1-(4- fluoro-phenyl)-ethanone |
| 7 | 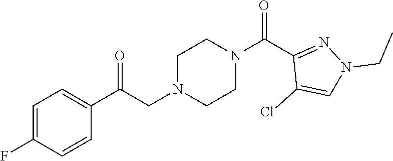 [SMILES: CCn1cc(Cl)c(C(=O)N2CCN(CC(=O)c3ccc(F)cc3)CC2)n1] | 2-[4-(4-Chloro-1-ethyl-1H- pyrazole-3-carbonyl)- piperazin-1-yl]-1- (4-fluoro-phenyl)- ethanone |
| 8 | 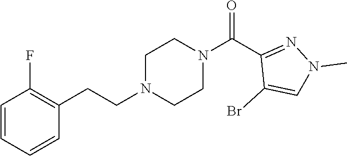 [SMILES: Cn1cc(Br)c(C(=O)N2CCN(CCc3ccccc3F)CC2)n1] | (4-Bromo-1-methyl-1H- pyrazol-3-yl)-{4-[2- (2-fluoro-phenyl)- ethyl]-piperazin-1- yl}-methanone |
| 9 | 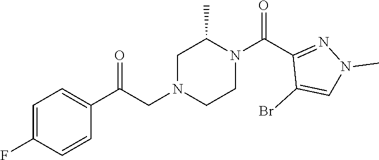 [SMILES: C[C@H]1CN(CC(=O)c2ccc(F)cc2)CCN1C(=O)c1nn(C)cc1Br] | 2-[(S)-4-(4-Bromo-1-methyl- 1H-pyrazole-3-carbonyl)-3- methyl-piperazin-1-yl]-1-(4- fluoro-phenyl)-ethanone |
| 10 | 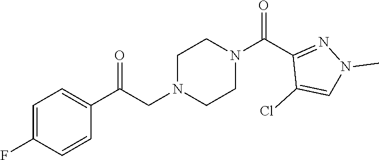 [SMILES: Cn1cc(Cl)c(C(=O)N2CCN(CC(=O)c3ccc(F)cc3)CC2)n1] | 2-[4-(4-Chloro-1-methyl- 1H-pyrazole-3-carbonyl)- piperazin-1-yl]-1-(4- fluoro-phenyl)-ethanone |
| 11 | 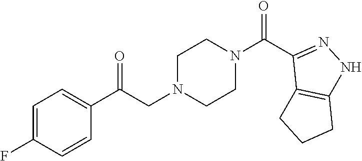 [SMILES: O=C(CN1CCN(C(=O)c2n[nH]c3c2CCC3)CC1)c1ccc(F)cc1] | 1-(4-Fluoro-phenyl)-2-[4- (1,4,5,6-tetrahydro- cyclopentapyrazole-3- carbonyl)-piperazin-1-yl]- ethanone |
| 12 | 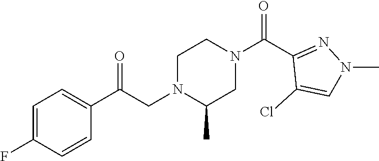 [SMILES: C[C@@H]1CN(C(=O)c2nn(C)cc2Cl)CCN1CC(=O)c1ccc(F)cc1] | 2-[(R)-4-(4-Chloro-1-methyl- 1H-pyrazole-3-carbonyl)-2- methyl-piperazin-1-yl]-1-(4- fluoro-phenyl)-ethanone |
| 13 | 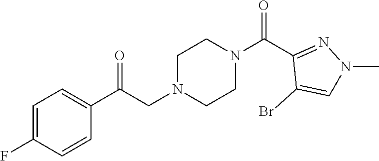 [SMILES: Cn1cc(Br)c(C(=O)N2CCN(CC(=O)c3ccc(F)cc3)CC2)n1] | 2-[4-(4-Bromo-1-methyl- 1H-pyrazole-3-carbonyl)- piperazin-1-yl]-1- (4-fluoro-phenyl)- ethanone |
| 14 | 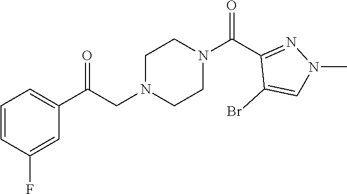 [SMILES: Cn1cc(Br)c(C(=O)N2CCN(CC(=O)c3cccc(F)c3)CC2)n1] | 2-[4-(4-Bromo-1-methyl- 1H-pyrazole-3-carbonyl)- piperazin-1-yl]-1- (3-fluoro-phenyl)- ethanone |
| 15 | 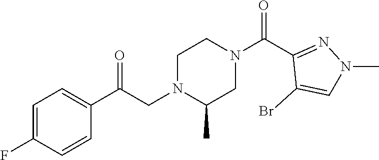 [SMILES: C[C@@H]1CN(C(=O)c2nn(C)cc2Br)CCN1CC(=O)c1ccc(F)cc1] | 2-[(R)-4-(4-Bromo-1- methyl-1H-pyrazole-3- carbonyl)-2-methyl- piperazin-1-yl]- 1-(4-fluoro-phenyl)- ethanone |
| 16 | 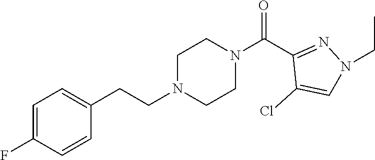 [SMILES: CCn1cc(Cl)c(C(=O)N2CCN(CCc3ccc(F)cc3)CC2)n1] | (4-Chloro-1-ethyl- 1H-pyrazol-3-yl)-{4-[2- (4-fluoro-phenyl)-ethyl]- piperazin-1-yl}- methanone |
| 17 | 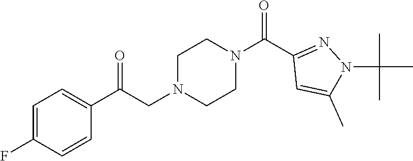 [SMILES: Cc1cc(C(=O)N2CCN(CC(=O)c3ccc(F)cc3)CC2)nn1C(C)(C)C] | 2-[4-(1-tert-Butyl-5-methyl- 1H-pyrazole-3-carbonyl)- piperazin-1-yl]-1- (4-fluoro-phenyl)- ethanone |
| 18 | 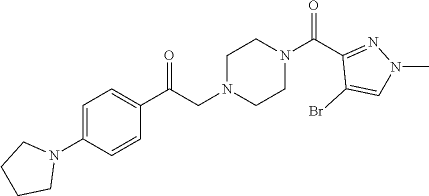 [SMILES: Cn1cc(Br)c(C(=O)N2CCN(CC(=O)c3ccc(N4CCCC4)cc3)CC2)n1] | 2-[4-(4-Bromo-1-methyl- 1H-pyrazole-3-carbonyl)- piperazin-1-yl]-1- (4-pyrrolidin-1-yl- phenyl)-ethanone |
| 19 | 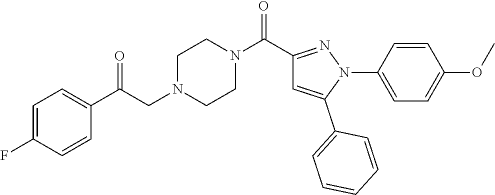 [SMILES: COc1ccc(-n2nc(C(=O)N3CCN(CC(=O)c4ccc(F)cc4)CC3)cc2-c2ccccc2)cc1] | 1-(4-Fluoro-phenyl)-2-{4-[1- (4-methoxy-phenyl)-5-phenyl- 1H-pyrazole-3-carbonyl]- piperazin-1-yl}-ethanone |
| 20 | 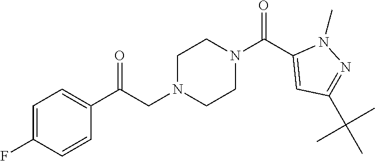 [SMILES: Cn1nc(C(C)(C)C)cc1C(=O)N1CCN(CC(=O)c2ccc(F)cc2)CC1] | 2-[4-(5-tert-Butyl-2-methyl- 2H-pyrazole-3-carbonyl)- piperazin-1-yl]-1- (4-fluoro-phenyl)-ethanone |
| 21 | 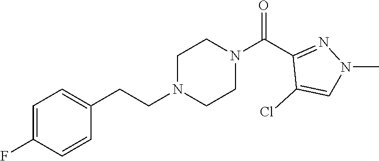 [SMILES: Cn1cc(Cl)c(C(=O)N2CCN(CCc3ccc(F)cc3)CC2)n1] | (4-Chloro-1-methyl- 1H-pyrazol-3-yl)- {4-[2-(4-fluoro-phenyl)- ethyl]-piperazin-1- yl}-methanone |
| 22 | 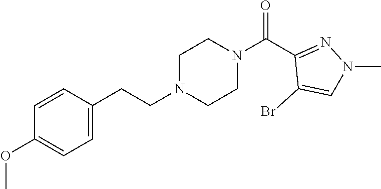 [SMILES: COc1ccc(CCN2CCN(C(=O)c3nn(C)cc3Br)CC2)cc1] | (4-Bromo-1-methyl- 1H-pyrazol-3-yl)- {4-[2-(4-methoxy-phenyl)- ethyl]-piperazin-1-yl}- methanone |
| 23 | 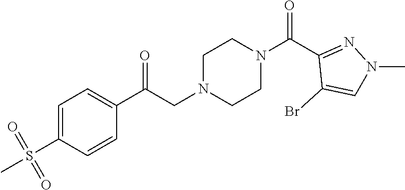 | 2-[4-(4-Bromo-1-methyl- 1H-pyrazole-3-carbonyl)- piperazin-1-yl]-1-(4- methanesulfonyl-phenyl)- ethanone |
| 24 | 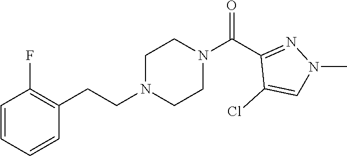 [SMILES: Cn1cc(Cl)c(C(=O)N2CCN(CCc3ccccc3F)CC2)n1] | (4-Chloro-1-methyl- 1H-pyrazol-3-yl)- {4-[2-(2-fluoro-phenyl)- ethyl]-piperazin-1-yl}- methanone |
| 25 | 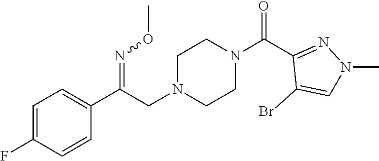 [SMILES: CON=C(CN1CCN(C(=O)c2nn(C)cc2Br)CC1)c1ccc(F)cc1] | 2-[4-(4-Bromo-1-methyl- 1H-pyrazole-3-carbonyl)- piperazin-1-yl]-1-(4- fluoro-phenyl)-ethanone O-methyl-oxime |
| 26 | 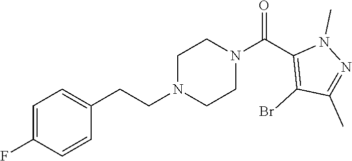 [SMILES: Cc1nn(C)c(C(=O)N2CCN(CCc3ccc(F)cc3)CC2)c1Br] | (4-Bromo-2,5-dimethyl- 2H-pyrazol-3-yl)-{4- [2-(4-fluoro- phenyl)-ethyl]- piperazin-1- yl}-methanone |
| 27 | 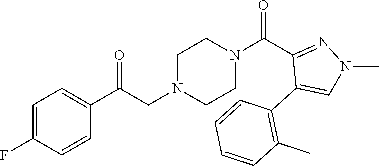 [SMILES: Cc1ccccc1-c1cn(C)nc1C(=O)N1CCN(CC(=O)c2ccc(F)cc2)CC1] | 1-(4-Fluoro-phenyl)-2- [4-(1-methyl-4-o-tolyl- 1H-pyrazole-3-carbonyl)- piperazin-1-yl]-ethanone |
| 28 | 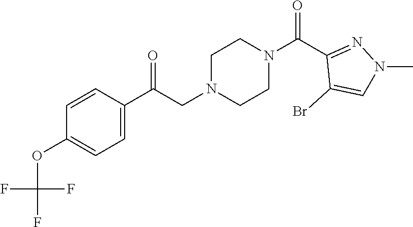 [SMILES: Cn1cc(Br)c(C(=O)N2CCN(CC(=O)c3ccc(OC(F)(F)F)cc3)CC2)n1] | 2-[4-(4-Bromo-1-methyl- 1H-pyrazole-3-carbonyl)- piperazin-1-yl]-1-(4- trifluoromethoxy-phenyl)- ethanone |
| 29 | 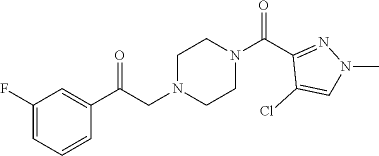 [SMILES: Cn1cc(Cl)c(C(=O)N2CCN(CC(=O)c3cccc(F)c3)CC2)n1] | 2-[4-(4-Chloro-1-methyl- 1H-pyrazole-3-carbonyl)- piperazin-1-yl]-1- (3-fluoro-phenyl)- ethanone |
| 30 | 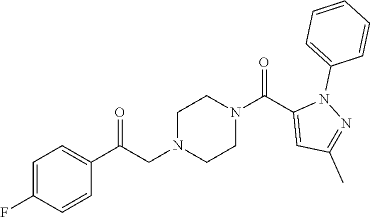 [SMILES: Cc1cc(C(=O)N2CCN(CC(=O)c3ccc(F)cc3)CC2)n(-c2ccccc2)n1] | 1-(4-Fluoro-phenyl)-2-[4-(5 methyl-2-phenyl-2H-pyrazole- 3-carbonyl)-piperazin-1-yl]- ethanone |
| 31 | 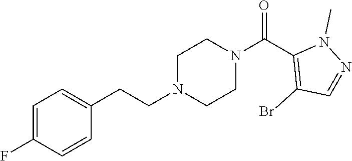 [SMILES: Cn1ncc(Br)c1C(=O)N1CCN(CCc2ccc(F)cc2)CC1] | (4-Bromo-2-methyl-2H- pyrazol-3-yl)-{4-[2-(4- fluoro-phenyl)- ethyl]-piperazin-1-yl}- methanone |
| 32 | 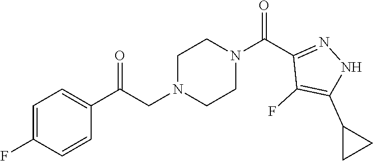 [SMILES: O=C(CN1CCN(C(=O)c2n[nH]c(C3CC3)c2F)CC1)c1ccc(F)cc1] | 2-(4-(5-Cyclopropyl-4- fluoro-1H-pyrazole-3- carbonyl)-piperazin-1-yl]- 1-(4-fluoro-phenyl)- ethanone |
| 33 | 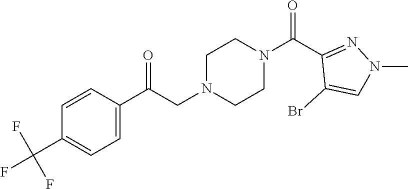 [SMILES: Cn1cc(Br)c(C(=O)N2CCN(CC(=O)c3ccc(C(F)(F)F)cc3)CC2)n1] | 2-[4-(4-Bromo-1-methyl- 1H-pyrazole-3-carbonyl)- piperazin-1-yl]-1-(4- trifluoromethyl-phenyl)- ethanone |
| 34 | 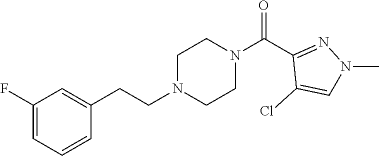 [SMILES: Cn1cc(Cl)c(C(=O)N2CCN(CCc3cccc(F)c3)CC2)n1] | (4-Chloro-1-methyl-1H- pyrazol-3-yl)-{4-[2-(3- fluoro-phenyl)-ethyl]- piperazin-1-yl}- methanone |
| 35 | 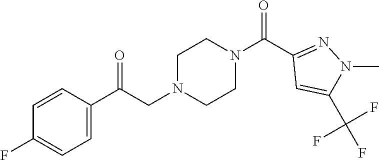 [SMILES: Cn1nc(C(=O)N2CCN(CC(=O)c3ccc(F)cc3)CC2)cc1C(F)(F)F] | 1-(4-Fluoro-phenyl)-2-[4-(1- methyl-5-trifluoromethyl- 1H-pyrazole-3-carbonyl)- piperazin-1-yl]-ethanone |
| 36 | 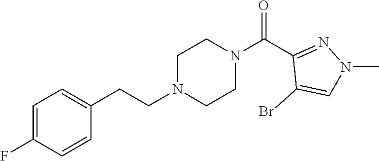 [SMILES: Cn1cc(Br)c(C(=O)N2CCN(CCc3ccc(F)cc3)CC2)n1] | (4-Bromo-1-methyl-1H- pyrazol-3-yl)-{4-[2-(4- fluoro-phenyl)-ethyl]- piperazin-1-yl}- methanone |
| 37 | 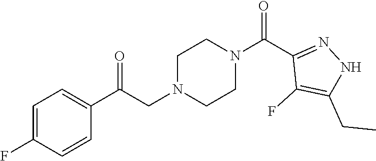 [SMILES: CCc1[nH]nc(C(=O)N2CCN(CC(=O)c3ccc(F)cc3)CC2)c1F] | 2-[4-(5-Ethyl-4-fluoro- 1H-pyrazole-3-carbonyl)- piperazin-1-yl]-1- (4-fluoro-phenyl)- ethanone |
| 38 | 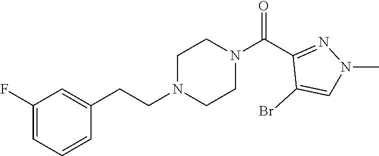 [SMILES: Cn1cc(Br)c(C(=O)N2CCN(CCc3cccc(F)c3)CC2)n1] | (4-Bromo-1-methyl-1H- pyrazol-3-yl)- {4-[2-(3-fluoro-phenyl)- ethyl]-piperazin-1- yl}-methanone |
| 39 | 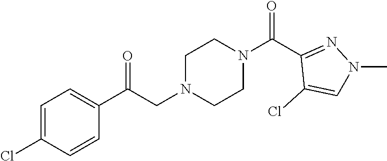 [SMILES: Cn1cc(Cl)c(C(=O)N2CCN(CC(=O)c3ccc(Cl)cc3)CC2)n1] | 2-[4-(4-Chloro-1-methyl- 1H-pyrazole-3-carbonyl)- piperazin-1-yl]-1- (4-chloro-phenyl)- ethanone |
| 40 | 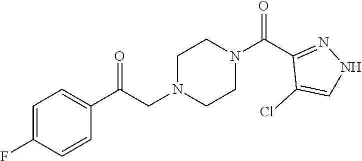 [SMILES: O=C(CN1CCN(C(=O)c2n[nH]cc2Cl)CC1)c1ccc(F)cc1] | 2-[4-(4-Chloro-1H-pyrazole-3- carbonyl)-piperazin-1-yl]-1- (4-fluoro-phenyl)-ethanone |
| 41 | 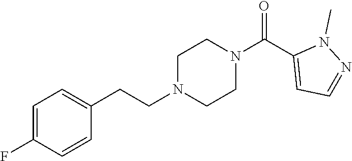 [SMILES: Cn1nccc1C(=O)N1CCN(CCc2ccc(F)cc2)CC1] | {4-[2-(4-Fluoro-phenyl)- ethyl]-piperazin-1-yl}-(2- methyl-2H-pyrazol-3-yl)- methanone |
| 42 | 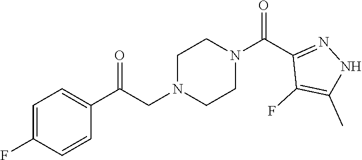 [SMILES: Cc1[nH]nc(C(=O)N2CCN(CC(=O)c3ccc(F)cc3)CC2)c1F] | 2-[4-(4-Fluoro-5-methyl- 1H-pyrazole-3-carbonyl)- piperazin-1-yl]-1-(4-fluoro- phenyl)-ethanone |
| 43 | 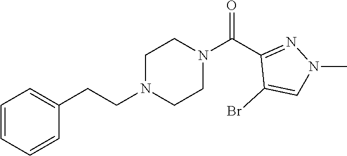 [SMILES: Cn1cc(Br)c(C(=O)N2CCN(CCc3ccccc3)CC2)n1] | (4-Bromo-1-methyl- 1H-pyrazol-3-yl)-(4- phenethyl-piperazin-1-yl)- methanone |
| 44 | 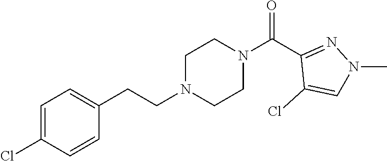 [SMILES: Cn1cc(Cl)c(C(=O)N2CCN(CCc3ccc(Cl)cc3)CC2)n1] | (4-Chloro-1-methyl-1H- pyrazol-3-yl)-{4-[2-(4- chloro-phenyl)-ethyl]- piperazin-1-yl}- methanone |
| 45 | 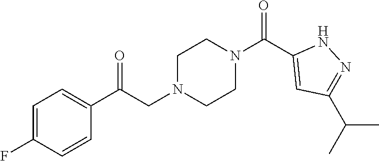 [SMILES: CC(C)c1cc(C(=O)N2CCN(CC(=O)c3ccc(F)cc3)CC2)[nH]n1] | 1-(4-Fluoro-phenyl)-2-[4-(5- isopropyl-2H-pyrazole-3- carbonyl)-piperazin-1-yl]- ethanone |
| 46 | 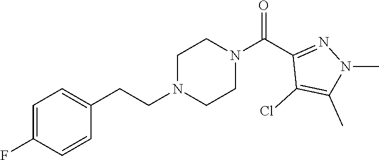 [SMILES: Cc1c(Cl)c(C(=O)N2CCN(CCc3ccc(F)cc3)CC2)nn1C] | (4-Chloro-1,5-dimethyl- 1H-pyrazol-3-yl)- {4-[2-(4-fluoro-phenyl)- ethyl]-piperazin-1- yl}-methanone |
| 47 | 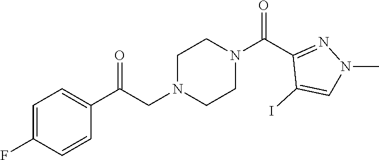 [SMILES: Cn1cc(I)c(C(=O)N2CCN(CC(=O)c3ccc(F)cc3)CC2)n1] | 1-(4-Fluoro-phenyl)-2- [4-(4-iodo-1-methyl-1H- pyrazole-3-carbonyl)- piperazin-1-yl]- ethanone |
| 48 | 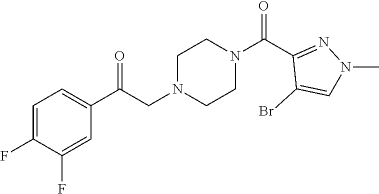 [SMILES: Cn1cc(Br)c(C(=O)N2CCN(CC(=O)c3ccc(F)c(F)c3)CC2)n1] | 2-[4-(4-Bromo-1- methyl-1H-pyrazole- 3-carbonyl)-piperazin- 1-yl]-1-(3,4-difluoro- phenyl)-ethanone |
| 49 | 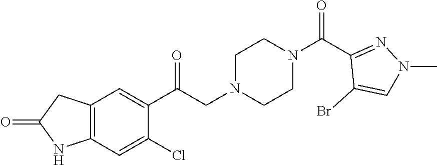 [SMILES: Cn1cc(Br)c(C(=O)N2CCN(CC(=O)c3cc4c(cc3Cl)NC(=O)C4)CC2)n1] | 5-{2-[4-(4-Bromo-1-methyl- 1H-pyrazole-3-carbonyl)- piperazin-1-yl]-acetyl}-6- chloro-1,3-dihydro-indol-2- one |
| 50 | 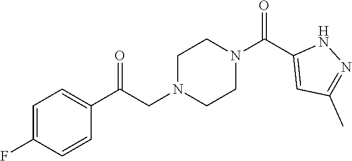 [SMILES: Cc1cc(C(=O)N2CCN(CC(=O)c3ccc(F)cc3)CC2)[nH]n1] | 1-(4-Fluoro-phenyl)-2-4-(5- methyl-2H-pyrazole-3- carbonyl)-piperazin-1-yl]- ethanone |
| 51 | 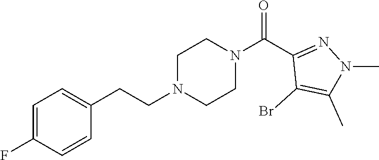 [SMILES: Cc1c(Br)c(C(=O)N2CCN(CCc3ccc(F)cc3)CC2)nn1C] | (4-Bromo-1,5-dimethyl-1H- pyrazol-3-yl)-{4-[2- (4-fluoro-phenyl)- ethyl]-piperazin-1- yl}-methanone |
| 52 | 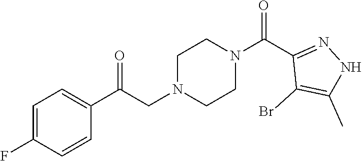 [SMILES: Cc1[nH]nc(C(=O)N2CCN(CC(=O)c3ccc(F)cc3)CC2)c1Br] | 2-[4-(4-Bromo-5-methyl- 1H-pyrazole-3-carbonyl)- piperazin-1-yl]-1- (4-fluoro-phenyl)- ethanone |
| 53 | 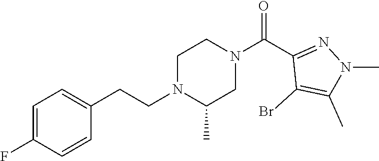 [SMILES: Cc1c(Br)c(C(=O)N2CCN(CCc3ccc(F)cc3)[C@@H](C)C2)nn1C] | (4-Bromo-1,5-dimethyl- 1H-pyrazol-3-yl)-{(S)-4-[2-(4- fluoro-phenyl)-ethyl]-3- methyl-piperazin-1-yl}- methanone |
| 54 | 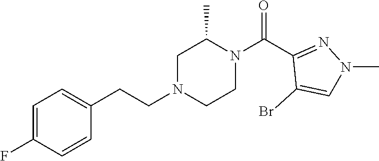 [SMILES: C[C@H]1CN(CCc2ccc(F)cc2)CCN1C(=O)c1nn(C)cc1Br] | (4-Bromo-1-methyl- 1H-pyrazol-3-yl)-{(S)-4-[2-(4- fluoro-phenyl)-ethyl]-2- methyl-piperazin-1-yl}- methanone |
| 55 | 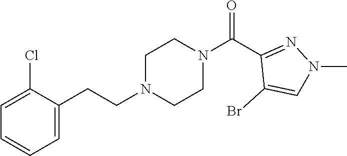 [SMILES: Cn1cc(Br)c(C(=O)N2CCN(CCc3ccccc3Cl)CC2)n1] | (4-Bromo-1-methyl- 1H-pyrazol-3-yl)- {4-[2-(2-chloro- phenyl)-ethyl]- piperazin-1- yl}-methanone |
| 56 | 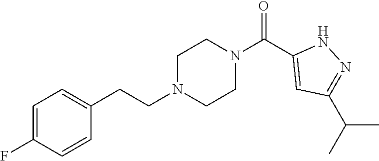 [SMILES: CC(C)c1cc(C(=O)N2CCN(CCc3ccc(F)cc3)CC2)[nH]n1] | {4-[2-(4-Fluoro-phenyl)- ethyl]-piperazin-1-yl]-(5- isopropyl-2H-pyrazol-3-yl)- methanone |
| 57 | 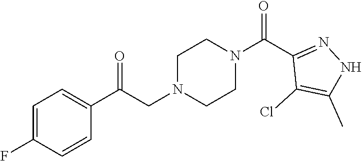 [SMILES: Cc1[nH]nc(C(=O)N2CCN(CC(=O)c3ccc(F)cc3)CC2)c1Cl] | 2-[4-(4-Chloro-5-methyl- 1H-pyrazole-3-carbonyl)- piperazin-1-yl]-(4-fluoro- phenyl)-ethanone |
| 58 | 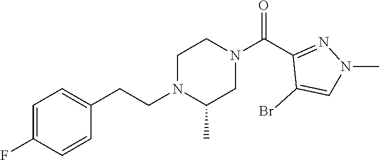 [SMILES: C[C@H]1CN(C(=O)c2nn(C)cc2Br)CCN1CCc1ccc(F)cc1] | (4-Bromo-1-methyl- 1H-pyrazol-3-yl)- {(S)-4-[2-(4-fluoro-phenyl)- ethyl]-3-methyl-piperazin- 1-yl}-methanone |
| 59 | 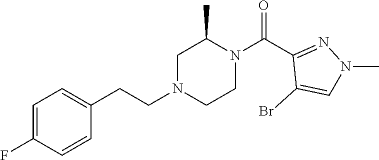 [SMILES: C[C@@H]1CN(CCc2ccc(F)cc2)CCN1C(=O)c1nn(C)cc1Br] | (4-Bromo-1-methyl-1H- pyrazol-3-yl)-{(R)-4-[2-(4- fluoro-phenyl)-ethyl]-2- methyl-piperazin-1-yl}- methanone |
| 60 | 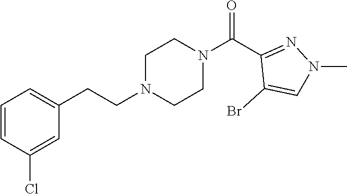 [SMILES: Cn1cc(Br)c(C(=O)N2CCN(CCc3cccc(Cl)c3)CC2)n1] | (4-Bromo-1-methyl- 1H-pyrazol-3-yl)- {4-[2-(3-chloro-phenyl)- ethyl]-piperazin-1-yl}- methanone |
| 61 | 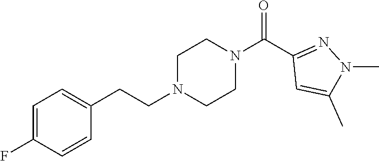 [SMILES: Cc1cc(C(=O)N2CCN(CCc3ccc(F)cc3)CC2)nn1C] | (1,5-Dimethyl-1H-pyrazol- 3-yl)-{4-[2-(4-fluorophenyl)- ethyl]-piperazin-1-yl}- methanone |
| 62 | 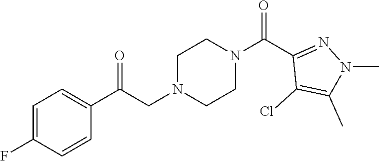 [SMILES: Cc1c(Cl)c(C(=O)N2CCN(CC(=O)c3ccc(F)cc3)CC2)nn1C] | 2-[4-(4-Chloro-1,5-dimethyl- 1H-pyrazole-3-carbonyl)- piperazin-1-yl]-1- (4-fluoro-phenyl)- ethanone |
| 63 | 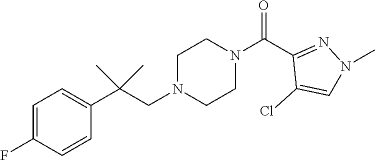 [SMILES: Cn1cc(Cl)c(C(=O)N2CCN(CC(C)(C)c3ccc(F)cc3)CC2)n1] | (4-Chloro-1-methyl- 1H-pyrazol-3-yl)-{4- [2-(4-fluoro-phenyl)-2- methyl-propyl]- piperazin-1-yl}- methanone |
| 64 | 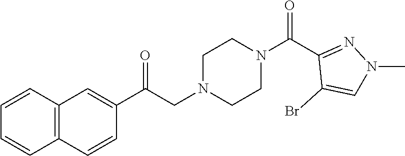 [SMILES: Cn1cc(Br)c(C(=O)N2CCN(CC(=O)c3ccc4ccccc4c3)CC2)n1] | 2-[4-(4-Bromo-1-methyl- 1H-pyrazole-3-carbonyl)- piperazin-1-yl]-1-naphthalen- 2-yl-ethanone |
| 65 | 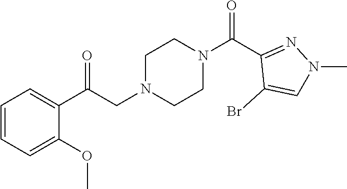 [SMILES: COc1ccccc1C(=O)CN1CCN(C(=O)c2nn(C)cc2Br)CC1] | 2-[4-(4-Bromo-1- methyl-1H-pyrazole-3- carbonyl)piperazin-1-yl]- 1-(2-methoxy-phenyl)- ethanone |
| 66 | 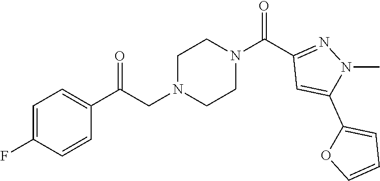 [SMILES: Cn1nc(C(=O)N2CCN(CC(=O)c3ccc(F)cc3)CC2)cc1-c1ccco1] | 1-(4-Fluoro-phenyl)-2-[4-(5- furan-2-yl-1-methyl- 1H-pyrazole-3-carbonyl) piperazin-1-yl]-ethanone |
| 67 | 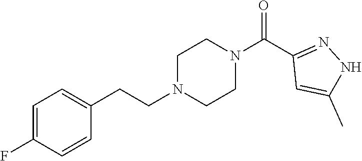 [SMILES: Cc1cc(C(=O)N2CCN(CCc3ccc(F)cc3)CC2)n[nH]1] | {4-[2-(4-Fluoro-phenyl)ethyl]- piperazin-1-yl}-(5-methyl-1H- pyrazol-3-yl)methanone |
| 68 | 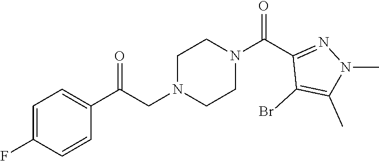 [SMILES: Cc1c(Br)c(C(=O)N2CCN(CC(=O)c3ccc(F)cc3)CC2)nn1C] | 2-[4-(4-Bromo-1,5-dimethyl- 1H-pyrazole-3-carbonyl)- piperazin-1-yl]-1-(4-fluoro- phenyl)ethanone |
| 69 | 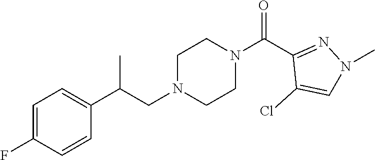 [SMILES: CC(CN1CCN(C(=O)c2nn(C)cc2Cl)CC1)c1ccc(F)cc1] | (4-Chloro-1-methyl-1H- pyrazol-3-yl)- {4-[2-(4-fluoro-phenyl)- propyl]-piperazin-1-yl}- methanone |
| 70 | 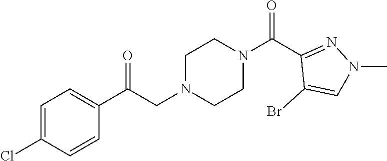 [SMILES: Cn1cc(Br)c(C(=O)N2CCN(CC(=O)c3ccc(Cl)cc3)CC2)n1] | 2-[4-(4-Bromo-1-methyl- 1H-pyrazole-3- carbonyl)piperazin-1-yl]- 1-(4-chloro-phenyl)- ethanone |
| 71 | 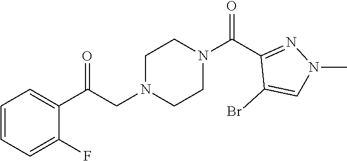 [SMILES: Cn1cc(Br)c(C(=O)N2CCN(CC(=O)c3ccccc3F)CC2)n1] | 2-[4-(4-Bromo-1-methyl- 1H-pyrazole-3- carbonyl)piperazin-1-yl]- 1-(2-fluoro-phenyl)- ethanone |
| 72 | 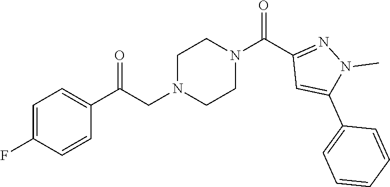 [SMILES: Cn1nc(C(=O)N2CCN(CC(=O)c3ccc(F)cc3)CC2)cc1-c1ccccc1] | 1-(4-Fluoro-phenyl)-2-[4- (1-methyl-5-pheny1- 1H-pyrazole-3- carbonyl)piperazin-1-yl]- ethanone |
| 73 | 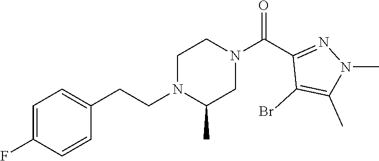 [SMILES: Cc1c(Br)c(C(=O)N2CCN(CCc3ccc(F)cc3)[C@H](C)C2)nn1C] | (4-Bromo-1,5-dimethyl-1H- pyrazol-3-yl)-{(R)-4-[2-(4- fluoro-phenyl)-ethyl]-3- methyl-piperazin-1-yl}- methanone |
| 74 | 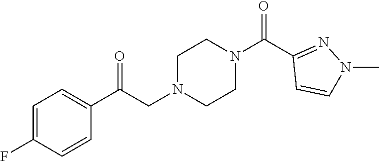 [SMILES: Cn1ccc(C(=O)N2CCN(CC(=O)c3ccc(F)cc3)CC2)n1] | 1-(4-Fluoro-phenyl)-2-[4- (1-methyl-1H-pyrazole-3- carbonyl)-piperazin-1-y1]- ethanone |
| 75 | 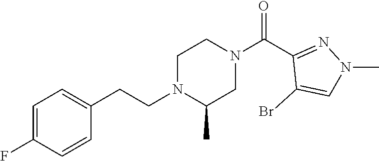 [SMILES: C[C@@H]1CN(C(=O)c2nn(C)cc2Br)CCN1CCc1ccc(F)cc1] | (4-Bromo-1-methyl-1H- pyrazol-3-yl)-{(R)-4-[2-(4- fluoro-phenyl)-ethyl]-3- methyl-piperazin-1-yl}- methanone |
| 76 | 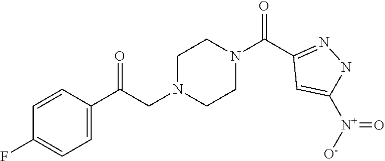 [SMILES: O=C(CN1CCN(C(=O)c2cc([N+](=O)[O-])[nH]n2)CC1)c1ccc(F)cc1] | 1-(4-Fluoro-phenyl)-2- [4-(5-nitro-1H-pyrazole- 3-carbonyl)-piperazin-1-yl]- ethanone |
| 77 | 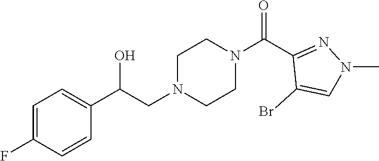 [SMILES: Cn1cc(Br)c(C(=O)N2CCN(CC(O)c3ccc(F)cc3)CC2)n1] | (4-Bromo-1-methyl- 1H-pyrazol-3-yl)- {4-[2-(4-fluoro- phenyl)-2-hydroxyethyl]- piperazin-1-yl}- methanone |
| 78 | 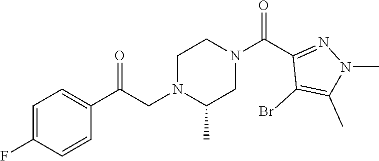 [SMILES: Cc1c(Br)c(C(=O)N2CCN(CC(=O)c3ccc(F)cc3)[C@@H](C)C2)nn1C] | 2-[(S)-4-(4-Bromo-1,5- dimethyl-1H-pyrazole-3- carbonyl)-2-methyl-piperazin- 1-yl]-1-(4-fluoro-phenyl)- ethanone |
| 79 | 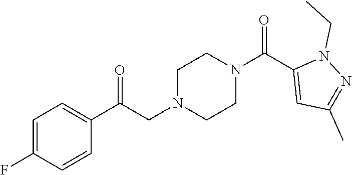 [SMILES: CCn1nc(C)cc1C(=O)N1CCN(CC(=O)c2ccc(F)cc2)CC1] | 2-[4-(2-Ethyl-5-methyl- 2H-pyrazole-3-carbonyl)- piperazin-1-yl]-1- (4-fluoro-phenyl)- ethanone |
| 80 | 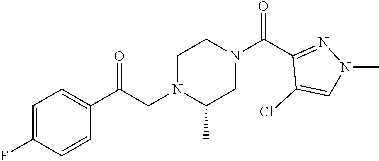 [SMILES: C[C@H]1CN(C(=O)c2nn(C)cc2Cl)CCN1CC(=O)c1ccc(F)cc1] | 2-[(S)-4-(4-Chloro-1-methyl- 1H-pyrazole-3-carbonyl)-2- methyl-piperazin-1-yl]-1-(4- fluoro-phenyl)-ethanone |
| 81 | 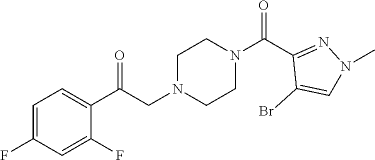 [SMILES: Cn1cc(Br)c(C(=O)N2CCN(CC(=O)c3ccc(F)cc3F)CC2)n1] | 2-[4-(4-Bromo-1-methyl- 1H-pyrazole-3-carbonyl) piperazin-1-yl]-1- (2,4-difluoro-phenyl)- ethanone |
| 82 | 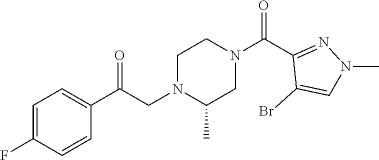 [SMILES: C[C@H]1CN(C(=O)c2nn(C)cc2Br)CCN1CC(=O)c1ccc(F)cc1] | 2-[(S)-4-(4-Bromo-1-methyl- 1H-pyrazole-3-carbonyl)-2- methyl-piperazin-1-yl]-1-(4- fluoro-phenyl)-ethanone |
| 83 | 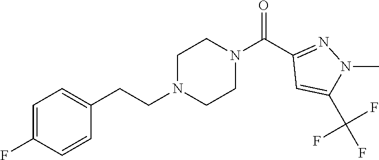 [SMILES: Cn1nc(C(=O)N2CCN(CCc3ccc(F)cc3)CC2)cc1C(F)(F)F] | {4-[2-(4-Fluoro-phenyl)ethyl]- piperazin-1-yl}-(1-methyl- 5-trifluoromethyl- 1H-pyrazol-3- yl)methanone |
| 84 | 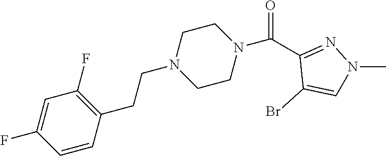 [SMILES: Cn1cc(Br)c(C(=O)N2CCN(CCc3ccc(F)cc3F)CC2)n1] | (4-Bromo-1-methyl-1H- pyrazol-3-yl)-{4-[2-(2,4- difluoro-phenyl)-ethyl]- piperazin-1-yl}-methanone |
| 85 | 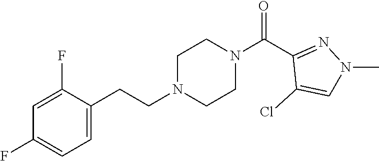 [SMILES: Cn1cc(Cl)c(C(=O)N2CCN(CCc3ccc(F)cc3F)CC2)n1] | (4-Chloro-1-methyl-1H- pyrazol-3-yl)-{4-[2-(2,4- difluoro-phenyl)-ethyl]- piperazin-1-yl}-methanone |

Additionally, individual compounds and chemical genera of the present invention, for example those compounds found in TABLE A including diastereomers and enantiomers thereof, encompass all pharmaceutically acceptable salts, solvates, and particularly hydrates, thereof.

The compounds of the Formula (Ia) of the present invention may be prepared according to relevant published literature procedures that are used by one skilled in the art. Exemplary reagents and procedures for these reactions appear hereinafter in the working Examples. Protection and deprotection may be carried out by procedures generally known in the art (see, for example, Greene, T. W. and Wuts, P. G. M., Protecting Groups in Organic Synthesis, 3rd Edition, 1999 [Wiley]; incorporated herein by reference in its entirety). It is understood that the present invention embraces each diastereomer, each enantiomer and mixtures thereof of each compound and generic formulae disclosed herein just as if they were each individually disclosed with the specific stereochemical designation for each chiral carbon. Separation of the individual isomers (such as, chiral HPLC, recrystallization of diastereomeric mixtures, and the like) or selective synthesis (such as, enantiomeric selective syntheses, and the like) of the individual isomers is accomplished by application of various methods which are well known to practitioners in the art.

The term “C1-C6 acyl” is intended to mean a C1-C6 alkyl radical attached to the carbon of a carbonyl group wherein the definition of alkyl has the same definition as described herein; some examples include, but are not limited to, acetyl, propionyl, n-butanoyl, iso-butanoyl, sec-butanoyl, t-butanoyl (i.e., pivaloyl), pentanoyl, and the like.

The term “C1-C6 acyloxy” is intended to mean an acyl radical attached to an oxygen atom wherein acyl has the same definition has described herein; some embodiments are when acyloxy is C1-C5 acyloxy, some embodiments are when acyloxy is C1-C4 acyloxy. Some examples include, but are not limited to, acetyloxy, propionyloxy, butanoyloxy, iso-butanoyloxy, sec-butanoyloxy, t-butanoyloxy, pentanoyloxy, hexanoyloxy, and the like.

The term “C2-C6 alkenyl” is intended to mean a radical containing 2 to 6 carbons wherein at least one carbon-carbon double bond is present, some embodiments are 2 to 5 carbons, some embodiments are 2 to 4 carbons, some embodiments are 2 to 3 carbons, and some embodiments have 2 carbons. Both E and Z isomers are embraced by the term “alkenyl.” Furthermore, the term “alkenyl” includes di- and tri-alkenyls. Accordingly, if more than one double bond is present then the bonds may be all E or all Z or a mixture thereof. Examples of an alkenyl include vinyl, allyl, 2-butenyl, 3-butenyl, 2-pentenyl, 3-pentenyl, 4-pentenyl, 2-hexenyl, 3-hexenyl, 4-hexenyl, 5-hexenyl, 2,4-hexadienyl and the like.

The term “C1-C6 alkoxy” is intended to mean a C1-C6 alkyl radical, as defined herein, attached directly to an oxygen atom, some embodiments are 1 to 5 carbons, some embodiments are 1 to 4 carbons, some embodiments are 1 to 3 carbons, and some embodiments are 1 or 2 carbons. Examples include methoxy, ethoxy, n-propoxy, iso-propoxy, n-butoxy, t-butoxy, iso-butoxy, sec-butoxy and the like.

The term “C1-C6 alkyl” is intended to mean a straight or branched carbon radical containing 1 to 6 carbons, some embodiments are 1 to 5 carbons, some embodiments are 1 to 4 carbons, some embodiments are 1 to 3 carbons, and some embodiments are 1 or 2 carbons. Examples of an alkyl include, but not limited to, methyl, ethyl, n-propyl, iso-propyl, n-butyl, sec-butyl, iso-butyl, t-butyl, pentyl, iso-pentyl, t-pentyl, neo-pentyl, 1-methylbutyl [i.e., CH(CH3)CH2CH2CH3], 2-methylbutyl [i.e., —CH2CH(CH3)CH2CH3], n-hexyl and the like.

The term “C1-C6 alkylaryl” is intended to mean a C1-C6 alkyl radical attached to an aromatic ring radical containing 6 to 10 ring carbons wherein the alkyl radical and the aryl radical have the same definitions as described herein. Examples include, but are not limited to tolyl and xylyl.

The term “C1-C6 alkylcarboxamido” or “C1-C6 alkylcarboxamide” is intended to mean a single C1-C6 alkyl group attached to either the carbon or the nitrogen of an amide group, wherein alkyl has the same definition as found herein. The C1-C6 alkylcarboxamido may be represented by the following:

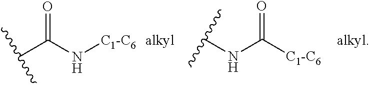

Examples include, but are not limited to, N-methylcarboxamide, N-ethylcarboxamide, N-n-propylcarboxamide, N-iso-propylcarboxamide, N-n-butylcarboxamide, N-sec-butylcarboxamide, N-iso-butylcarboxamide, N-t-butylcarboxamide and the like.

The term “C1-C6 alkylsulfinyl” is intended to mean a C1-C6 alkyl radical attached to the sulfur of a sulfoxide radical having the formula: —S(O)— wherein the alkyl radical has the same definition as described herein. Examples include, but are not limited to, methylsulfinyl, ethylsulfinyl, n-propylsulfinyl, iso-propylsulfinyl, n-butylsulfinyl, sec-butylsulfinyl, iso-butylsulfinyl, t-butylsulfinyl, and the like.

The term “C1-C6 alkylsulfonamide” is intended to mean the groups shown below:

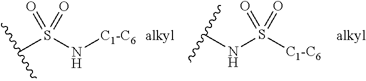

wherein C1-C6 alkyl has the same definition as described herein.

The term “C1-C6 alkylsulfonyl” is intended to mean a C1-C6 alkyl radical attached to the sulfur of a sulfone radical having the formula: —S(O)2- wherein the alkyl radical has the same definition as described herein. Examples include, but are not limited to, methyl sulfonyl, ethyl sulfonyl, n-propyl sulfonyl, iso-propylsulfonyl, n-butylsulfonyl, sec-butylsulfonyl, iso-butyl sulfonyl, t-butylsulfonyl, and the like.

The term “C1-C6 alkylthio” is intended to mean a C1-C6 alkyl radical attached to a sulfur atom (i.e., —S—) wherein the alkyl radical has the same definition as described herein. Examples include, but are not limited to, methylsulfanyl (i.e., CH3S—), ethylsulfanyl, n-propylsulfanyl, iso-propylsulfanyl, n-butylsulfanyl, sec-butylsulfanyl, iso-butylsulfanyl, t-butylsulfanyl, and the like.

The term “C1-C6 alkylureyl” is intended to mean the group of the formula: —NC(O)N— wherein one are both of the nitrogens are substituted with the same or different C1-C6 alkyl group wherein alkyl has the same definition as described herein. Examples of an alkylureyl include, but are not limited to, CH3NHC(O)NH—, NH2C(O)NCH3-, (CH3)2NC(O)NH—, (CH3)2NC(O)NCH3-, CH3 CH2NHC(O)NH—, CH3CH2NHC(O)NCH3-, and the like.

The term “C2-C6 alkynyl” is intended to mean a radical containing 2 to 6 carbons and at least one carbon-carbon triple bond, some embodiments have 2 to 4 carbons, some embodiments have 2 to 3 carbons, and some embodiments have 2 carbons. Examples of an alkynyl include, but are not limited to, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like. The term “alkynyl” includes di- and tri-ynes.

The term “amino” is intended to mean the group —NH2.

The term “C1-C6 alkylamino” is intended to mean one alkyl radical attached to a —NH-radical wherein the alkyl radical has the same meaning as described herein. Some examples include, but are not limited to, methylamino, ethylamino, n-propylamino, iso-propylamino, n-butylamino, sec-butylamino, iso-butylamino, t-butylamino, and the like. Some embodiments are “C1-C2 alkylamino.”

The term “aryl” is intended to mean an aromatic ring radical containing 6 to 10 ring carbons. Examples include phenyl and naphthyl.

The term “carbo-C1-C6-alkoxy” is intended to mean a C1-C6 alkyl ester of a carboxylic acid, wherein the alkyl group is as defined herein. Examples include, but are not limited to, carbomethoxy [—C(═O)OCH3], carboethoxy, carbopropoxy, carboisopropoxy, carbobutoxy, carbo-sec-butoxy, carbo-iso-butoxy, carbo-t-butoxy, carbo-n-pentoxy, carbo-iso-pentoxy, carbo-t-pentoxy, carbo-neo-pentoxy, carbo-n-hexyloxy, and the like.

The term “C3-C7 carbocyclyl” or “C3-C7 carbocyclic” is intended to mean a non-aromatic carbon ring (i.e., C3-C7 cycloalkyl or C4-C7 cycloalkenyl as defined herein).

The term “carboxamide” is intended to mean the group —CONH2.

The term “carboxy” or “carboxyl” is intended to mean the group —CO2H; also referred to as a carboxylic acid group.

The term “cyano” is intended to mean the group —CN.

The term “C4-C7 cycloalkenyl” is intended to mean a non-aromatic ring radical containing 4 to 7 ring carbons and at least one double bond; some embodiments contain 4 carbons; some embodiments contain 5 carbons; some embodiments contain 6 carbons; some embodiments contain 7 carbons. Examples include cyclobutenyl, cyclopentenyl, cyclohexenyl, cycloheptenyl and the like.

The term “C3-C7 cycloalkyl” is intended to mean a saturated ring radical containing 3 to 7 carbons; some embodiments contain 3 to 6 carbons; some embodiments contain 3 to 5 carbons; some embodiments contain 5 to 7 carbons; some embodiments contain 3 to 4 carbons. Examples include cyclopropyl, cyclobutyl, cyclopentyl, cyclohexyl, cycloheptyl and the like.

The term “C2-C6 dialkylamino” is intended to mean an amino substituted with two of the same or different C1-C3 alkyl radicals wherein alkyl radical has the same definition as described herein. Some examples include, but are not limited to, dimethylamino, methylethylamino, diethyl amino, methylpropylamino, methylisopropylamino, ethylpropylamino, ethylisopropylamino, dipropylamino, propylisopropylamino and the like. Some embodiments are “C2-C4 dialkylamino.”

The term “C2-C6 dialkylcarboxamido” or “C2-C6 dialkylcarboxamide” is intended to mean two alkyl radicals, that are the same or different, attached to an amide group, wherein alkyl has the same definition as described herein. A C2-C6 dialkylcarboxamido may be represented by the following groups:

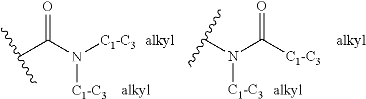

wherein C1-C3 has the same definition as described herein. Examples of a dialkylcarboxamide include, but are not limited to, N,N-dimethylcarboxamide, N-methyl-N-ethylcarboxamide, N,N-diethylcarboxamide, N-methyl-N-isopropylcarboxamide, and the like.

The term “C2-C6 dialkylsulfonamide” is intended to mean one of the following groups shown below:

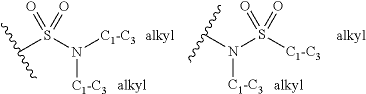

wherein C1-C3 has the same definition as described herein, for example but not limited to, methyl, ethyl, n-propyl, isopropyl, and the like.

The term “C1-C6 haloalkoxy” is intended to mean a C1-C6 haloalkyl, as defined herein, which is directly attached to an oxygen atom. Examples include, but are not limited to, difluoromethoxy, trifluoromethoxy, 2,2,2-trifluoroethoxy, pentafluoroethoxy and the like.

The term “C1-C6 haloalkyl” is intended to mean an C1-C6 alkyl group, defined herein, wherein the alkyl is substituted with one halogen up to fully substituted and a fully substituted C1-C6 haloalkyl can be represented by the formula CnL2n+1 wherein L is a halogen and “n” is 1, 2, 3, 4, 5 or 6; when more than one halogen is present then they may be the same or different and selected from the group consisting of F, Cl, Br and I, preferably F, some embodiments are 1 to 5 carbons, some embodiments are 1 to 4 carbons, some embodiments are 1 to 3 carbons, and some embodiments are 1 or 2 carbons. Examples of haloalkyl groups include, but are not limited to, fluoromethyl, difluoromethyl, trifluoromethyl, chlorodifluoromethyl, 2,2,2-trifluoroethyl, pentafluoroethyl and the like.

The term “C1-C6 haloalkylsulfinyl” is intended to mean a C1-C6 haloalkyl radical attached to the sulfur atom of a sulfoxide group having the formula: —S(O)— wherein the haloalkyl radical has the same definition as described herein. Examples include, but are not limited to, trifluoromethylsulfinyl, 2,2,2-trifluoroethylsulfinyl, 2,2-difluoroethylsulfinyl and the like.

The term “C1-C6 haloalkylsulfonyl” is intended to mean a C1-C6 haloalkyl radical attached to the sulfur atom of a sulfone group having the formula: —S(O)2- wherein haloalkyl has the same definition as described herein. Examples include, but are not limited to, trifluoromethylsulfonyl, 2,2,2-trifluoroethylsulfonyl, 2,2-difluoroethylsulfonyl and the like.

The term “C1-C6 haloalkylthio” is intended to mean a C1-C6 haloalkyl radical directly attached to a sulfur wherein the haloalkyl has the same meaning as described herein. Examples include, but are not limited to, trifluoromethylthio (i.e., CF3S—, also referred to as trifluoromethylsulfanyl), 1,1-difluoroethylthio, 2,2,2-trifluoroethylthio and the like.

The term “halogen” or “halo” is intended to mean to a fluoro, chloro, bromo or iodo group.

The term “heteroaryl” is intended to mean an aromatic ring system that may be a single ring, two fused rings or three fused rings wherein at least one ring carbon is replaced with a heteroatom selected from, for example, but not limited to, the group consisting of O, S and N wherein the N can be optionally substituted with H, C1-C4 acyl or C1-C4 alkyl. Examples of heteroaryl groups include, but are not limited to, pyridyl, benzofuranyl, pyrazinyl, pyridazinyl, pyrimidinyl, triazinyl, quinolinyl, benzoxazolyl, benzothiazolyl, 1H-benzimidazolyl, isoquinolinyl, quinazolinyl, quinoxalinyl and the like. In some embodiments, the heteroatom is selected from, for example, but not limited to, the group consisting of O, S and N, wherein N is substituted with H (i.e., NH), examples include, but are not limited to, pyrrolyl, indolyl, 1H-benzoimidazol-2-yl, and the like.

The term “C3-C7 heterocyclic” or “C3-C7 heterocyclyl” is intended to mean a non-aromatic carbon ring (i.e., C3-C7 cycloalkyl or C4-C7 cycloalkenyl as defined herein) wherein one, two or three ring carbons are replaced by a heteroatom selected from, for example, but not limited to, the group consisting of O, S, S(═O), S(═O)2, NH, wherein the N can be optionally substituted as described herein. In some embodiments, the nitrogen is optionally substituted with C1-C4 acyl or C1-C4 alkyl, and ring carbon atoms are optionally substituted with oxo or a thioxo thus forming a carbonyl or thiocarbonyl group. The heterocyclic group can be attached/bonded to any available ring atom, for example, ring carbon, ring nitrogen, and the like. The heterocyclic group is a 3-, 4-, 5-, 6- or 7-membered ring. Examples of a heterocyclic group include, but are not limited to, aziridin-1-yl, aziridin-2-yl, azetidin-1-yl, azetidin-2-yl, azetidin-3-yl, piperidin-1-yl, piperidin-2-yl, piperidin-3-yl, piperidin-4-yl, morpholin-2-yl, morpholin-3-yl, morpholin-4-yl, piperazin-1-yl, piperazin-2-yl, piperazin-3-yl, piperazin-4-yl, pyrrolidin-1-yl, pyrrolidin-2-yl, pyrrolidin-3-yl, [1,3]-dioxolan-2-yl, thiomorpholin-C4-yl, [1,4]oxazepan-4-yl, 1,1-dioxothiomorpholin-4-yl, azepan-1-yl, azepan-2-yl, azepan-3-yl, azepan-4-yl, tetrahydrofuran-2-yl, tetrahydrofuran-3-yl, and the like.

The term “hydroxyl” is intended to mean the group —OH.

The term “nitro” is intended to mean the group —NO2.

The term “oxo” is intended to mean the substituent ═O, accordingly, as a result, when a carbon is substituted by an “oxo” group the new group resulting from the carbon and oxo together is a carbonyl group.

The term “sulfonamide” is intended to mean the group —SO2NH2.

The term “thiol” is intended to mean the group —SH.

In some embodiments, the 5-HT_{2A} serotonin receptor inverse agonist is selected from 2-[(S)-4-(4-chloro-1-methyl-1-H-pyrazole-3-carbonyl)-3-methyl-piperazin-1-yl]-1-(4-fluoro-phenyl)-ethanone, 2-[4-(4-bromo-1-methyl-1-H-pyrazole-3-carbonyl)-piperazin-1-yl]-1-(4-fluoro-phenyl)-ethanone, 2-[4-(4-chloro-1-methyl-1-H-pyrazole-3-carbonyl)-piperazin-1-yl]-1-(4-fluoro-phenyl)-ethanone, or pharmaceutically acceptable salts, hydrates, polymorphs or solvates thereof.

The term “comprising” means “including, but not limited to.” The term “consisting essentially of” means the method or composition includes the steps or components specifically recited, and may also include those that do not materially affect the basic and novel characteristics of the present invention. The term “consisting of” means the method or composition includes only the steps or components specifically recited. It must be noted that, as used herein, and in the appended claims, the singular forms “a”, “an” and “the” include plural reference unless the context clearly dictates otherwise.

As used herein, the term “neuropsychiatric symptom” shall mean one or more psychiatric manifestation or behavioral disturbances associated with neurodegenerative diseases, such as, but not limited to depression, euphoria, delirium, delusions, flattening of affect, anxiety, dissociation, irritability, apathy, agitation, aggression, aberrant vocalizations, hallucinations, delusions, psychosis, wandering, sleep disturbances, sundowning, psychomotor retardation, cognitive impairment, disturbances of consciousness, behavioral changes, neurotic symptoms, mood disorders, Parkinsonism, nuchal rigidity, stiffness, personality change, neurological signs, somatic complaints, dementia, subcortical dementia, or disinhibition.

As used herein, the term “Parkinsonian symptom” shall mean one or more extrapyramidal motor or non-motor symptoms of Parkinson's disease including but not limited to tremor, bradykinesia, rigidity, postural instability or any combination thereof as well as non-motor symptoms including but not limited to mood disorders such as depression, anxiety and irritability, cognitive changes such as with focused attention and planning, slowing of thought, language and memory difficulties, personality changes, dementia, hallucinations and delusions, orthostatic hypotension, sleep disturbances or sleep disorders such as insomnia, excessive daytime sleepiness (EDS), rapid eye movement (REM) behavior disorder (RBD), vivid dreams, talking and moving during sleep, restless legs syndrome (RLS)/periodic leg movements disorder (PLMD), constipation and early satiety, pain, fatigue, vision problems, excessive sweating, increase in dandruff or oily skin, urinary urgency, frequency and incontinence, loss of sense of smell, sexual problems, weight loss or weight gain and impulsive control disorders such as binge eating, excessive shopping or gambling, or any combination thereof.

As used herein, the term “hallucination” means a sensory perception of the existence of something that is not real in the absence of external stimulus that has qualities of real perception. In some embodiments, hallucinations may be vivid, substantial, and are perceived to be located in external objective space. As used herein, hallucinations may occur in any sensory modality including, but not limited to visual, auditory, olfactory, gustatory, tactile, proprioceptive, equilibrioceptive, nociceptive, thermoceptive and chronoceptive. In some embodiments, the hallucinations are selected from visual hallucinations, auditory hallucinations, olfactory hallucinations, gustatory hallucinations, tactile hallucinations, proprioceptive hallucinations, equilibrioceptive hallucinations, nociceptive hallucinations, thermoceptive hallucinations, chronoceptive hallucinations and any combination thereof.

As used herein, the term “about” means plus or minus 10% of the numerical value of the number with which it is being used. Therefore, about 50% means in the range of 45%-55%.

“Optional” or “optionally” may be taken to mean that the subsequently described structure, event or circumstance may or may not occur, and that the described includes instances where the event occurs and instances where it does not.

“Administering” when used in conjunction with a therapeutic means to administer a therapeutic directly or indirectly into or onto a target tissue to administer a therapeutic to a patient whereby the therapeutic positively impacts the tissue to which it is targeted. “Administering” a composition may be accomplished by oral nasal, sublingual, buccal, transdermal, vaginal or rectal administration, injection, infusion, inhalation, absorption or by any method in combination with other known techniques. “Administering” may include the act of self-administration or administration by another person such as a health care provider.

The term “improves” is used to convey that the present invention changes the appearance, form, characteristics, structure, function and/or physical attributes of the tissue to which it is being provided, applied or administered. “Improves” may also refer to the overall physical state of an individual to whom an active agent has been administered. For example, the overall physical state of an individual may “improve” if one or more symptoms of the disease, condition or disorder are alleviated by administration of an active agent. The term “improve” as used herein may also refer a decrease in the severity, duration, frequency, or any combination thereof, in one or more symptoms of the disease.

As used herein, the term “therapeutic” means an agent utilized to treat, combat, ameliorate or prevent an unwanted disease, condition or disorder of a patient.

As used herein, the term “dopamine D₂ receptor” refers to the D₂ subtype of the dopamine receptor.

As used herein, the term “5HT_{2A} serotonin receptor” refers to the 2A subtype of the serotonin receptor.

As used herein, the term “agonist” refers to an agent that binds to a receptor and activates the receptor to produce a biological response.

As used herein, the term “inverse agonist” refers to an agent that binds to the same receptor as an agonist but induces a pharmacological response opposite to that agonist. In some embodiments, a prerequisite for an inverse agonist response is that the receptor must have a constitutive (also known as intrinsic or basal) level activity in the absence of any ligand. An agonist increases the activity of a receptor above its basal level, whereas an inverse agonist decreases the activity below the basal level.

The efficacy of a full agonist is by definition 100%, a neutral antagonist has 0% efficacy, and an inverse agonist has <0% (i.e., negative) efficacy.

As used herein, the term “baseline” is intended to represent an individual's status with respect to any of the diseases severity or progression scoring scales described herein prior to administration of the agents described herein.

In each of the embodiments disclosed herein, the compounds and methods may be utilized with or on an individual in need of such treatment, which may also be referred to as “in need thereof” As used herein, the phrase “in need thereof” means that the individual has been identified as having a need for the particular method or treatment and that the treatment has been given to the individual for that particular purpose. “In need thereof” as used herein also refers to a judgment made by a caregiver (e.g. physician, nurse, nurse practitioner, etc. in the case of humans; veterinarian in the case of animals, including non-human mammals) that an individual or animal requires or will benefit from prophylaxis and/or treatment. This judgment is made based on a variety of factors that are in the realm of a caregiver's expertise, but that includes the knowledge that the individual or animal is ill, or will be ill, as the result of a disease, condition or disorder that is treatable by the compounds of the invention. In general, “in need of prophylaxis” refers to the judgment made by the caregiver that the individual will become ill. In this context, the compounds of the invention are used in a protective or preventive manner. However, “in need of treatment” refers to the judgment of the caregiver that the individual is already ill; therefore, the compounds of the present invention are used to alleviate, inhibit or ameliorate the disease, condition or disorder.

As used herein, the term “patient” and “individual” or “individual” are interchangeable and may be taken to mean any living organism, which may be treated with compounds of the present invention. As such, the terms “patient” and “individual” may include, but are not limited to, any non-human mammal, primate or human. In some embodiments, the “patient” or “individual” is an adult, an elderly adult, child, infant, or fetus. In some embodiments, an elderly adult is an adult of about 50 years of age or older. In some embodiments, the “patient” or “individual” is a human. In some embodiments, the “patient” or “individual” is a mammal, such as mice, rats, other rodents, rabbits, dogs, cats, swine, cattle, sheep, horses, primates, or humans.

The term “therapeutically effective amount” as used herein refers to the amount of active compound or pharmaceutical agent that elicits the biological or medicinal response in a tissue, system, animal, individual or human that is being sought by a researcher, veterinarian, medical doctor or other clinician, which includes one or more of the following: (1) Preventing the disease; for example, preventing a disease, condition or disorder in an individual that may be predisposed to the disease, condition or disorder but does not yet experience or display the pathology or symptomatology of the disease, (2) Inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) Ameliorating the disease; for example, ameliorating a disease, condition or disorder in an individual that is experiencing or displaying the pathology or symptomatology of the disease, condition or disorder (i.e., reversing the pathology and/or symptomatology). In some embodiments, the therapeutically effective amount of nelotanserin or pharmaceutically acceptable salts, hydrates, polymorphs or solvates thereof is from about 0.0001 to about 1,000 mg. In some embodiments, the therapeutically effective amount of nelotanserin or pharmaceutically acceptable salts, hydrates, polymorphs or solvates thereof is from about 10 to about 160 mg. In some embodiments, the therapeutically effective amount of nelotanserin or pharmaceutically acceptable salts, hydrates, polymorphs or solvates thereof is about 10 mg. In some embodiments, the therapeutically effective amount of nelotanserin or pharmaceutically acceptable salts, hydrates, polymorphs or solvates thereof is about 20 mg. In some embodiments, the therapeutically effective amount of nelotanserin or pharmaceutically acceptable salts, hydrates, polymorphs or solvates thereof is about 40 mg. In some embodiments, the therapeutically effective amount of nelotanserin or pharmaceutically acceptable salts, hydrates, polymorphs or solvates thereof is about 80 mg. In some embodiments, the therapeutically effective amount of nelotanserin or pharmaceutically acceptable salts, hydrates, polymorphs or solvates thereof is about 160 mg. In some embodiments, the therapeutically effective amount of nelotanserin or pharmaceutically acceptable salts, hydrates, polymorphs or solvates thereof or a pharmaceutically acceptable salt, hydrate, polymorph, or solvate thereof is from about 0.001 mg to about 1,000 mg, about 0.001 mg to about 160 mg or about 10 to about 160 mg. In some embodiments, the therapeutically effective amount of nelotanserin or pharmaceutically acceptable salts, hydrates, polymorphs or solvates thereof is about 10 mg, about 20 mg, about 40 mg, about 80 mg or about 160 mg.

The term “treating” may be taken to mean prophylaxis of a specific disorder, disease or condition, alleviation of the symptoms associated with a specific disorder, disease or condition and/or prevention of the symptoms associated with a specific disorder, disease or condition. In some embodiments, the term refers to slowing the progression of the disorder, disease or condition or alleviating the symptoms associated with the specific disorder, disease or condition. In some embodiments, the term refers to alleviating the symptoms associated with the specific disorder, disease or condition. In some embodiments, the term refers to restoring function which was impaired or lost due to a specific disease, disorder or condition.

The term “pharmaceutical composition” shall mean a composition including at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan. A pharmaceutical composition may, for example, contain nelotanserin or pharmaceutically acceptable salts, hydrates, polymorphs or solvates thereof as the active ingredient.

“Pharmaceutically acceptable salts, hydrates, polymorphs or solvates” is meant to indicate those salts, hydrates, polymorphs or solvates which are, within the scope of sound medical judgment, suitable for use in contact with the tissues of a patient without undue toxicity, irritation, allergic response and the like, and are commensurate with a reasonable benefit/risk ratio. Pharmaceutically acceptable salts are well known in the art. For example, Berge et al. (1977) J. Pharm. Sciences, Vol 6. 1-19, describes pharmaceutically acceptable salts in detail. A pharmaceutical acceptable “salt” is any acid addition salt, preferably a pharmaceutically acceptable acid addition salt, including, but not limited to, halogenic acid salts such as hydrobromic, hydrochloric, hydrofluoric and hydroiodic acid salt; an inorganic acid salt such as, for example, nitric, perchloric, sulfuric and phosphoric acid salt; an organic acid salt such as, for example, sulfonic acid salts (methanesulfonic, trifluoromethan sulfonic, ethanesulfonic, benzenesulfonic or p-toluenesufonic, acetic, malic, fumaric, succinic, citric, benzoic gluconic, lactic, mandelic, mucic, pamoic, pantothenic, oxalic and maleic acid salts; and an amino acid salt such as aspartic or glutamic acid salt. The acid addition salt may be a mono- or di-acid addition salt, such as a di-hydrohalogic, di-sulfuric, di-phosphoric or di-organic acid salt. In all cases, the acid addition salt is used as an achiral reagent which is not selected on the basis of any expected or known preference for the interaction with or precipitation of a specific optical isomer of the products of this disclosure.

As used herein, the term “daily dose” refers to the amount of nelotanserin or pharmaceutically acceptable salts, hydrates, polymorphs or solvates thereof, per day that is administered or prescribed to a patient. This amount can be administered in multiple unit doses or in a single unit dose, at a single time during the day or at multiple times during the day. Multiple doses may be administered during the day, for example 2, 3 or 4, doses. In some embodiments, the dose is administered once daily in the morning, afternoon, evening, or once daily about 1 hour prior to the individual's bedtime. In some embodiments, the dose is administered about one to about four times per day, once daily in the morning, once daily about 1 hour prior to the individual's bedtime, or twice daily. In some embodiments, the dose is administered twice daily. In some embodiments, the daily dose of nelotanserin or pharmaceutically acceptable salts, hydrates, polymorphs or solvates thereof is from about 0.0001 to about 1,000 mg. In some embodiments, the daily dose of nelotanserin or pharmaceutically acceptable salts, hydrates, polymorphs or solvates thereof is from about 10 to about 160 mg. In some embodiments, the daily dose of nelotanserin or pharmaceutically acceptable salts, hydrates, polymorphs or solvates thereof is about 10 mg. In some embodiments, the daily dose of nelotanserin or pharmaceutically acceptable salts, hydrates, polymorphs or solvates thereof is about 20 mg. In some embodiments, the daily dose of nelotanserin or pharmaceutically acceptable salts, hydrates, polymorphs or solvates thereof is about 40 mg. In some embodiments, the daily dose of nelotanserin or pharmaceutically acceptable salts, hydrates, polymorphs or solvates thereof is about 80 mg. In some embodiments, the daily dose of nelotanserin or pharmaceutically acceptable salts, hydrates, polymorphs or solvates thereof is about 160 mg. In some embodiments, the daily dose of nelotanserin or pharmaceutically acceptable salts, hydrates, polymorphs or solvates thereof is from about 0.001 mg to about 1,000 mg, about 0.001 mg to about 160 mg or about 10 to about 160 mg. In some embodiments, the daily dose of nelotanserin or pharmaceutically acceptable salts, hydrates, polymorphs or solvates thereof is about 10 mg, about 20 mg, about 40 mg, about 80 mg or about 160 mg.

“Composition” shall mean a material comprising at least two compounds or two components; for example, and without limitation, a “Pharmaceutical Composition” is a Composition comprising a compound of the present invention and a pharmaceutically acceptable carrier.

Before the present compositions and methods are described, it is to be understood that this invention is not limited to the particular processes, compositions, or methodologies described, as these may vary. Moreover, the processes, compositions, and methodologies described in particular embodiments are interchangeable. Therefore, for example, a composition, dosages regimen, route of administration, and so on described in a particular embodiment may be used in any of the methods described in other particular embodiments. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods similar or equivalent to those described herein can be used in the practice or testing of embodiments of the present invention, the preferred methods are now described. All publications and references mentioned herein are incorporated by reference. Nothing herein is to be construed as an admission that the invention is not entitled to antedate such disclosure by virtue of prior invention.

The present invention is directed to the use of a 5-HT_{2A} serotonin receptor inverse agonist for the prophylaxis and/or treatment of the symptoms of dementia with Lewy bodies, Parkinson's disease, Parkinson's disease dementia, REM sleep behavior disorder, or a combination thereof in an individual comprising administering to said individual in need thereof a therapeutically effective amount of said 5-HT_{2A} serotonin receptor inverse agonist. In some embodiments, the symptoms of dementia with Lewy bodies, Parkinson's disease, or a combination thereof are Parkinsonian symptoms. In some embodiments, administering to said individual in need thereof a therapeutically effective amount of said 5-HT_{2A} serotonin receptor inverse agonist does not result in a worsening of neuropsychiatric symptoms such as, but not limited to hallucinations and delusions. In some embodiments, administering to said individual in need thereof a therapeutically effective amount of said 5-HT_{2A} serotonin receptor inverse agonist results in an improvement of neuropsychiatric symptoms such as, but not limited to hallucinations and delusions. In some embodiments, administering to said individual in need thereof a therapeutically effective amount of said 5-HT_{2A} serotonin receptor inverse agonist results in an improvement of neuropsychiatric symptoms and Parkinsonian symptoms. In some embodiments, administering to said individual in need thereof a therapeutically effective amount of said 5-HT_{2A} serotonin receptor inverse agonist does not result in QT interval prolongation. In some embodiments, administering to said individual in need thereof a therapeutically effective amount of said 5-HT_{2A} serotonin receptor inverse agonist results in an improvement of motor symptoms. In some embodiments, administering to said individual in need thereof a therapeutically effective amount of said 5-HT_{2A} serotonin receptor inverse agonist results in an improvement of Parkinsonian motor symptoms. In some embodiments, administering to said individual in need thereof a therapeutically effective amount of said 5-HT_{2A} serotonin receptor inverse agonist results in an improvement in sleep disturbances or the symptoms of a sleep disorder. In some embodiments, administering to said individual in need thereof a therapeutically effective amount of said 5-HT_{2A} serotonin receptor inverse agonist results in an improvement in sleep disturbances or the symptoms of a sleep disorder, an improvement of Parkinsonian symptoms, an improvement in neuropsychiatric symptoms or any combination thereof. In some embodiments, administering to said individual in need thereof a therapeutically effective amount of said 5-HT_{2A} serotonin receptor inverse agonist results in an improvement in sleep disturbances or the symptoms of a sleep disorder, an improvement of Parkinsonian symptoms, or a combination thereof. In some embodiments, administering to said individual in need thereof a therapeutically effective amount of said 5-HT_{2A} serotonin receptor inverse agonist results in an improvement of Parkinsonian symptoms, an improvement in neuropsychiatric symptoms or a combination thereof. In some embodiments, administering to said individual in need thereof a therapeutically effective amount of said 5-HT_{2A} serotonin receptor inverse agonist results in an improvement in sleep disturbances or the symptoms of a sleep disorder, an improvement in neuropsychiatric symptoms or a combination thereof. In some embodiments, administering to said individual in need thereof a therapeutically effective amount of said 5-HT_{2A} serotonin receptor inverse agonist results in an improvement in sleep disturbances or the symptoms of a sleep disorder, an improvement of Parkinsonian symptoms, an improvement in neuropsychiatric symptoms or any combination thereof, without causing a worsening of a of a sleep disturbance or sleep disorder, Parkinsonian symptom, or neuropsychiatric symptom or any combination thereof.

In some embodiments, the hallucinations are selected from visual hallucinations, auditory hallucinations, olfactory hallucinations, gustatory hallucinations, tactile hallucinations, proprioceptive hallucinations, equilibrioceptive hallucinations, nociceptive hallucinations, thermoceptive hallucinations, chronoceptive hallucinations and any combination thereof.

In some embodiments, the 5-HT_{2A} serotonin receptor inverse agonist is nelotanserin or a pharmaceutically acceptable salt, hydrate, polymorph, or solvate thereof. In some embodiments, the nelotanserin or a pharmaceutically acceptable salt, hydrate, polymorph, or solvate thereof is selected from the group consisting of Form I of 1-[3-(4-bromo-2-methyl-2H-pyrazol-3-yl)-4-methoxy-phenyl]-3-(2,4-difluoro-phenyl)-urea, Form II of 1-[3-(4-bromo-2-methyl-2H-pyrazol-3-yl)-4-methoxy-phenyl]-3-(2,4-difluoro-phenyl)-urea and a combination thereof. In some embodiments, a therapeutically effective amount of nelotanserin or a pharmaceutically acceptable salt, hydrate, polymorph, or solvate thereof is from about 10 mg to about 160 mg. In some embodiments, the therapeutically effective amount of nelotanserin or a pharmaceutically acceptable salt, hydrate, polymorph, or solvate thereof is about 10 mg, about 20 mg, about 40 mg, about 80 mg, or about 160 mg. In some embodiments, the therapeutically effective amount of nelotanserin or a pharmaceutically acceptable salt, hydrate, polymorph, or solvate thereof is about 10 mg, about 20 mg, about 40 mg, about 80 mg, or about 160 mg. In some embodiments, the therapeutically effective amount of nelotanserin or a pharmaceutically acceptable salt, hydrate, polymorph, or solvate thereof is about 10 mg. In some embodiments, the therapeutically effective amount of nelotanserin or a pharmaceutically acceptable salt, hydrate, polymorph, or solvate thereof is about 20 mg. In some embodiments, the therapeutically effective amount of nelotanserin or a pharmaceutically acceptable salt, hydrate, polymorph, or solvate thereof is about 40 mg. In some embodiments, the therapeutically effective amount of nelotanserin or a pharmaceutically acceptable salt, hydrate, polymorph, or solvate thereof is about 80 mg. In some embodiments, the therapeutically effective amount of nelotanserin or a pharmaceutically acceptable salt, hydrate, polymorph, or solvate thereof is about 160 mg.

In some embodiments, the therapeutically effective amount of nelotanserin or a pharmaceutically acceptable salt, hydrate, polymorph, or solvate thereof is administered once a day, twice a day, three times a day, or four times a day. In some embodiments, the nelotanserin or a pharmaceutically acceptable salt, hydrate, polymorph, or solvate thereof is in a pharmaceutical composition configured for immediate release, for extended release, for delayed release, or any combination thereof. In some embodiments, nelotanserin or a pharmaceutically acceptable salt, hydrate, polymorph, or solvate thereof is in a pharmaceutical composition, and wherein the pharmaceutical composition is formulated for oral administration. In some embodiments, the therapeutically effective amount of nelotanserin or a pharmaceutically acceptable salt, hydrate, polymorph, or solvate thereof is administered about one to about four times per day, once daily in the morning, once daily about 1 hour prior to the individual's bedtime, or twice daily.

In some embodiments, the individual is a human. In some embodiments, the human is an adult with a diagnosis of a condition selected from frontotemporal dementia, progressive supranuclear palsy, Lewy body dementia, probable dementia with Lewy bodies, dementia with Lewy bodies, Parkinson's disease dementia, Parkinson's disease, multiple system atrophy, Alzheimer's disease, vascular dementia, dementia, mild cognitive impairment, Parkinson's disease psychosis, Alzheimer's disease psychosis, a sleep disturbance, insomnia, delusions, agitation, Alzheimer's agitation, aggression, REM sleep behavior disorder, schizophrenia, and any combination thereof. In some embodiments, the human has a concurrent diagnosis of hallucinations, delusions, or a combination thereof, and a condition selected from Lewy body dementia, probable dementia with Lewy bodies, dementia with Lewy bodies, Parkinson's disease dementia, Parkinson's disease, multiple system atrophy, Alzheimer's disease, vascular dementia, dementia, mild cognitive impairment, Parkinson's disease psychosis, Alzheimer's disease psychosis, a sleep disturbance, insomnia, delusions, agitation, Alzheimer's agitation, aggression, REM sleep behavior disorder, schizophrenia, and any combination thereof. In some embodiments, the human has a concurrent diagnosis of hallucinations, delusions or a combination thereof, and a condition selected from frontotemporal dementia, progressive supranuclear palsy, Lewy body dementia, probable dementia with Lewy bodies, dementia with Lewy bodies, Parkinson's disease dementia, Parkinson's disease, multiple system atrophy, Alzheimer's disease, vascular dementia, dementia, mild cognitive impairment, Parkinson's disease psychosis, Alzheimer's disease psychosis, a sleep disturbance, insomnia, delusions, agitation, Alzheimer's agitation, aggression, REM sleep behavior disorder, schizophrenia, and any combination thereof. In some embodiments, the human has a diagnosis of probable dementia with Lewy bodies. In some embodiments, the diagnosis of probable dementia with Lewy Bodies is defined by the presence of dementia and at least one of: at least two Core Criteria selected from visual hallucinations, cognitive fluctuations, and Parkinsonism, and any combination thereof; and one Core Criteria selected from hallucinations, cognitive fluctuations, and Parkinsonism, and any combination thereof; and at least one Suggestive Criteria selected from REM sleep behavior disorder, severe neuroleptic sensitivity, low dopamine transporter uptake on DaT SPECT Imaging Scan; and any combination thereof. In some embodiments, the human has a diagnosis of dementia with Lewy Bodies. In some embodiments, the human has a diagnosis of Parkinson's disease. In some embodiments, the human has a diagnosis of Parkinson's disease dementia. In some embodiments, the human has a Mini Mental State Examination score of greater than, or equal to, about 18. In some embodiments, the human is an adult with a diagnosis of hallucinations, delusions or a combination thereof, associated with dementia with Lewy bodies. In some embodiments, the human is an adult with a diagnosis of hallucinations, delusions or a combination thereof, associated with Parkinson's disease. In some embodiments, the human is an adult with a diagnosis of hallucinations, delusions or a combination thereof, associated with Parkinson's disease dementia. In some embodiments, the individual has a score of zero or greater on (Scale for Assessing Positive symptoms) SAPS-H prior to administration of a therapeutically effective amount of nelotanserin or a pharmaceutically acceptable salt, hydrate, polymorph, or solvate thereof. In some embodiments, the individual has a score of one or greater on (Scale for Assessing Positive symptoms) SAPS-H prior to administration of a therapeutically effective amount of nelotanserin or a pharmaceutically acceptable salt, hydrate, polymorph, or solvate thereof. In some embodiments, the individual has a score of two or greater on (Scale for Assessing Positive symptoms) SAPS-H prior to administration of a therapeutically effective amount of nelotanserin or a pharmaceutically acceptable salt, hydrate, polymorph, or solvate thereof. In some embodiments, the individual has a score of three or greater on (Scale for Assessing Positive symptoms) SAPS-H prior to administration of a therapeutically effective amount of nelotanserin or a pharmaceutically acceptable salt, hydrate, polymorph, or solvate thereof. In some embodiments, the individual has a score of four or greater on (Scale for Assessing Positive symptoms) SAPS-H prior to administration of a therapeutically effective amount of nelotanserin or a pharmaceutically acceptable salt, hydrate, polymorph, or solvate thereof.

In some embodiments, administration of nelotanserin or a pharmaceutically acceptable salt, hydrate, polymorph, or solvate thereof results in an improvement in the individuals' Parkinson's disease-adapted scale for assessment of positive symptoms (SAPS-PD) as the primary endpoint. In some embodiments, administration of nelotanserin or a pharmaceutically acceptable salt, hydrate, polymorph, or solvate thereof results in an improvement in the individuals' sleep with the Parkinson's disease sleep scale (PDSS-2), to assess dyskinesia with the Unified dyskinesia rating scale (UDysRs), or a combination thereof. In some embodiments, administration of nelotanserin or a pharmaceutically acceptable salt, hydrate, polymorph, or solvate thereof results in an improvement in the individuals' parkinsonism with the Unified Parkinson's disease rating scale (UPDRS), to assess hallucinations and delusions with the Clinical global impression-severity scale (CGI-S) and the clinical global impression-improvement scale (CGI-I), which are used to assess the effect of nelotanserin versus placebo on daytime sleepiness as measured by the Epworth Sleepiness Scale (ESS), to assess the effects of nelotanserin versus placebo on proportion of subjects who lack insight into their most sever hallucinations and/or delusions, to assess the effect of nelotanserin versus placebo on hallucinations and delusions as measured by the scale for assessment of positive symptoms (SAPS), or a combination thereof. In some embodiments, administration of nelotanserin or a pharmaceutically acceptable salt, hydrate, polymorph, or solvate thereof results in an improvement in the individuals' overall quality of life with the Parkinson's disease questionnaire (PDQ8). In some embodiments, key entry criteria may be an age of greater than or equal to 40 years, idiopathic Parkinson's disease with a minimum duration of about 1 year, psychosis symptoms developed after the diagnosis of Parkinson's disease, active symptoms 4 weeks prior to screening, screening neuropsychiatric inventory (NPI) hallucinations score of greater than or equal to 4 or delusions score of greater than or equal to 4 or a total combined hallucinations and delusions score of greater than or equal to 6, a SAPS hallucinations or delusions global item (H7 or D13) score of greater than or equal to 3 and a score of greater than 3 on at least one other non-global item using the modified 9-item SAPS hallucinations and delusions domains at randomization, a mini-mental state examination (MMSE) score of greater than or equal to 21, a stable does of anti-Parkinson's medication, or a combination thereof. In some embodiments, the clinical trial may allow for stable quetiapine usage up to 100 mg per day which may be capped at 20% of the study population.

In some embodiments, administration of nelotanserin or a pharmaceutically acceptable salt, hydrate, polymorph, or solvate thereof results in an improvement in the individuals' Parkinson's disease-adapted scale for assessment of positive symptoms (SAPS-PD) as the primary endpoint. In some embodiments, the SAPS-PD is improved. In further embodiments, administration of nelotanserin or a pharmaceutically acceptable salt, hydrate, polymorph, or solvate thereof results in an improvement in the individuals' Parkinson's disease sleep scale (PDSS-2), the Unified dyskinesia rating scale (UDysRs), or a combination thereof. In some embodiments, administration of nelotanserin or a pharmaceutically acceptable salt, hydrate, polymorph, or solvate thereof results in an improvement in the individuals' Unified Parkinson's disease rating scale (UPDRS), Clinical global impression-severity scale (CGI-S) and the clinical global impression-improvement scale (CGI-I), the Epworth Sleepiness Scale (ESS), the effects of nelotanserin versus placebo on proportion of subjects who lack insight into their most severe hallucinations and/or delusions, the effect of nelotanserin versus placebo on hallucinations and delusions as measured by the scale for assessment of positive symptoms (SAPS), or a combination thereof. In some embodiments, administration of nelotanserin or a pharmaceutically acceptable salt, hydrate, polymorph, or solvate thereof results in an improvement in the individuals' overall quality of life with the Parkinson's disease questionnaire (PDQ8).

In some embodiments, the individual is concurrently receiving a therapeutically effective amount of at least one additional therapeutic agent selected from the group consisting of aripiprazole, asenapine, brexpiprazole, cariprazine, clozapine, iloperidone, lurasidone, olanzapine, paliperidone, risperidone, ziprasidone, pimavanserin, melatonin, quetiapine, clonazepam, levodopa, carbidopa, an antiparkinsonian drug, an acetylcholinesterase inhibitor, NMDA receptor antagonist, and a combination thereof. In some embodiments, the antiparkinsonian drug is selected from an MAO-B inhibitor, a COMT inhibitor, a dopamine agonist or any combination thereof. In some embodiments, the acetylcholinesterase inhibitor is selected from the group consisting of donepezil, rivastigmine, galantamine, and pharmaceutically acceptable salts, hydrates, polymorphs, or solvates thereof. In some embodiments, the acetylcholinesterase inhibitor is donepezil or a pharmaceutically acceptable salt, hydrate, polymorph, or solvate thereof. In some embodiments, the acetylcholinesterase inhibitor is rivastigmine or a pharmaceutically acceptable salt, hydrate, polymorph, or solvate thereof. In some embodiments, the acetylcholinesterase inhibitor is galantamine or a pharmaceutically acceptable salt, hydrate, polymorph, or solvate thereof. In some embodiments, NMDA receptor antagonist is selected from the group consisting of memantine, amantadine, ketamine, and pharmaceutically acceptable salts, hydrates, polymorphs, or solvates thereof. In some embodiments, the NMDA receptor antagonist is memantine or a pharmaceutically acceptable salt, hydrate, polymorph, or solvate thereof. In some embodiments, the NMDA receptor antagonist is amantadine or a pharmaceutically acceptable salt, hydrate, polymorph, or solvate thereof.

In some embodiments, administration of a therapeutically effective amount of nelotanserin or a pharmaceutically acceptable salt, hydrate, polymorph, or solvate thereof results in treatment, and/or prophylaxis of the Parkinsonian symptoms of dementia with Lewy bodies, Parkinson's disease, Parkinson's disease dementia, or a combination thereof.

In some embodiments, treatment, and/or prophylaxis of the Parkinsonian symptoms of dementia with Lewy bodies, Parkinson's disease, Parkinson's disease dementia, or a combination thereof results in an improvement in the individuals Unified Parkinson's Disease Rate Scale (UPDRS) compared to baseline. The UPDRS is used to measure worsening of a disease state and an improvement in an individual's UPDRS is indicative of an improvement in the disease state and efficacy of the treatment and in particular in an improvement in Parkinsonian symptoms in an individual. Parkinsonian symptoms include but are not limited to tremor, bradykinesia, rigidity, postural instability or any combination thereof as well as non-motor symptoms including but not limited to mood disorders such as depression, anxiety and irritability, cognitive changes such as with focused attention and planning, slowing of thought, language and memory difficulties, personality changes, dementia, hallucinations and delusions, orthostatic hypotension, sleep disorders such as insomnia, excessive daytime sleepiness (EDS), rapid eye movement behavior disorder (RBD), vivid dreams, talking and moving during sleep, restless legs syndrome (RLS)/periodic leg movements disorder (PLMD), constipation and early satiety, pain, fatigue, vision problems, excessive sweating, increase in dandruff or oily skin, urinary urgency, frequency and incontinence, loss of sense of smell, sexual problems, weight loss or weight gain and impulsive control disorders such as binge eating, excessive shopping or gambling.

In some embodiments, administration of nelotanserin or a pharmaceutically acceptable salt, hydrate, polymorph, or solvate thereof results in an improvement in the individuals Unified Parkinson's Disease Rate Scale (UPDRS) Part II relating to activities of daily living, Part III relating to motor examination or a combination thereof compared to baseline. In some embodiments, administration of nelotanserin or a pharmaceutically acceptable salt, hydrate, polymorph, or solvate thereof results in an improvement in the individuals Unified Parkinson's Disease Rate Scale (UPDRS) Part II relating to activities of daily living, compared to baseline. In some embodiments, parameters observed in UPDRS Part II as activities of daily living, include, but are not limited to speech, salivation, swallowing, handwriting, cutting food and handling utensils, dressing, hygiene, turning in bed or adjusting clothing, falling, freezing when walking, walking, tremor, sensory complaints or any combination thereof. In some embodiments, administration of nelotanserin or a pharmaceutically acceptable salt, hydrate, polymorph, or solvate thereof results in an improvement in the individuals Unified Parkinson's Disease Rate Scale (UPDRS) Part III relating to motor examination or a combination thereof compared to baseline. In some embodiments, parameters observed in UPDRS Part III as related to motor examination include, but are not limited to speech, facial expression, tremor at rest, action tremor, rigidity, finger taps, hand grips, hand pronate/supinate, leg agility, arising from chair, posture, gait, postural stability, bradykinesia and hypokinesia or any combination thereof. In some embodiments, administration of nelotanserin or a pharmaceutically acceptable salt, hydrate, polymorph, or solvate thereof results in a decrease in tremor and rigidity in the subject. In some embodiments, an improvement compared to baseline in any of the parameters disclosed herein may be observed within about 1 to about 4 weeks of treatment. In some embodiments, an improvement compared to baseline in any of the parameters disclosed herein may be observed within about 2 weeks of treatment, an improvement in any of the parameters disclosed above may be observed within about 4 weeks of treatment.

In some embodiments, administration of nelotanserin or a pharmaceutically acceptable salt, hydrate, polymorph, or solvate thereof results in an improvement in the individuals Unified Parkinson's Disease Rate Scale (UPDRS) Part III relating to motor examination compared to baseline. In some embodiments, the UPDRS Part III improvement may be a 1, 2, 3, 4, or 5 point improvement. In some embodiments, a primary efficacy population may have a 3.12 point improvement in the UPDRS Part III. In some embodiments, a prespecified DLB subset may have a 4.0 improvement in the UPDRS Part III.

In some embodiments, treatment, and/or prophylaxis of the Parkinsonian symptoms of dementia with Lewy Bodies, Parkinson's disease, Parkinson's disease dementia, REM sleep behavior disorder, or a combination thereof results in no worsening of the individuals Scale for the Assessment of Positive Symptoms (SAPS) compared to baseline. In some embodiments, treatment, and/or prophylaxis of the Parkinsonian symptoms of dementia with Lewy bodies, Parkinson's disease, Parkinson's disease dementia, REM sleep behavior disorder, or a combination thereof results in an improvement in the individuals Scale for the Assessment of Positive Symptoms (SAPS) compared to baseline. The SAPS is a rating scale to measure positive symptoms in schizophrenia. SAPS is split into 4 domains, and within each domain separate symptoms are rated from 0 (absent) to 5 (severe). In some embodiments, administration of nelotanserin or a pharmaceutically acceptable salt, hydrate, polymorph, or solvate thereof results in no worsening of the individuals score on the Scale for Assessment of Positive Symptoms (SAPS) compared to baseline. In some embodiments, administration of nelotanserin or a pharmaceutically acceptable salt, hydrate, polymorph, or solvate thereof results in an improvement in the individuals score on the Scale for Assessment of Positive Symptoms (SAPS) compared to baseline. In some embodiments, administration of nelotanserin or a pharmaceutically acceptable salt, hydrate, polymorph, or solvate thereof results in no worsening of the individuals score on the Scale for Assessment of Positive Symptoms-Parkinson's disease (SAPS-PD) compared to baseline. In some embodiments, administration of nelotanserin or a pharmaceutically acceptable salt, hydrate, polymorph, or solvate thereof results in an improvement in the individuals score on the Scale for Assessment of Positive Symptoms-Parkinson's disease (SAPS-PD) compared to baseline. In some embodiments, administration of nelotanserin or a pharmaceutically acceptable salt, hydrate, polymorph, or solvate thereof results in no worsening of the in the individuals score on the Scale for Assessment of Positive Symptoms-Parkinson's disease-Hallucinations (SAPS-PD-H) compared to baseline. In some embodiments, administration of nelotanserin or a pharmaceutically acceptable salt, hydrate, polymorph, or solvate thereof results in an improvement in the in the individuals score on the Scale for Assessment of Positive Symptoms-Parkinson's disease-Hallucinations (SAPS-PD-H) compared to baseline. In some embodiments, administration of nelotanserin or a pharmaceutically acceptable salt, hydrate, polymorph, or solvate thereof results in no worsening of the individual's score on the Scale for Assessment of Positive Symptoms Parkinson's disease-Delusions (SAPS-PD-D) compared to baseline. In some embodiments, administration of nelotanserin or a pharmaceutically acceptable salt, hydrate, polymorph, or solvate thereof results in an improvement in the individual's score on the Scale for Assessment of Positive Symptoms Parkinson's disease-Delusions (SAPS-PD-D) compared to baseline. In some embodiments, administration of nelotanserin or a pharmaceutically acceptable salt, hydrate, polymorph, or solvate thereof results in no worsening of the individuals score on the Scale for Assessment of Positive Symptoms Hallucinations/Delusions (SAPS-HD) compared to baseline. In some embodiments, administration of nelotanserin or a pharmaceutically acceptable salt, hydrate, polymorph, or solvate thereof results in an improvement in the individuals score on the Scale for Assessment of Positive Symptoms Hallucinations/Delusions (SAPS-HD) compared to baseline. In some embodiments, administration of nelotanserin or a pharmaceutically acceptable salt, hydrate, polymorph, or solvate thereof results in no worsening of the individuals score on the Scale for Assessment of Positive Symptoms-Hallucinations (SAPS-H) compared to baseline. In some embodiments, administration of nelotanserin or a pharmaceutically acceptable salt, hydrate, polymorph, or solvate thereof results in an improvement in the individuals score on the Scale for Assessment of Positive Symptoms-Hallucinations (SAPS-H) compared to baseline. In some embodiments, administration of nelotanserin or a pharmaceutically acceptable salt, hydrate, polymorph, or solvate thereof results in no worsening of the individuals score on the Scale for Assessment of Positive Symptoms-Delusions (SAPS-D) compared to baseline. In some embodiments, administration of nelotanserin or a pharmaceutically acceptable salt, hydrate, polymorph, or solvate thereof results in an improvement in the individuals score on the Scale for Assessment of Positive Symptoms-Delusions (SAPS-D) compared to baseline. In some embodiments, administration of nelotanserin or a pharmaceutically acceptable salt, hydrate, polymorph, or solvate thereof results in no worsening of the individuals score on the Scale for Assessment of Positive Symptoms-Visual Hallucinations (SAPS-VH) compared to baseline. In some embodiments, administration of nelotanserin or a pharmaceutically acceptable salt, hydrate, polymorph, or solvate thereof results in an improvement in the individuals score on the Scale for Assessment of Positive Symptoms-Visual Hallucinations (SAPS-VH) compared to baseline. In some embodiments, an improvement compared to baseline in any of the parameters disclosed herein may be observed within about 1 to about 4 weeks of treatment. In some embodiments, an improvement compared to baseline in any of the parameters disclosed herein may be observed within about 2 weeks of treatment, an improvement in any of the parameters disclosed above may be observed within about 4 weeks of treatment.

In some embodiments, an individual may have a higher baseline SAP-PD prior to treatment. In further embodiments, an individual may have a higher baseline SAPS-PD and may have a greater nelotanserin treatment effect. In some embodiments, an individual may have a higher baseline SAPS-PD that is greater than 9. In additional embodiments, an individual may have a higher baseline SAPS-PD that is greater than 15. In some embodiments, an individual may have a higher baseline SAPS-PD that is greater than 9 and may have a nelotanserin treatment difference of −2.2 when compared to placebo. In some embodiments, patients may have a higher baseline SAPS-PD that is greater than 15 and may have a nelotanserin treatment difference of −5.5 when compared to placebo.

In some embodiments, treatment, and/or prophylaxis of the Parkinsonian symptoms of dementia with Lewy Bodies, Parkinson's diseases, Parkinson's disease dementia, REM sleep behavior disorder, or a combination thereof results in an improvement in the individual's Mini-Mental State Examination score, cognition, attention, Clinician's Interview-Based Impression of Change with caregiver input (CIBIC+) rating, neuropsychiatric inventory (NPI), North-East Visual Hallucinations Interview (NEVHI), Cognitive Drug Research (CDR) computerized assessment system, Scale for the Assessment of Positive Symptoms (SAPS), Positive and Negative Syndrome Scale (PANSS), Clinical Global Impression (CGI) scale or any combination thereof. In some embodiments, treating or prophylaxis results in fluctuations in cognition, attention or a combination thereof.

Antipsychotics are typically used to treat the neuropsychiatric symptoms of psychosis such as delusions, hallucinations, paranoia, or confused thoughts. They are also used in the treatment of schizophrenia, severe depression and severe anxiety and for stabilizing episodes of mania in people with Bipolar Disorder. Treatment with antipsychotics can however result in the manifestation of parkinsonian symptoms in patients being treated for the conditions described above. Accordingly, these patients may benefit from an agent that can treat, and, or prevent these parkinsonian symptoms. The present invention is also directed to the use of a 5-HT_{2A} serotonin receptor inverse agonist for the prophylaxis and/or treatment of the parkinsonian symptoms caused by the use of antipsychotic agents in an individual being treated for a neuropsychiatric symptoms of a condition, comprising administering to said individual in need thereof a therapeutically effective amount of said 5-HT_{2A} serotonin receptor inverse agonist. In some embodiments, the conditions may include psychoses such as delusions, hallucinations, paranoia, or confused thoughts, schizophrenia, severe depression, severe anxiety, for stabilizing episodes of mania in people with bipolar disorder or any combination thereof. In some embodiments, administering to said individual in need thereof a therapeutically effective amount of said 5-HT_{2A} serotonin receptor inverse agonist does not result in a worsening of neuropsychiatric symptoms of the condition such as, but not limited to hallucinations, delusions, or a combination thereof. In some embodiments, administering to said individual in need thereof a therapeutically effective amount of said 5-HT_{2A} serotonin receptor inverse agonist results in an improvement of neuropsychiatric symptoms such as, but not limited to hallucinations, delusions, or combination thereof. In some embodiments, administering to said individual in need thereof a therapeutically effective amount of said 5-HT_{2A} serotonin receptor inverse agonist results in an improvement of neuropsychiatric symptoms and Parkinsonian symptoms. In some embodiments, administering to said individual in need thereof a therapeutically effective amount of said 5-HT_{2A} serotonin receptor inverse agonist does not result in QT interval prolongation. In some embodiments, administering to said individual in need thereof a therapeutically effective amount of said 5-HT_{2A} serotonin receptor inverse agonist results in an improvement of Parkinsonian symptoms. In some embodiments, administering to said individual in need thereof a therapeutically effective amount of said 5-HT_{2A} serotonin receptor inverse agonist results in an improvement of Parkinsonian motor symptoms.

In some embodiments, the methods described herein can be used in the prophylaxis and/or treatment of the parkinsonian symptoms caused by the use of both typical (defined as an agent that is a selective dopamine D₂ receptor blocker) or atypical antipsychotic agents (defined as agents with mixed receptor selectivity). Typical antipsychotic agents include but are not limited to chlorpromazine, chlorprothixene, levomepromazine, mesoridazine, periciazine, promazine, thioridazine, loxapine, molindone, perphenazine, thiothixene, droperidol, flupentixol, fluphenazine, haloperidol, pimozide, prochlorperazine, thioproperazine, trifluoperazine, and zuclopenthixol. Atypical antipsychotics include but are not limited to aripiprazole, asenapine, brexpiprazole, cariprazine, clozapine, iloperidone, lurasidone, olanzapine, paliperidone, risperidone, ziprasidone, quetiapine, clozapine, risperidone, and clonazepam.

One aspect of the present invention pertains to the use of pharmaceutical compositions comprising: (a) nelotanserin or pharmaceutically acceptable salts, hydrates, polymorphs or solvates thereof; and (b) an excipient. One aspect of the present invention pertains to the use of pharmaceutical compositions comprising: (a) nelotanserin or pharmaceutically acceptable salts, hydrates, polymorphs or solvates thereof; and (b) an excipient selected from PVP and coPVP.

One aspect of the present invention pertains to kits for the prophylaxis and/or treatment of the symptoms of dementia with Lewy bodies, Parkinson's disease, Parkinson's disease dementia, REM sleep behavior disorder, or a combination thereof in an individual comprising a container and a pharmaceutical composition of the present invention.

One aspect of the present invention pertains to methods for the prophylaxis and/or treatment of the symptoms of dementia with Lewy bodies, Parkinson's disease, Parkinson's disease dementia, REM sleep behavior disorder, or a combination thereof in an individual comprising administering to the individual in need thereof a therapeutically effective amount of a pharmaceutical composition of the present invention.

In some embodiments, the pharmaceutical composition is administered orally, nasally sublingually, buccally, transdermally, vaginally or rectally. In some embodiments, the pharmaceutical composition is administered orally.

One aspect of the present invention pertains to the use of a pharmaceutical composition of the present invention in the manufacture of a medicament for the prophylaxis and/or treatment of the symptoms of dementia with Lewy bodies, Parkinson's disease, or a combination thereof.

In some embodiments, the individual is concurrently receiving a therapeutically effective amount of at least one additional therapeutic agent selected from the group consisting of aripiprazole, asenapine, brexpiprazole, cariprazine, clozapine, iloperidone, lurasidone, olanzapine, paliperidone, risperidone, ziprasidone, pimavanserin, chlorpromazine, chlorprothixene, levomepromazine, mesoridazine, periciazine, promazine, thioridazine, loxapine, molindone, perphenazine, thiothixene, droperidol, flupentixol, fluphenazine, haloperidol, pimozide, prochlorperazine, thioproperazine, trifluoperazine, zuclopenthixol, melatonin, quetiapine, clozapine, risperidone, clonazepam, levodopa, carbidopa, an antiparkinsonian drug, an acetylcholinesterase inhibitor, NMDA receptor antagonist, an atypical antipsychotic agent, a dopaminergic agent, a benzodiazepine, an antidepressant, and a combination thereof. In some embodiments, the therapeutically effective amount of melatonin is about 1 mg to about 5 mg. In some embodiments, the therapeutically effective amount of quetiapine is about 12.5 mg to about 100 mg. In some embodiments, the therapeutically effective amount of quetiapine is less than about 25 mg. In some embodiments, the therapeutically effective amount of clonazepam is about 0.0625 mg to about 5 mg. In some embodiments, the antiparkinsonian drug is selected from an MAO-B inhibitor, a COMT inhibitor, a dopamine agonist or any combination thereof. In some embodiments, the therapeutically effective amount of levodopa or a pharmaceutically acceptable salt, hydrate, polymorph, or solvate thereof is from about 0.001 mg to about 10,000 mg, or about 0.001 mg to about 8,000 mg. In some embodiments, the therapeutically effective amount of levodopa or a pharmaceutically acceptable salt, hydrate, polymorph, or solvate thereof is about 285 mg, about 300 mg, about 400 mg, about 435 mg, about 500 mg, about 585 mg, about 600 mg, about 700 mg, about 735 mg, about 750 mg, about 800 mg, about 980 mg, about 1,000 mg, about 1,225 mg, about 1,250 mg, about 1,470 mg, about 1,500 mg, about 1,715 mg, about 1,750 mg, about 1,960 mg, about 2,000 mg, about 2,205 mg, about 2,250 mg, about 2,450 mg, about 2,500 mg, about 2,750 mg, about 3,000 mg, about 3,250 mg, about 3,500 mg, about 3,750 mg, about 4,000 mg, about 4,250 mg, about 5,000 mg, about 5,250 mg, about 5,500 mg, about 5,750 mg, about 6,000 mg, about 6,250 mg, about 6,500 mg, about 6,750 mg, about 7,000 mg, about 7,250 mg, about 7,500 mg, about 7,750 mg, or about 8,000 mg. In some embodiments, the therapeutically effective amount of carbidopa or a pharmaceutically acceptable salt, hydrate, polymorph, or solvate thereof is configured for immediate release, for extended release, for delayed release, or any combination thereof. In some embodiments, the therapeutically effective amount of carbidopa is from about 0.001 mg to about 1,000 mg, or from about 0.001 mg to about 700 mg. In some embodiments, the therapeutically effective amount of carbidopa is about 30 mg, about 40 mg, about 50 mg, about 60 mg, about 70 mg, about 71.25 mg, about 80 mg, about 108.75 mg, about 146.25 mg, 183.75 mg, about 245 mg, about 245 mg, about 306.25 mg, about 367.5 mg, about 428.75 mg, about 490 mg, about 551.25 mg, or about 612.5 mg. In some embodiments, the acetylcholinesterase inhibitor is selected from the group consisting of donepezil, rivastigmine, galantamine, and pharmaceutically acceptable salts, hydrates, polymorphs, or solvates thereof. In some embodiments, the acetylcholinesterase inhibitor is donepezil or a pharmaceutically acceptable salt, hydrate, polymorph, or solvate thereof. In some embodiments, the therapeutically effective amount of donepezil or a pharmaceutically acceptable salt, hydrate, polymorph, or solvate thereof is configured for immediate release, for extended release, for delayed release, or any combination thereof. In some embodiments, the therapeutically effective amount of donepezil or a pharmaceutically acceptable salt, hydrate, polymorph, or solvate thereof is from about 0.001 mg to about 1,000 mg, or about 0.001 mg to about 30 mg. In some embodiments, the therapeutically effective amount of donepezil or a pharmaceutically acceptable salt, hydrate, polymorph, or solvate thereof is about 5 mg, 10 mg, or 23 mg. In some embodiments, the acetylcholinesterase inhibitor is rivastigmine or a pharmaceutically acceptable salt, hydrate, polymorph, or solvate thereof. In some embodiments, the therapeutically effective amount of rivastigmine or a pharmaceutically acceptable salt, hydrate, polymorph, or solvate thereof is from about 0.001 mg to about 1,000 mg, or about 0.001 mg to about 15 mg. In some embodiments, the therapeutically effective amount of rivastigmine or a pharmaceutically acceptable salt, hydrate, polymorph, or solvate thereof is about 1.5 mg, about 3 mg, about 4.5 mg, about 6 mg, about 9 mg, about 9.5 mg, about 12 mg, or about 13.3 mg. In some embodiments, the therapeutically effective amount of rivastigmine or a pharmaceutically acceptable salt, hydrate, polymorph, or solvate thereof is configured for immediate release, for extended release, for delayed release, or any combination thereof. In some embodiments, the acetylcholinesterase inhibitor is galantamine or a pharmaceutically acceptable salt, hydrate, polymorph, or solvate thereof. In some embodiments, the therapeutically effective amount of galantamine or a pharmaceutically acceptable salt, hydrate, polymorph, or solvate thereof is configured for immediate release, for extended release, for delayed release, or any combination thereof. In some embodiments, the therapeutically effective amount of galantamine or a pharmaceutically acceptable salt, hydrate, polymorph, or solvate thereof is from about 0.001 mg to about 1,000 mg, or about 0.001 mg to about 30 mg. In some embodiments, the therapeutically effective amount of galantamine or a pharmaceutically acceptable salt, hydrate, polymorph, or solvate thereof is about 4 mg, about 8 mg, about 12 mg, about 16 mg, or about 24 mg. In some embodiments, NMDA receptor antagonist is selected from the group consisting of memantine, amantadine, ketamine, and pharmaceutically acceptable salts, hydrates, polymorphs, or solvates thereof. In some embodiments, the NMDA receptor antagonist is memantine or a pharmaceutically acceptable salt, hydrate, polymorph, or solvate thereof. In some embodiments, the therapeutically effective amount of memantine or a pharmaceutically acceptable salt, hydrate, polymorph, or solvate thereof is configured for immediate release, for extended release, for delayed release, or any combination thereof. In some embodiments, the therapeutically effective amount of memantine or a pharmaceutically acceptable salt, hydrate, polymorph, or solvate thereof is from about 0.001 mg to about 1,000 mg, or about 0.001 mg to about 30 mg. In some embodiments, the therapeutically effective amount of memantine or a pharmaceutically acceptable salt, hydrate, polymorph, or solvate thereof is about 5 mg, about 7 mg, about 10 mg, about 14 mg, about 20 mg, about 21 mg, or about 28 mg. In some embodiments, the therapeutically effective amount of memantine or a pharmaceutically acceptable salt, hydrate, polymorph, or solvate thereof is configured for extended release, for delayed release or a combination thereof. In some embodiments, the NMDA receptor antagonist is amantadine or a pharmaceutically acceptable salt, hydrate, polymorph, or solvate thereof. In some embodiments, the therapeutically effective amount of amantadine or a pharmaceutically acceptable salt, hydrate, polymorph, or solvate thereof is configured for immediate release, for extended release, for delayed release, or any combination thereof. In some embodiments, the therapeutically effective amount of amantadine or a pharmaceutically acceptable salt, hydrate, polymorph, or solvate thereof is from about 0.001 mg to about 1,000 mg, or about 0.001 mg to about 500 mg. In some embodiments, the therapeutically effective amount of amantadine or a pharmaceutically acceptable salt, hydrate, polymorph, or solvate thereof is from about 100 mg to about 400 mg. In some embodiments, the therapeutically effective amount of amantadine or a pharmaceutically acceptable salt, hydrate, polymorph, or solvate thereof is about 100 mg, about 200 mg, about 300 mg or about 400 mg.

In some embodiments, the at least one additional therapeutic agent is a 5HT₆ antagonist. In some embodiments, the at least one additional therapeutic agent is 3-phenylsulfonyl-8-piperazinyl-1yl-quinoline. In some embodiments, 3-phenylsulfonyl-8-piperazinyl-1yl-quinoline is administered in a therapeutically effective amount. In some embodiments, the therapeutically effective amount of 3-phenylsulfonyl-8-piperazinyl-1yl-quinoline or pharmaceutically acceptable salts, hydrates, polymorphs or solvates thereof is configured for extended release, and the additional therapeutic agent useful for treating a neurodegenerative disease is configured for immediate release, for sustained release, for extended release, or any combination thereof. In some embodiments, the therapeutically effective amount of 3-phenylsulfonyl-8-piperazinyl-1yl-quinoline or a pharmaceutically acceptable salt, hydrate, polymorph, or solvate thereof is from about 0.001 mg to about 1,000 mg, about 0.001 mg to about 200 mg, about 0.001 mg to about 175 mg, or 0.001 mg to about 70 mg. In some embodiments, the therapeutically effective amount of 3-phenylsulfonyl-8-piperazinyl-1yl-quinoline or a pharmaceutically acceptable salt, hydrate, polymorph, or solvate thereof is about 15 mg, about 35 mg, or about 70 mg.

In some embodiments, the at least one additional therapeutic agent is a monoclonal antibody. In some embodiments, the second therapeutic agent is a human monoclonal antibody. In some embodiments, the second therapeutic agent is a humanized monoclonal antibody. In some embodiments the monoclonal antibody targets beta amyloid. In some embodiments the beta amyloid may comprise aggregated beta amyloid such as but not limited to soluble oligomers, insoluble fibrils deposited into amyloid plaque, or a combination thereof. In some embodiments, the monoclonal antibody is Aducanumab (BIIB037), Gantenerumab, Bapineuzumab, Crenezumab, Ponezumab, Solanezumab, SAR228810, MEDI1814, BAN2401, or any combination thereof. In some embodiments, the monoclonal antibody targets alpha-synuclein. In some embodiments, the monoclonal antibody targeting alpha-synuclein is RG-7935, Posiphen, Affitope PD03A, Affitope PD01A, or any combination thereof.

In some embodiments, the at least one additional therapeutic agent is a BACE enzyme inhibitor. In some embodiments, the BACE enzyme inhibitor is CTS-21166, MK-8931, AZD3293, LY3314814, BI1181181, LY2886721, E2609, RG7129, JNJ-5486911, TAK-070, or any combination thereof.

In some embodiments, the at least one additional therapeutic agent is a RAGE inhibitor. In some embodiments, the RAGE inhibitor is TTP488 (Azeliragon), TTP4000, FPS-ZM1, or any combination thereof.

In some embodiments, the at least one additional therapeutic agent is an antibody targeting Tau. In some embodiments, the antibody targeting Tau is AADVAC-1, AADVAC-2, ACI-35, BMS-986168, RG7345, TRx-237-015 (LMTX), AV-1451, AV-680, Posiphen, or any combination thereof.

In some embodiments, the at least one additional therapeutic agent is a α7 nicotinic acetylcholine receptor modulator. In some embodiments, the α7 nicotinic acetylcholine receptor modulator is Encenicline (EVP-6124), ABT-126, ABT 418, RG3487, Varenicline, A-867744, TC-5219, AVL3288, BMS933043, DSP-3748, or any combination thereof.

In some embodiments, the at least one additional therapeutic agent may include one or more treatments for Alzheimer's disease such as Namzaric™, Exelon®, Aricept® (donepezil hydrochloride), Namenda® (memantine hydrochloride), or galantamine hydrobromide. In some embodiments, described compositions and formulations may be administered in combination with one or more treatments for Parkinson's Disease such as ABT-126 (Abbott Laboratories), pozanicline (Abbott Laboratories), MABT-5102A (AC Immune), Affitope AD-01 (AFFiRiS GmbH), Affitope AD-02 (AFFiRiS GmbH), davunetide (Allon Therapeutics Inc.), nilvadipine derivative (Archer Pharmaceuticals), Anapsos (ASAC Pharmaceutical International AIE), ASP-2535 (Astellas Pharma Inc.), ASP-2905 (Astellas Pharma Inc.), 1 1C-AZD-2184 (AstraZeneca pic), 1 1C-AZD-2995 (AstraZeneca pic), 18F-AZD-4694 (AstraZeneca pic), AV-965 (Avera Pharmaceuticals Inc.), AVN-101 (Avineuro Pharmaceuticals Inc.), immune globulin intravenous (Baxter International Inc.), EVP-6124 (Bayer AG), nimodipine (Bayer AG), BMS-708163 (Bristol-Myers Squibb Co), CERE-110 (Ceregene Inc.), CLL-502 (CLL Pharma), CAD-106 (Cytos Biotechnology AG), mimopezil ((Debiopharm SA), DCB-AD1 (Development Centre for Biotechnology), EGb-761 (Dr Willmar Schwabe GmbH & Co), E-2012 (Eisai Co Ltd), ACC-001 (Elan Corp pic), bapineuzumab (Elan Corp pic), ELND-006 (Elan Pharmaceuticals Inc.), atomoxetine (Eli Lilly & Co), LY-2811376 (Eli Lilly & Co), LY-451395 (Eli Lilly & Co), m266 (Eli Lilly & Co), semagacestat (Eli Lilly & Co), solanezumab (Eli Lilly & Co), AZD-103 (Ellipsis Neurotherapeutics Inc.), FGLL (ENKAM Pharmaceuticals A/S), EHT-0202 (ExonHit Therapeutics SA), celecoxib (GD Searle & Co), GSK-933776A (GlaxoSmithKline Plc, rosiglitazone XR (GlaxoSmithKline pic), SB-742457 (GlaxoSmithKline pic), R-1578 (Hoffmann-La Roche AG), HF-0220 (Hunter-Fleming Ltd), oxiracetam (ISF Societa Per Azioni), KD-501 (Kwang Dong Pharmaceutical Co Ltd), NGX-267 (Life Science Research Israel), huperzine A (Mayo Foundation), Dimebon (Medivation Inc.), MEM-1414 (Memory Pharmaceuticals Corp), MEM-3454 (Memory Pharmaceuticals Corp), MEM-63908 (Memory Pharmaceuticals Corp), MK-0249 (Merck & Co Inc.), MK-0752 (Merck & Co Inc.), simvastatin (Merck & Co Inc.), V-950 (Merck & Co Inc.), memantine (Merz & Co GmbH), neramexane (Merz & Co GmbH), Epadel (Mochida Pharmaceutical Co Ltd), 123I-MNI-330 (Molecular Neuroimaging Lie), gantenerumab (MorphoSys AG), NIC5-15 (Mount Sinai School of Medicine), huperzine A (Neuro-Hitech Inc.), OXIGON (New York University), NP-12 (Noscira SA), NP-61 (Noscira SA), rivastigmine (Novartis AG), ECT-AD (NsGene A/S), arundic acid (Ono Pharmaceutical Co Ltd), PF-3084014 (Pfizer Inc.), PF-3654746 (Pfizer Inc.), RQ-00000009 (Pfizer Inc.), PYM-50028 (Phytopharm pic), Gero-46 (PN Gerolymatos SA), PBT-2 (Prana Biotechnology Ltd), PRX-03140 (Predix Pharmaceuticals Inc.), Exebryl-1 (ProteoTech Inc.), PF-4360365 (Rinat Neuroscience Corp), HuCAL anti-beta amyloid monoclonal antibodies (Roche AG), EVT-302 (Roche Holding AG), nilvadipine (Roskamp Institute), galantamine (Sanochemia Pharmazeutika AG), SAR-110894 (sanofi-aventis), INM-176 (Scigenic & Scigen Harvest), mimopezil (Shanghai Institute of Materia Medica of the Chinese Academy of Sciences), NEBO-178 (Stegram Pharmaceuticals), SUVN-502 (Suven Life Sciences), TAK-065 (Takeda Pharmaceutical), ispronicline (Targacept Inc.), rasagiline (Teva Pharmaceutical Industries), T-817MA (Toyama Chemical), PF-4494700 (TransTech Pharma Inc.), CX-717 (University of California), 18F-FDDNP (University of California Los Angeles), GTS-21 (University of Florida), 18F-AV-133 (University of Michigan), 18F-AV-45 (University of Michigan), tetrathiomolybdate (University of Michigan), 1231-IMPY (University of Pennsylvania), 18F-AV-1/ZK (University of Pennsylvania), 11C-6-Me-BTA-1 (University of Pittsburgh), 18F-6-0H-BTA-1 (University of Pittsburgh), MCD-386 (University of Toledo), leuprolide acetate implant (Voyager Pharmaceutical Corp), aleplasinin (Wyeth), begacestat (Wyeth), GSI-136 (Wyeth), NSA-789 (Wyeth), SAM-531 (Wyeth), CTS-21166 (Zapaq), and ZSET-1446 (Zenyaku Kogyo).

In some embodiments, the at least one additional therapeutic agent may include one or more agents useful for the treatment of motor neuronal disorders, such as AEOL-10150 (Aeolus Pharmaceuticals Inc.), riluzole (Aventis Pharma AG), ALS-08 (Avicena Group Inc.), creatine (Avicena Group Inc.), arimoclomol (Biorex Research and Development Co), mecobalamin (Eisai Co Ltd), talampanel (Eli Lilly & Co), R-7010 (F Hoffmann-La Roche Ltd), edaravone (Mitsubishi-Tokyo Pharmaceuticals Inc.), arundic acid (Ono Pharmaceutical Co Ltd), PYM-50018 (Phytopharm pic), RPI-MN (ReceptoPharm Inc.), SB-509 (Sangamo Biosciences Inc.), olesoxime (Trophos SA), sodium phenylbutyrate (Ucyclyd Pharma Inc.), and R-pramipexole (University of Virginia).

In some embodiments, the at least one additional therapeutic agent may be an agent known to modify cholinergic transmission such as M1 muscarinic receptor agonists or allosteric modulators, M2 muscarinic antagonists, acetylcholinesterase inhibitors, nicotinic receptor agonists or allosteric modulators, 5-HT₄ receptor partial agonists or 5HT_{1A} receptor antagonists and NMDA receptor antagonists or modulators, glutamate antagonists, GABA-ergic antagonists, H3 antagonists, putative metabolic/mitochondrial modulators, or disease modifying agents such as β or γ-secretase inhibitors, Tau-targeted therapeutics, β-amyloid aggregation inhibitors and β-amyloid immunotherapies, an antidepressants, for example a tricyclic, a MAOI (Monoamine oxidase inhibitor) a SSRI (Selective Serotonin Reuptake Inhibitor), a SNRI (Serotonin and Noradrenaline Reuptake Inhibitor) or a NaSSA (noradrenergeric and specific serotonergic antidepressant). Examples of specific antidepressant compounds include amitriptyline, clomipramine, citalopram, dosulepin, doxepin, fluoxetine, imipramine, lofepramine, mirtazapine, moclobemide, nortriptyline, paroxetine, phenelzine, reboxetine, sertraline, tranylcypromine, trazodone, or venlafaxine. In some embodiments, additional therapeutic agents may include antipsychotic drugs, such as olanzapine, clozapine, risperidone, quetiapine, aripiprazole or paliperiden.

In some embodiments, the at least one additional therapeutic agent may be combination of an agent known to be a sigma-1 receptor agonist and an uncompetitive NMDA receptor antagonist. In some embodiments, a sigma-1 receptor agonist and an uncompetitive NMDA receptor antagonist may be Dextromethorphan hydrobromide and quinidine sulfate which is marketed as Nuedexta®. In some embodiments, the daily dose of dextromethorphan hydrobromide is about 20 mg and the dose of quinidine sulfate is about 10 mg. In some embodiments, the daily dose of dextromethorphan hydrobromide is about 40 mg and the dose of quinidine sulfate is about 20 mg.

In some embodiments, the at least one additional agent may be a combination of an acetylcholinesterase inhibitor and an anti-cholinergic agent. In some embodiments, the acetylcholinesterase inhibitor is selected from the group consisting of donepezil, rivastigmine, galantamine, tacrine, physostigmine, pyridostigmine, neostigmine, icopezil, zanapezil, ipidacrine, phenserine, ambenonium, edrophonium, ladostigil, huperzine A, pyridostigmine, ambenonium, demecarium, a phenanthrene derivative, caffeine, a piperidine tacrine (also known as tetrahydroaminoacridine), edrophonium, ladostigil, ungeremine, lactucopicrin, 6-[(3-cyclobutyl-2,3,4,5-tetrahydro-1H-3-benzazepin-7-yl)oxy]-N-methyl-3-pyridinecarboxamide hydrochloride or 1-{6-[(3-cyclobutyl-2,3,4,5-tetrahydro-1H-3-benzazepin-7-yl)oxy]-3-pyridinyl}-2-pyrrolidinone or pharmaceutically acceptable salts, hydrates, polymorphs, or solvates thereof.

In some embodiments, the anti-cholinergic agent is selected from the group consisting of quaternary ammonium anti-cholinergic muscarinic receptor antagonist, a quaternary ammonium non-selective peripheral Anti-Cholinergic agent, a sulfonium non-selective peripheral Anti-Cholinergic agent, a non-selective peripheral muscarinic anti-cholinergic agent, (1 S)-(3R)-1-azabicyclo[2.2.2]oct-3-yl 3,4-dihydro-1-phenyl-2(1H)-iso-quinolinecarboxylate (solifenacin) or pharmaceutically acceptable salts, hydrates, polymorphs, or solvates thereof, 1-methylpiperidin-4-yl) 2,2-di(phenyl)-2-propoxyacetate (propiverine) or pharmaceutically acceptable salts, hydrates, polymorphs, or solvates thereof, 1,4,5,6-tetrahydro-1-methylpyrimidin-2-ylmethyl α-cyclohexyl-α-hydroxy-α-phenyl acetate (oxyphencyclimine) or pharmaceutically acceptable salts, hydrates, polymorphs, or solvates thereof, (R)—N,N-diisopropyl-3-(2-hydroxy-5-methylphenyl)-3-phenylpropanamide (tolterodine) or pharmaceutically acceptable salts, hydrates, polymorphs, or solvates thereof. In some embodiments, the quaternary ammonium anti-cholinergic muscarinic receptor antagonist is selected from trospium and glycopyrrolate or pharmaceutically acceptable salts, hydrates, polymorphs, or solvates thereof. In some embodiments, the non-selective peripheral muscarinic anti-cholinergic agent is solifenacin or pharmaceutically acceptable salts, hydrates, polymorphs, or solvates thereof. In some embodiments, the anti-cholinergic agent is a compound of formula I:

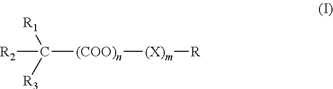

or pharmaceutically acceptable salts, hydrates, polymorphs, or solvates thereof, wherein R is a radical selected from the group consisting of those of formulas (a)-(e)

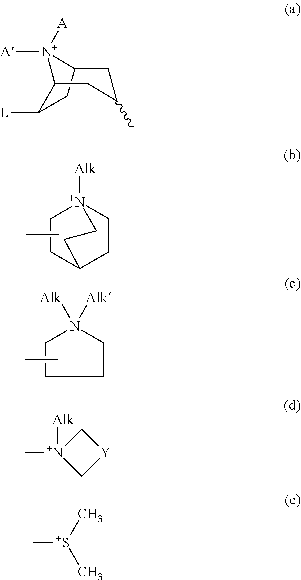

A being methyl and A′ being (C1-C4)alkyl or 2-fluoroethyl group or A and A′ forming a 1,4-butylene or 1,5-pentylene chain, L being hydrogen or methoxy, Alk and Alk′ each being (C1-C4)alkyl and Y being a bivalent radical selected from the group consisting of 1,2-ethylene, 1,3-propylene, 1,4-butylene and 2-oxa-1,3-propylene; the corresponding counter ion being a pharmaceutically acceptable anion, such as a chloro, bromo, iodo, tartrate, hydrogen tartrate, succinate, maleate, fumarate, sulfate, hydrogen sulfate or methylsulfate anion;

n and m, independently, are zero or 1;

X is a (C2-C3)alkylene group;

R1 and R2 are each phenyl, cyclopentyl, cyclohexyl, 1-cyclohexenyl, 2-thienyl and, when R is a radical (a), also each represents (C1-C4)alkyl;

R3 is H or OH or, only when R is a radical (a), also a COOAlk group, Alk being a (C1-C4)alkyl group.

In some embodiments, R=(a), A=A′=CH3, L=H; n=1; m=0; R1=R2=n-C3H7; and R3=H. In some embodiments, R=(a), A=CH3, A′=isopropyl, L=H; n=1; m=0; R1=phenyl; R2=cyclopentyl; and R3=H. In some embodiments, R=(a), A=CH₃, A′=2-fluoroethyl, L=H; n=1; m=0; R₁═R₂=phenyl; and R₃═OH. In some embodiments, R=(a), A=A′=CH₃, L=H; n=1; m=0; R=phenyl; and R₂═R₃═H. In some embodiments, R=(a), A=CH₃, A′=isopropyl, L=H; n=1; m=0; R₁═R₂=n-C₃H₇; and R₃═H. In some embodiments, R=(a), A=A′=CH₃, L=H; n=1; m=0; R₁=phenyl; R₂═COOC₂H₅; and R₃═H. In some embodiments, R=(a), A=A′=CH₃, L=methoxy; n=1; m=0; R₁═R₂=phenyl, and R₃═OH. In some embodiments, R=(a), A+A′=1,4-butylene, L=H; n=1; m=0; R₁═R₂=phenyl; and R₃=0H. In some embodiments, R=(b)-3-, Alk=methyl; n=1; m=0; R₁═R₂=phenyl; and R₃═OH. In some embodiments, R=(b)-3-, Alk=methyl; n=1; m=0; R₁=phenyl; R₂=cyclopentyl; and R₃═OH. In some embodiments, R=(c)-3-, both Alk and Alk′=ethyl; n=1; m=0; R₁═R₂=phenyl; and R₃═OH. In some embodiments, R=(c)-3-, both Alk and Alk′=methyl; n=1; m=0; R₁═R₂=phenyl; and R₃═OH. In some embodiments, R=(c)-3-, Alk=methyl and Alk′=ethyl; n=1; m=0; R₁=phenyl; R₂=cyclopentyl; and R₃═H. In some embodiments, R=(c)-3-, both Alk and Alk′=methyl; n=1; m=0; R₁=phenyl; R₂=cyclopentyl; and R₃═H. In some embodiments, R=(c)-3-, both Alk and Alk′=methyl n=1; m=0; R₁=phenyl; R₂=2-thienyl; and R₃═OH. In some embodiments, R=(c)-3-, both Alk and Alk′=methyl; n=1; m=0; R₁=phenyl; R₂=cyclohexyl; and R₃═H. In some embodiments, R=(c)-2-, both Alk and Alk′=methyl; n=1; m=1; X=1,2-ethylene; R₁=phenyl; R₂=cyclohexyl; and R₃═OH. In some embodiments, R=(c)-3-, both Alk and Alk′=methyl; n=1; m=0; R₁=phenyl; R₂=cyclopentyl; and R₃═OH. In some embodiments, R=(c)-3-, both Alk and Alk′=methyl; n=1; m=0; R₁=phenyl; R₂=cyclopentyl; R₃═OH. In some embodiments, R=(d), Alk=methyl, Y=1,2-ethylene; n=1; m=1; X=1,2-ethylene; R₁═R₂=phenyl; and R₃=0H. In some embodiments, R=(d), Alk=CH₃, Y=1,3-propylene; n=0; m=1; X=1,2-ethylene; R₁=phenyl; R₂=1-cyclohexenyl; and R₃═H. In some embodiments, R=(d), Alk=methyl, Y=1,2-ethylene; n=0; m=1; X=1,2-ethylene; R₁=phenyl; R₂=cyclohexyl; and R₃═OH. In some embodiments, R=(d), Alk=methyl, Y=2-oxa-1,3-propylene; n=0; m=1; X=1,2-ethylene; R₁=phenyl; R₂=2-thienyl; and R₃═OH. In some embodiments, R=(e); n=1; m=1; X=1,2-ethylene; R₁=phenyl; R₂=cyclohexyl; and R₃═H. In some embodiments, R=(e); n=1; m=1; X=1,2-ethylene; R₁=phenyl; R₂=cyclohexyl; and R₃═OH.

In some embodiments, the anti-cholinergic agent is selected from the group consisting of anisotropine methylbromide, ciclotropium bromide, flutropium bromide, homatropine methylbromide, sintropium bromide, tematropium metilsulfate, tropenziline bromide, trospium chloride, clidinium bromide, droclidinium bromide, benzilonium bromide, benzopyrronium bromide, cyclopyrronium bromide, glycopyrronium bromide (glycopyrrolate), heteronium bromide, hexopyrronium bromide, oxypyrronium bromide, ritropirronium bromide, etipirium iodide, fenclexonium methylsulfate, tricyclamol chloride (procyclidine methochloride), tiemonium iodide, hexasonium iodide, and oxysonium iodide or pharmaceutically acceptable salts, hydrates, polymorphs, or solvates thereof.

In some embodiments, the anti-cholinergic agent is selected from the group consisting of Azoniaspiro[3β-benziloyloxy-(1α,5α-nortropane-8,1′-pyrrolidine]chloride (formula II, A+A′=1,4-butylene) described in U.S. Pat. No. 3,480,626, known under its International Non-proprietary Name trospium chloride, the tartrate, maleate, fumarate and succinate salts of trospium, solifenacin, described in U.S. Pat. No. 6,017,927 and the compound thereof with succinic acid, propiverine, described in DD 106643, and the hydrochloride thereof, oxyphencyclimine, described in GB 795758, and the hydrochloride thereof, tolterodine, described in U.S. Pat. No. 5,382,600, and the hydrogen tartrate thereof.

In some embodiments, the anti-cholinergic agent is selected from the group consisting of a pharmaceutically acceptable salt of trospium, especially trospium chloride, succinate, maleate, fumarate or tartrate, a pharmaceutically acceptable salt of solifenacin, especially its compound with succinic acid 1:1, a pharmaceutically acceptable salt of propiverine, especially its hydrochloride, a pharmaceutically acceptable salt of oxyphencyclimine, especially its hydrochloride or a pharmaceutically acceptable salt of tolterodine, especially its L-hydrogen tartrate. In some embodiments, suitable anti-cholinergic agents include, but are not limited to clinidium, cimetidine, ranitidine, digoxin, scopolamine, dantrolene, chlordiazepoxide, atropine, nifedipine, amantadine, propantheline, propantheline, furosemide, amoxapine, paroxetine, disopyramide, hydroxyzine, diphenhydramine, orphenadrine, olanzapine, clozapine, chlorpheniramine, desipramine, doxepin, biperiden, oxybutynin, benzatropine, promethazine, imipramine, nortriptyline, protriptyline, prochlorperazine, cyclobenzaprine, trihexyphenidyl, cyproheptadine, clomipramine, amitriptyline, chlorpromazine, tolterodine, meclizine, dicyclomine, and thioridazine or pharmaceutically acceptable salts, hydrates, polymorphs, or solvates thereof.

In some embodiments, the combination of an acetylcholinesterase inhibitor and an anti-cholinergic agent is donepezil and glycopyrrolate. In some embodiments, the combination of an acetylcholinesterase inhibitor and an anti-cholinergic agent is donepezil and trospium. In some embodiments, the combination of an acetylcholinesterase inhibitor and an anti-cholinergic agent is rivastigmine and glycopyrrolate. In some embodiments, the combination of an acetylcholinesterase inhibitor and an anti-cholinergic agent is rivastigmine and trospium. In some embodiments, the combination of an acetylcholinesterase inhibitor and an anti-cholinergic agent is donepezil and solifenacin.

In some embodiments, the 5-HT_{2A} serotonin receptor inverse agonist is nelotanserin. In some embodiments, the 5-HT_{2A} serotonin receptor inverse agonist is nelotanserin and nelotanserin may be more potent than another 5-HT_{2A} serotonin receptor inverse agonist in head-to-head 5-HT-induced functional assays. In some embodiments, the 5-HT_{2A} serotonin receptor inverse agonist is nelotanserin and nelotanserin may be more potent than the 5-HT_{2A} serotonin receptor inverse agonist pimavanserin in head-to-head 5-HT-induced functional assays. In some embodiments, the 5-HT_{2A} serotonin receptor inverse agonist is nelotanserin and nelotanserin may be more potent than the 5-HT_{2A} serotonin receptor inverse agonist pimavanserin in intracellular calcium release assays. In further embodiments, nelotanserin may be about 2-fold more potent than pimavanserin.

In some embodiments, the 5-HT_{2A} serotonin receptor inverse agonist is nelotanserin. In some embodiments, pharmacokinetic parameters may support a daily dosing of nelotanserin. In some embodiments, the T_{1/2} of nelotanserin may be about 15 hours.

In some embodiments, the HT_{2A} serotonin receptor inverse agonist is nelotanserin. In some embodiments, nelotanserin may not inhibit cytochrome enzymes. In further embodiments, nelotanserin may not inhibit cytochrome P450. In some embodiments, there may be no clinically meaningful effect on the pharmacokinetic of probe substrate drugs of cytochrome 2B6, 2C19, and 3A4, as a potential inducer. In some embodiments, nelotanserin may be metabolized by cytochrome 3A4.

In some embodiments, a patient may have a higher baseline SAPS-PD prior to commencing treatment that is greater than 9. In additional embodiments, a patient may have a higher baseline SAPS-PD prior to commencing treatment that is greater than 15. In some embodiments, a patient may have a higher baseline SAPS-PD prior to commencing treatment that is greater than 9 and may have a nelotanserin treatment difference of −2.2 when compared to placebo. In some embodiments, a patient may have a higher baseline SAPS-PD prior to commencing treatment that is greater than 15 and may have a nelotanserin treatment difference of −5.5 when compared to placebo.

The compounds described herein may be formulated as pharmaceutical compositions that may comprise a pharmaceutically acceptable excipient. Conventional excipients, such as binding agents, fillers, acceptable wetting agents, tableting lubricants, and disintegrants may be used in tablets and capsules for oral administration. Liquid preparations for oral administration may be in the form of solutions, emulsions, aqueous or oily suspensions, and syrups. Alternatively, the oral preparations may be in the form of dry powder that can be reconstituted with water or another suitable liquid vehicle before use. Additional additives such as suspending or emulsifying agents, non-aqueous vehicles (including edible oils), preservatives, and flavorings and colorants may be added to the liquid preparations. Parenteral dosage forms may be prepared by dissolving the compound of the invention in a suitable liquid vehicle and filter sterilizing the solution before filling and sealing an appropriate vial or ampoule. These are just a few examples of the many appropriate methods well known in the art for preparing dosage forms.

The compounds described herein can be formulated into pharmaceutical compositions using techniques well known to those in the art. Suitable pharmaceutically-acceptable carriers, outside those mentioned herein, are known in the art; for example, see Remington, The Science and Practice of Pharmacy, 20th Edition, 2000, Lippincott Williams & Wilkins, (Editors: Gennaro, A. R., et al.).

While it is possible that, for use in the prophylaxis and/or treatment, a compound of the invention may, in an alternative use, be administered as a raw or pure chemical, it is preferable to present the compound or active ingredient as a pharmaceutical formulation or composition further comprising a pharmaceutically acceptable carrier.

The invention thus further provides pharmaceutical formulations comprising a compound of the invention or a pharmaceutically acceptable salt or derivative thereof together with one or more pharmaceutically acceptable carriers thereof and/or prophylactic ingredients. The carrier(s) must be “acceptable” in the sense of being compatible with the other ingredients of the formulation and not overly deleterious to the recipient thereof.

Pharmaceutical formulations include those suitable for oral, rectal, nasal, topical (including buccal and sub-lingual), vaginal or parenteral (including intramuscular, subcutaneous and intravenous) administration or in a form suitable for administration by inhalation, insulation or by a transdermal patch. Transdermal patches dispense a drug at a controlled rate by presenting the drug for absorption in an efficient manner with a minimum of degradation of the drug. Typically, transdermal patches comprise an impermeable backing layer, a single pressure sensitive adhesive and a removable protective layer with a release liner. One of ordinary skill in the art will understand and appreciate the techniques appropriate for manufacturing a desired efficacious transdermal patch based upon the needs of the artisan.

The compounds of the invention, together with a conventional adjuvant, carrier, or diluent, may thus be placed into the form of pharmaceutical formulations and unit dosages thereof, and in such form may be employed as solids, such as tablets or filled capsules, or liquids such as solutions, suspensions, emulsions, elixirs, gels or capsules filled with the same, all for oral use; in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable pharmaceutically acceptable carrier.

Compounds of the present invention or a solvate or physiologically functional derivative thereof can be used as active ingredients in pharmaceutical compositions, specifically as 5-HT_{2A} receptor modulators. The term “active ingredient” is defined in the context of a “pharmaceutical composition” and shall mean a component of a pharmaceutical composition that provides the primary pharmacological effect, as opposed to an “inactive ingredient” which would generally be recognized as providing no pharmaceutical benefit.

The dose when using the compounds of the present invention can vary within wide limits, and as is customary and is known to the physician, it is to be tailored to the individual conditions in each individual case. It depends, for example, on the nature and severity of the illness to be treated, on the condition of the patient, on the compound employed or on whether an acute or chronic disease state is treated or prophylaxis is conducted or on whether further active compounds are administered in addition to the compounds of the present invention. Representative doses of the present invention include, but are not limited to, about 0.001 mg to about 5000 mg, about 0.001 mg to about 2500 mg, about 0.001 mg to about 1000 mg, about 0.001 mg to about 500 mg, about 0.001 mg to about 250 mg, about 0.001 mg to about 100 mg, about 0.001 mg to about 50 mg, and about 0.001 mg to about 25 mg. Representative doses of the present invention include, but are not limited to, about 0.0001 to about 1,000 mg, about 10 to about 160 mg, about 10 mg, about 20 mg, about 40 mg, about 80 mg or about 160 mg. Multiple doses may be administered during the day, especially when relatively large amounts are deemed to be needed, for example 2, 3 or 4 doses. In some embodiments, the dose is administered once daily in the morning, twice daily, or once daily about 1 hour prior to the individual's bedtime. In some embodiments, the dose is administered about one to about four times per day, once daily in the morning, once daily about 1 hour prior to the individual's bedtime, or twice daily. Depending on the individual and as deemed appropriate from the patient's physician or caregiver it may be necessary to deviate upward or downward from the doses described herein.

The amount of active ingredient, or an active salt or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human. However, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, whether an acute or chronic disease state is being treated or prophylaxis is conducted, or whether further active compounds are administered in addition to the compounds of the present invention and as part of a drug combination. The dosage regimen for treating a disease condition with the compounds and/or compositions of this invention is selected in accordance with a variety of factors as cited above. Thus, the actual dosage regimen employed may vary widely and therefore may deviate from a preferred dosage regimen. One skilled in the art will recognize that dosage and dosage regimen outside these typical ranges can be tested and, where appropriate, may be used in the methods of this invention.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The daily dose can be divided, especially when relatively large amounts are administered as deemed appropriate, into several, for example 2, 3 or 4, part administrations. If appropriate, depending on individual behavior, it may be necessary to deviate upward or downward from the daily dose indicated.

The compounds of the present invention can be administrated in a wide variety of oral and parenteral dosage forms. It will be obvious to those skilled in the art that the following dosage forms may comprise, as the active component, either a compound of the invention or a pharmaceutically acceptable salt of a compound of the invention.

For preparing pharmaceutical compositions from the compounds of the present invention, the selection of a suitable pharmaceutically acceptable carrier can be either solid, liquid or a mixture of both. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted to the desired shape and size.

The powders and tablets may contain varying percentage amounts of the active compound. A representative amount in a powder or tablet may contain from 0.5 to about 90 percent of the active compound; however, an artisan would know when amounts outside of this range are necessary. Suitable carriers for powders and tablets are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier, providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized molds, allowed to cool, and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The compounds according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The pharmaceutical compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous formulations suitable for oral use can be prepared by dissolving or suspending the active component in water and adding suitable colorants, flavors, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

For topical administration to the epidermis the compounds according to the invention may be formulated as ointments, creams or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising the active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multi-dose form. In the latter case of a dropper or pipette, this may be achieved by the patient administering an appropriate, predetermined volume of the solution or suspension. In the case of a spray, this may be achieved, for example, by means of a metering atomizing spray pump.

Administration to the respiratory tract may also be achieved by means of an aerosol formulation in which the active ingredient is provided in a pressurized pack with a suitable propellant. If the compounds of the present invention or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the compounds of the present invention as an aerosol can be prepared by processes well-known to a person skilled in the art. For their preparation, for example, solutions or dispersions of the compounds of the present invention in water, water/alcohol mixtures or suitable saline solutions can be employed using customary additives, for example benzyl alcohol or other suitable preservatives, absorption enhancers for increasing the bioavailability, solubilizers, dispersants and others, and, if appropriate, customary propellants, for example include carbon dioxide, CFC's, such as, dichlorodifluoromethane, trichlorofluoromethane, or dichlorotetrafluoroethane; and the like. The aerosol may conveniently also contain a surfactant such as lecithin. The dose of drug may be controlled by provision of a metered valve.

In formulations intended for administration to the respiratory tract, including intranasal formulations, the compound will generally have a small particle size for example of the order of 10 microns or less. Such a particle size may be obtained by means known in the art, for example by micronization. When desired, formulations adapted to give sustained release of the active ingredient may be employed.

Alternatively, the active ingredients may be provided in the form of a dry powder, for example, a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler:

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are preferred compositions.

The compounds according to the invention may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethanesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like, such as those pharmaceutically acceptable salts listed in Journal of Pharmaceutical Science, 66, 2 (1977); incorporated herein by reference in its entirety.

The acid addition salts may be obtained as the direct products of compound synthesis. In the alternative, the free base may be dissolved in a suitable solvent containing the appropriate acid, and the salt isolated by evaporating the solvent or otherwise separating the salt and solvent. The compounds of this invention may form solvates with standard low molecular weight solvents using methods known to the skilled artisan.

Compounds of the present invention can be converted to “pro-drugs.” The term “pro-drugs” refers to compounds that have been modified with specific chemical groups known in the art, and when administered into an individual, these groups undergo biotransformation to give the parent compound. Pro-drugs can thus be viewed as compounds of the invention containing one or more specialized non-toxic protective groups used in a transient manner to alter or to eliminate a property of the compound. In one general aspect, the “pro-drug” approach is utilized to facilitate oral absorption. A thorough discussion is provided in T. Higuchi and V. Stella, “Pro-drugs as Novel Delivery Systems,” Vol. 14 of the A. C. S. Symposium Series; and in Bioreversible Carriers in Drug Design, ed. Edward B. Roche, American Pharmaceutical Association and Pergamon Press, 1987, both of which are hereby incorporated by reference in their entirety.

Some embodiments of the present invention include a method of producing a pharmaceutical composition for “combination-therapy” comprising admixing at least one compound according to any of the compound embodiments disclosed herein, together with at least one known pharmaceutical agent as described herein and a pharmaceutically acceptable carrier.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the individual matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using “consisting of” or “consisting essentially of” language, rather than “comprising”. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The invention will be described in greater detail by way of specific examples. The following examples are offered for illustrative purposes, and are not intended to limit the invention in any manner.

### Example 1

As depicted in FIG. 1-5, Nelotanserin inhibited serotonin-induced 5HT_{2A}R-β-arrestin 2 interactions, acting as a potent antagonist of the recruitment of β-arrestin 2 to the 5HT2A receptor (IC50 (SEM): 3.74 nM (±0.65)), and was found to be more potent than clozapine (IC50 (SEM): 67.8 nM (±12.1)). In mice, nelotanserin blocked DOI-induced head twitches in a dose-dependent manner (ED50 (95% CL): 2.4 mg/kg (1.3-4.4)), demonstrating potent in vivo antagonism of the 5HT2A agonist-induced behavior. Finally, in a mouse model of psychosis-like behavior, PCP-induced hyperlocomotor activity was effectively inhibited by nelotanserin (ED50 (95% CL): 3.0 mg/kg (1.7-5.5)), without any effect of nelotanserin on locomotor behavior on its own. This effect was comparable to the results obtained with clozapine. No significant effects on head twitches or locomotor activity were observed upon treatment of nelotanserin followed by vehicle. The potency for antagonizing 5-HT induced βarrestin2 recruitment is further illustrated in Table 1.

TABLE 1

| Compound | | SEM | N |
|---|---|---|---|
| | EC50 (nM) | | |
| 5HT | 49.47 | 11.65 | 9 |
| clozapine | | | |
| nelotanserin | | | |
| pimavanserin | | | |
| clozapine + 1 uM 5HT | 67.82 | 12.21 | 6 |
| Nelotanserin + 1 uM 5HT | 3.74 | 0.65 | 7 |
| Pimavanserin + 1 uM 5HT | 12.12 | 3.25 | 3 |
| | % Max of 5HT | | |
| 5HT | 98.20 | 1.27 | 9 |
| clozapine | −2.27 | 1.04 | 6 |
| nelotanserin | −2.21 | 0.96 | 7 |
| pimavanserin | −2.56 | 1.24 | 2 |
| clozapine + 1 uM 5HT | 95.98 | 5.18 | 6 |
| Nelotanserin + 1 uM 5HT | 98.53 | 7.02 | 7 |
| Pimavanserin + 1 uM 5HT | 83.52 | 6.00 | 3 |
| | Fold | | |
| 5HT | 7.43 | 0.82 | 9 |
| clozapine | 0.89 | 0.08 | 6 |
| nelotanserin | 0.90 | 0.06 | 7 |
| pimavanserin | 0.85 | 0.10 | 2 |
| clozapine + 1 uM 5HT | 8.08 | 0.64 | 7 |
| Nelotanserin + 1 uM 5HT | 7.63 | 0.85 | 7 |
| Pimavanserin + 1 uM 5HT | 5.60 | 0.63 | 3 |

FIG. 1 illustrates a 5-HT2A β-arrestin 2 to the 5HT2A receptor enzyme fragment complementation (EFC) assay in h5HTA-Dx-U2OS cells (n=3-9). The h5HTA-Dx-U2OS cells were plated 5,000 cells/well in a 384-well plate the day prior to drug treatment. 5-HT was prepared fresh for each experiment, diluted in 20 mM ascorbic acid, then prepared at 5× concentrations in assay buffer. Drug treatment was performed for 90 minutes at 37 degrees celcius followed by addition of DiscoverX detection reagent for 60 minutes at room temperature, after which luminescence was measured.

### Example 2

FIGS. 2-5 illustrate Mouse data—Male C57BL6J mice from JAX˜12 weeks old. Studies were randomized and investigator was blinded. The figures contain three ways of analyzing the data obtained from the Versamax open field activity monitors on the same cohort of mice. The infrared beams in the chambers measure breaks in X, Y, and Z planes. Typically data are reported for Horizontal beam breaks and sometimes for distance travelled and stereotypy in studies involving PCP.

FIG. 2 illustrates total distance of PCP-stimulated locomotor activity. Male C57BL6 mice from JAX were used at 12-15 weeks of age, n=6 for all conditions). All mice were naïve at the time of testing. Mice were hadituabted to activity chambers for 30 minutes, then treated with clozapine (CLZ, 1 mg/kg, i.p.), nelotanserin (NTS), 1-10 mg/kg, i.p.) or vehicle (V, 1:1:8 DMSO:Tween-80:diH2O, i.p.). Activity was recorded again for 30 minutes (30 minute pretreatment with drug after 30 minute hadituation). Mice were then treated with 0.9% saline (i.p.) or PCP (10 mg/kg, i.p.) and monitored for an additional 60 minutes. All measurements were recorded by VersaMax software, and experimenter was blinded to treatment. The summary figure is a compilation of distance travelled over the last 60 minutes with drug treatment. A measure of nelotanserin or clozapine alone reveals no effects of the drugs in the first 30 minutes pretreatment (not shown here, but data are collected). Data analyzed via two-way ANOVA followed by Sidak post hoc test to assess statistical differences between animals which received saline of PCP following nelotanserin, clozapine, or vehicle. ED 50 (95% confidence limits)=3.0 (1.7-5.5) with regards to total distance traveled data of FIG. 2.

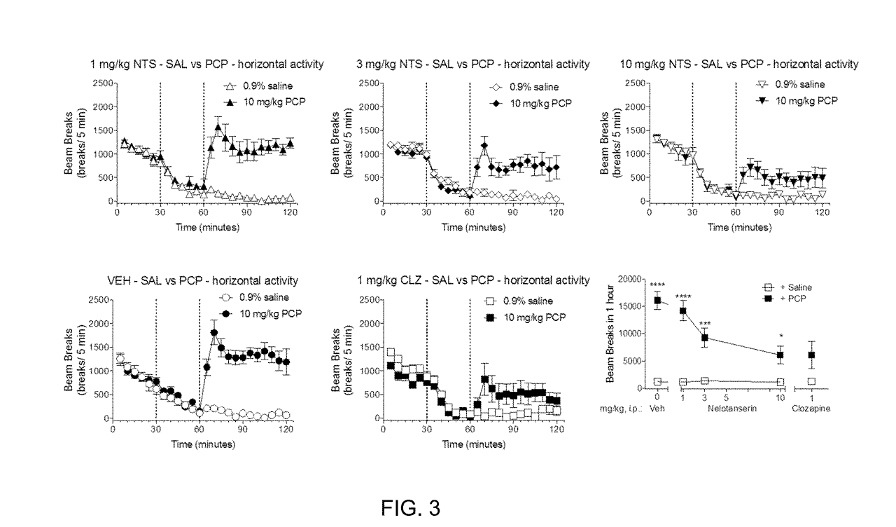

FIG. 3 illustrates horizontal beam breaks of PCP-stimulated locomotor activity. Male C57BL6 mice from JAX were used at 12-15 weeks of age, n=6 for all conditions). All mice were naïve at the time of testing. Mice were hadituabted to activity chambers for 30 minutes, then treated with clozapine (CLZ, 1 mg/kg, i.p.), nelotanserin (NTS), 1-10 mg/kg, i.p.) or vehicle (V, 1:1:8 DMSO:Tween-80:diH2O, i.p.). Activity was recorded again for 30 minutes (30 minute pretreatment with drug after 30 minute hadituation). Mice were then treated with 0.9% saline (i.p.) or PCP (10 mg/kg, i.p.) and monitored for an additional 60 minutes. All measurements were recorded by VersaMax software, and experimenter was blinded to treatment. The summary figure is a compilation of the sum of beam breaks (X, Y plane) monitored over the last 60 minutes. A measure of nelotanserin or clozapine alone reveals no effects of the drugs in the first 30 minutes pretreatment (not shown here, but data are collected). Data analyzed via two-way ANOVA followed by Sidak post hoc test to assess statistical differences between animals which received saline of PCP following nelotanserin, clozapine, or vehicle. ED 50 (95% confidence limits)=4.3 (2.7-7.9) with regards to beam breaks data of FIG. 3.

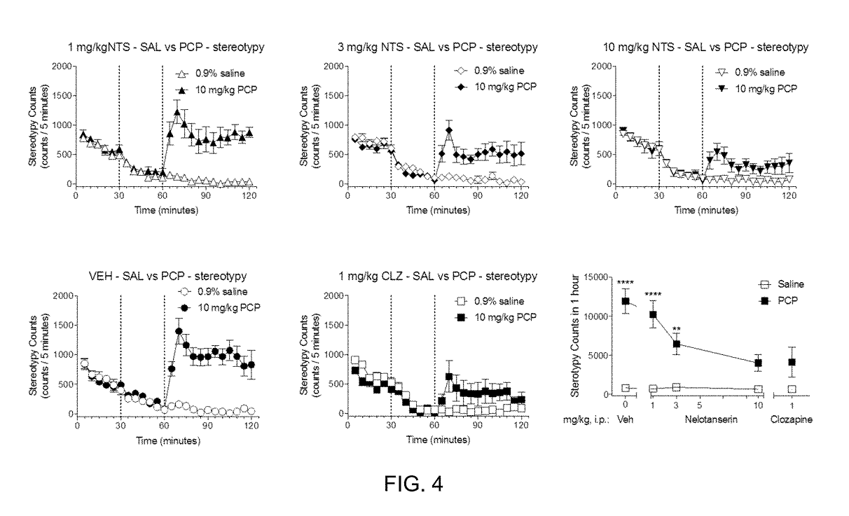

FIG. 4 illustrates stereotypy counts (beam breaks in X, Y, Z direction) of PCP-stimulated locomotor activity. Male C57BL6 mice from JAX were used at 12-15 weeks of age, n=6 for all conditions). All mice were naïve at the time of testing. Mice were hadituabted to activity chambers for 30 minutes, then treated with clozapine (CLZ, 1 mg/kg, i.p.), nelotanserin (NTS), 1-10 mg/kg, i.p.) or vehicle (V, 1:1:8 DMSO:Tween-80:diH2O, i.p.). Activity was recorded again for 30 minutes (30 minute pretreatment with drug after 30 minute hadituation). Mice were then treated with 0.9% saline (i.p.) or PCP (10 mg/kg, i.p.) and monitored for an additional 60 minutes. All measurements were recorded by VersaMax software, and experimenter was blinded to treatment. The summary figure is a compilation of the sum of beam breaks (X, Y, Z stereotypy measures) monitored over the last 60 minutes. A measure of nelotanserin or clozapine alone reveals no effects of the drugs in the first 30 minutes pretreatment (not shown here, but data are collected). Data analyzed via two-way ANOVA followed by Sidak post hoc test to assess statistical differences between animals which received saline of PCP following nelotanserin, clozapine, or vehicle. ED 50 (95% confidence limits)=3.8 (2.0-7.1) with regards to stereotypy counts of FIG. 4.

### Example 3

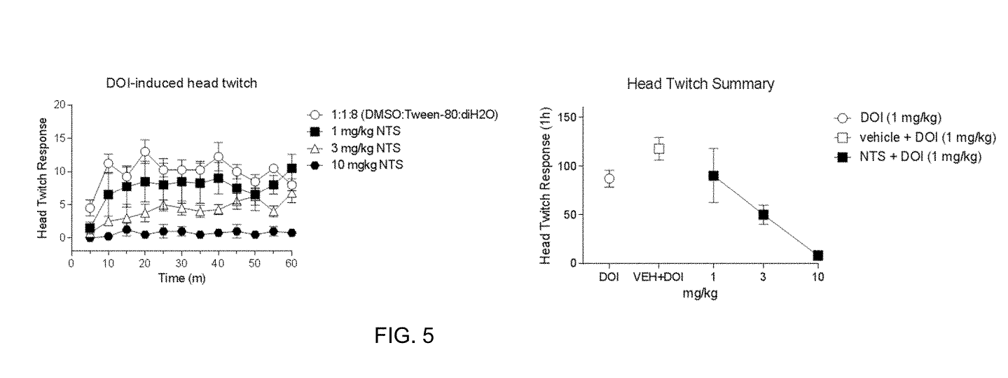

FIG. 5 illustrates DOI-induced head twitch response. Male C57BL6 mice from JAX were used at 12-15 weeks of age, n=4 for all conditions). All mice were naïve at the time of testing. Mice were injected with nelotanserin (1-10 mg/kg, i.p.) or vehicle (1:1:8 DMSO:Tween-80:diH2O) 30 minutes prior to injection with DOI (1 mg/kg, i.p., saline 0.9%). After DOI injection, mice were placed in clear plexiglass cylinders (6″ diameter, 16″ height) on filter paper and observed for 1 hour for head twitch responses. Data are expressed as mean+SEM in 5 minute bins. Mice were observed during 30 minute pre-treatment. No head twitches were recorded in any mouse. Observers were blinded to experimental conditions. ED 50 (95% confidence limits)=2.4 (1.3-4.4) with regards to DOI-induced head twitch of FIG. 5. Table 2 below illustrates the number of mice used and number needed for the DOI-induced head twitch.

TABLE 2

| Pre-Tx | Tx | Current N | To be added |
|---|---|---|---|
| None | 1 mg/kg DOI | 4 | 0 |
| Vehicle | 1 mg/kg DOI | 4 | 2 |
| 1 mg/kg NTS | 1 mg/kg DOI | 4 | 2 |
| 3 mg/kg NTS | 1 mg/kg DOI | 4 | 2 |
| 10 mg/kg NTS | 1 mg/kg DOI | 4 | 2 |
| 1 mg/kg CLZ | 1 mg/kg DOI | 0 | 6 |
| | | total | 14 |

### Example 4

Inverse agonists/antagonists at the serotonin 2A receptor (5HT2AR) have proven to be highly effective, mainstay therapies for the treatment of psychosis for decades. Many atypical antipsychotics, including clozapine, have been explored for their efficacy in treating a wide range of neuropsychiatric deficits. However, atypical antipsychotics target multiple receptors, and in the case of clozapine, demonstrate activity at dopamine and 5HT2C receptors. Nelotanserin is a 5HT2A inverse agonist with 80-fold selectivity over 5HT2C that was shown to be inactive in a panel of 66 receptors and ion channels. Nelotanserin is being investigated in a Phase 2 clinical trial of subjects diagnosed with Lewy body dementia who experience frequent visual hallucinations. In the nonclinical study presented here, we evaluated the in vitro characteristics of nelotanserin and examined its effects in mouse models of 5HT2AR activation and psychosis.

The competitive properties at the 5HT_{2A}R of nelotanserin were characterized with the DiscoveRx PathHunter β-arrestin2 enzyme fragment complementation assay in h5HT_{2A}-Dx-U2OS cells (n=6-7). Cells were incubated with nelotanserin or clozapine for 90 minutes at 37° C. followed by addition of DiscoveRx detection reagent for 60 minutes, after which luminescence was measured. In vivo effects were assessed in male C57Bl/6J mice by 1) inhibition of DOI (1-(2,5-dimethoxy 4-iodophenyl)-2-amino propane hydrochloride)-induced head twitch response and 2) inhibition of PCP-induced hyperlocomotion. In the DOIinduced head twitch assay, mice were administered nelotanserin or vehicle 30 minutes prior to administration of the 5HT2A receptor agonist, DOI (1 mg/kg, i.p.) and observed for head twitch response over 1 hour. PCP-induced hyperlocomotion was measured in open field activity monitor chambers. Mice were administered nelotanserin, clozapine, or vehicle (i.p.), and their activity was monitored for 30 minutes before administration of either PCP (10 mg/kg, i.p.) or saline (i.p.) followed by an additional 30 minutes of activity monitoring. All studies were performed in a blinded and randomized manner and potencies were calculated. Clozapine was used as the basis for comparison. Results: Nelotanserin inhibited serotonin-induced 5HT2AR-β-arrestin 2 interactions, acting as a potent antagonist of the recruitment of β-arrestin 2 to the 5HT2A receptor (IC50 (SEM): 3.74 nM (±0.65)), and was found to be more potent than clozapine (IC50 (SEM): 67.8 nM (±12.1)). In mice, nelotanserin blocked DOI-induced head twitches in a dose-dependent manner (ED50 (95% CL): 2.4 mg/kg (1.3-4.4)), demonstrating potent in vivo antagonism of the 5HT2A agonist-induced behavior. Finally, in a mouse model of psychosis-like behavior, PCP-induced hyperlocomotion was effectively inhibited by nelotanserin (ED50 (95% CL): 3.0 mg/kg (1.7-5.5)). No effect of nelotanserin alone was observed on locomotor behavior following administration of saline. This effect was comparable to the results obtained with clozapine. No significant effects on head twitches or locomotor activity were observed upon treatment of nelotanserin followed by vehicle.

Conclusions: Nelotanserin antagonizes β-arrestin 2 recruitment in cell-based 5HT2A signaling assays and in mouse models blocks the effects of 5HT2A agonism (DOI-induced head twitch) and of psychosis-like behavior (PCP-induced hyperlocomotion). In all three assays, nelotanserin acts in a manner similar to clozapine, a clinically relevant atypical antipsychotic. These data suggest that nelotanserin may prove useful in the treatment of psychotic symptoms.

### Example 5

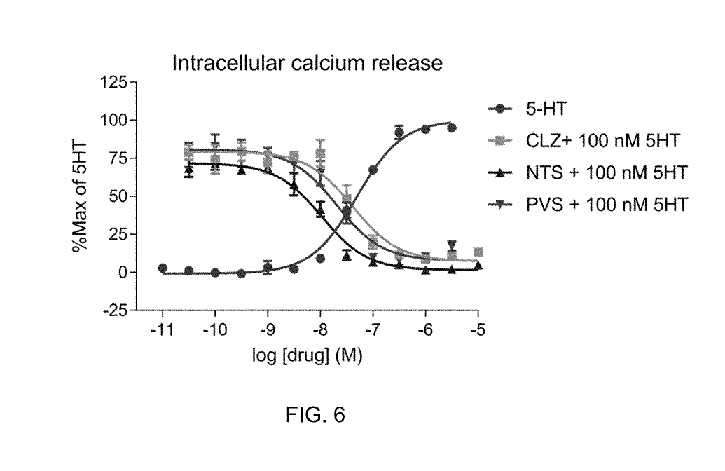

As illustrated in FIG. 6, Nelotanserin inhibited serotonin-induced intracellular calcium release. FIG. 6 further illustrates nelotanserin (NTS) is about 2-fold more potent than pimavanserin (PVS) or clozapine (CLZ).

What is claimed:

1. A method for the prophylaxis and/or treatment of the psychosis or psychotic symptoms comprising administering to said individual in need thereof a therapeutically effective amount of a 5-HT_{2A} serotonin receptor inverse agonist.

2. The method of claim 1, wherein the psychotic symptoms are associated with dementia with Lewy bodies, Parkinson's disease, or Parkinson's disease dementia.

3. The method of claim 1, wherein the psychotic symptoms are hallucinations, delusions, or a combination thereof.

4. The method of claim 3, wherein the hallucinations are selected from visual hallucinations, auditory hallucinations, olfactory hallucinations, gustatory hallucinations, tactile hallucinations, proprioceptive hallucinations, equilibrioceptive hallucinations, nociceptive hallucinations, thermoceptive hallucinations, chronoceptive hallucinations and any combination thereof.

5. The method of claim 1, wherein the 5-HT_{2A} serotonin receptor inverse agonist is nelotanserin, or a pharmaceutically acceptable salt, hydrate, polymorph, or solvate thereof.

6. The method of claim 5, wherein the therapeutically effective amount of nelotanserin or a pharmaceutically acceptable salt, hydrate, polymorph, or solvate thereof is from about 10 mg to about 160 mg.

7. The method of claim 5, wherein the therapeutically effective amount of nelotanserin or a pharmaceutically acceptable salt, hydrate, polymorph, or solvate thereof is about 10 mg, about 20 mg, about 40 mg, about 80 mg, or about 160 mg.

8. The method of claim 5, wherein the therapeutically effective amount of nelotanserin or a pharmaceutically acceptable salt, hydrate, polymorph, or solvate thereof is about 10 mg.

9. The method of claim 5, wherein the therapeutically effective amount of nelotanserin or a pharmaceutically acceptable salt, hydrate, polymorph, or solvate thereof is about 20 mg.

10. The method of claim 5, wherein the therapeutically effective amount of nelotanserin or a pharmaceutically acceptable salt, hydrate, polymorph, or solvate thereof is about 40 mg.

11. The method of claim 5, wherein the therapeutically effective amount of nelotanserin or a pharmaceutically acceptable salt, hydrate, polymorph, or solvate thereof is about 80 mg.

12. The method of claim 5, wherein the therapeutically effective amount of nelotanserin or a pharmaceutically acceptable salt, hydrate, polymorph, or solvate thereof is about 160 mg.

13. The method of claim 1, wherein the therapeutically effective amount of a 5-HT_{2A} serotonin receptor inverse agonist is administered once a day, twice a day, three times a day, or four times a day.

14. The method of claim 1, wherein the therapeutically effective amount of a 5-HT_{2A} serotonin receptor inverse agonist is configured for immediate release, for extended release, for delayed release, or any combination thereof.

15. The method of claim 1, wherein the therapeutically effective amount of a 5-HT_{2A} serotonin receptor inverse agonist is formulated for oral administration.

16. The method of claim 1, wherein the individual is a human.

17. The method of claim 1, wherein the individual has an SAPS-PD baseline score greater than 9, greater than 15, or a combination thereof.

18. The method of claim 1, wherein the individual has an SAPS-PD baseline score greater than 9 and a nelotanserin treatment difference of −2.2 when compared to placebo.

19. The method of claim 1, wherein the individual has an SAPS-PD baseline score greater than 15 and a nelotanserin treatment difference of −2.2 when compared to placebo.

20. A method for the prophylaxis and/or treatment of the Parkinson's disease psychosis and the symptoms thereof, comprising administering to said individual in need thereof a therapeutically effective amount of a 5-HT_{2A} serotonin receptor inverse agonist.

21. The method of claim 20, wherein the symptoms are hallucinations, delusions, or a combination thereof.

22. The method of claim 20, wherein the hallucinations are selected from visual hallucinations, auditory hallucinations, olfactory hallucinations, gustatory hallucinations, tactile hallucinations, proprioceptive hallucinations, equilibrioceptive hallucinations, nociceptive hallucinations, thermoceptive hallucinations, chronoceptive hallucinations and any combination thereof.

23. The method of claim 20, wherein the 5-HT_{2A} serotonin receptor inverse agonist is nelotanserin, or a pharmaceutically acceptable salt, hydrate, polymorph, or solvate thereof.

24. The method of claim 23, wherein the therapeutically effective amount of nelotanserin or a pharmaceutically acceptable salt, hydrate, polymorph, or solvate thereof is from about 10 mg to about 160 mg.

25. The method of claim 23, wherein the therapeutically effective amount of nelotanserin or a pharmaceutically acceptable salt, hydrate, polymorph, or solvate thereof is about 10 mg, about 20 mg, about 40 mg, about 80 mg, or about 160 mg.

26. The method of claim 23, wherein the therapeutically effective amount of nelotanserin or a pharmaceutically acceptable salt, hydrate, polymorph, or solvate thereof is about 10 mg.

27. The method of claim 23, wherein the therapeutically effective amount of nelotanserin or a pharmaceutically acceptable salt, hydrate, polymorph, or solvate thereof is about 20 mg.

28. The method of claim 23, wherein the therapeutically effective amount of nelotanserin or a pharmaceutically acceptable salt, hydrate, polymorph, or solvate thereof is about 40 mg.

29. The method of claim 23, wherein the therapeutically effective amount of nelotanserin or a pharmaceutically acceptable salt, hydrate, polymorph, or solvate thereof is about 80 mg.

30. The method of claim 23, wherein the therapeutically effective amount of nelotanserin or a pharmaceutically acceptable salt, hydrate, polymorph, or solvate thereof is about 160 mg.

31. The method of claim 20, wherein the therapeutically effective amount of a 5-HT_{2A} serotonin receptor inverse agonist is administered once a day, twice a day, three times a day, or four times a day.

32. The method of claim 20, wherein the therapeutically effective amount of a 5-HT_{2A} serotonin receptor inverse agonist is configured for immediate release, for extended release, for delayed release, or any combination thereof.

33. The method of claim 20, wherein the therapeutically effective amount of a 5-HT_{2A} serotonin receptor inverse agonist is formulated for oral administration.

34. The method of claim 18, wherein the individual is a human.

35. The method of claim 20, wherein the individual has an SAPS-PD baseline score greater than 9, greater than 15, or a combination thereof.

36. The method of claim 20, wherein the individual has an SAPS-PD baseline score greater than 9 and a nelotanserin treatment difference of −2.2 when compared to placebo.

37. The method of claim 20, wherein the individual has an SAPS-PD baseline score greater than 15 and a nelotanserin treatment difference of −2.2 when compared to placebo.
